(19) United States
(12) Patent Application Publication
(10) Pub. No.: US 2009/0297441 A1
(43) Pub. Date: Dec. 3, 2009

(54) Imaging Agents

(76) Inventors: Leigh Trevor Canham, Malvern (GB); Anna Agnieszka Kluczewska, Subiaco (AU); Jerome Paul Barley, Subiaco (AU); Raphaela Fortes Drummond Chicarino Varajao, Subiaco (AU)

Correspondence Address: NIXON & VANDERHYE, PC, 901 NORTH GLEBE ROAD, 11TH FLOOR, ARLINGTON, VA, 22203, US

(21) Appl. No.: 11/992,292

(22) PCT Filed: Sep. 22, 2006
(86) PCT No.: PCT/GB2006/003522

§ 371 Date: Jul. 28, 2009

(30) Foreign Application Priority Data
Sep. 22, 2005 (GB) 0519391.7

(51) Int. Cl. A61K 51/00; A61K 49/00; A61K 49/04

(52) U.S. Cl. 424/1.61; 424/9.1; 424/9.3; 424/9.4; 424/9.5; 424/9.6

(57) ABSTRACT

The use of silicon as an imaging agent is described.

### FIELD OF THE INVENTION

The present invention relates to the use of silicon, in particular porous silicon (pSi), as an imaging agent for use in combination with one or more of a range of imaging techniques or modalities. In particular, the silicon imaging agent may be used as a contrast agent suitable for use in human or animal circulatory and other organ systems including the vasculature, respiratory, alimentary, lymphatic, musculoskeletal, reproductive, nervous and renal/urinary systems, and for marking skin and other tissues. The silicon imaging agent is also suitable for use in molecular imaging.

### BACKGROUND OF THE INVENTION

Imaging agents are materials used to improve the visibility, in particular, the contrast, of internal bodily structures in an image generated using any one of a range of imaging techniques. These techniques or modalities include, but are not limited to, x-rays, computerised tomography (CT), magnetic resonance imaging (MRI), scintigraphy, fluorescence and ultrasound.

Imaging agents may also be used to accurately position a body, or part of a body, including an organ or tissue, in the correct orientation or field of view for imaging or therapeutic treatment.

Specific biophysical properties of imaging agents are exploited to render them visible and provide contrast to other anatomical structures under a particular modality. The sorts of properties of the agents that are utilised include, for example, the density and weight of the agent for use with x-rays, the ultrasonic echo that the agent generates for use in ultrasound and the magnetic properties of the agent when compared with normal tissue, for use in magnetic resonance imaging (MRI).

One well known type of imaging agent is based on barium sulphate, which may be mixed with a range of ingredients to provide an opaque white mixture. The barium sulphate is said to be radiopaque because it prevents, or attenuates, the passage of electromagnetic radiation such as x-rays or similar radiation to pass through. Barium sulphate is used in the digestive tract and is therefore usually swallowed or administered as an enema.

Other known types of imaging agents are based on iodine. Contrast agents such as iohexyl, iodixanaol plus many others are generally available as clear colourless aqueous solutions. Many modern iodinated contrast agents can be used almost anywhere in the body. Most often they are used intravenously, but for various purposes they can also be used intra-arterially, intra-thecally and intra-abdominally, i.e. intra-peritoneally.

The imaging agents described may be administered via a needle (including a microneedle), catheter, nebuliser and may be in particulate, aerosol, liquid or solid forms. They may also be placed during open surgical or laparoscopic procedures.

There is a broad range of imaging techniques currently available. Examples of known medical imaging techniques and systems include x-ray, computed tomographic (CT) x-ray imaging, ultrasound, transcranial colour coded sonography, portal film imaging devices, electronic portal imaging devices, electrical impedance tomography (EIT), brain electrical activity mapping (BEAM), magnetoelectro-encephalography (MEG), nuclear medicine (NM) including positron emission tomography (PET) and single photon emission computed tomography (SPECT), magnetic source imaging (MSI), magnetic resonance spectroscopy (MRS), thermal imaging, infrared imaging, optical imaging, fluorescence imaging, laser optical imaging, magnetic resonance imaging (MRI), magnetic resonance mammography (MR mammography), electric potential tomography (EPT), magnetic resonance angiography (MRA), arterial contrast injection angiography and digital subtraction angiography. It is also possible to combine some of these techniques, for example, PET and MRI, PET and CT, SPECT and CT. These techniques have provided the medical profession with improved visualization of the anatomical structure of portions of the human and animal body without necessarily having to perform invasive surgical techniques. Certain techniques, such as MRS, MEG and PET give functional rather than anatomical tissue information to clinicians. Some of the more advanced techniques are also being integrated with more traditional imaging modalities, such as x-ray (e.g. mammography and fluoroscopy), ultrasound and video imaging.

The use of the above-mentioned imaging modalities for obtaining images, marking and analyzing anatomical structures and assessing tissue function is becoming increasingly prominent in many medical procedures. For example, in the field of neurosurgery, prior to performing surgery, a three-dimensional image of a patient's head and brain may be formed using a CT imaging system. The CT image may be used by the surgeon in establishing a three-dimensional frame of reference for the operation and for planning any surgery. Functional data from MRS and PET can be further combined to the three dimensional anatomical map to produce an anatometabolic map. These imaging modalities are also used in the field of oncology for the identification, planning, staging, treatment and monitoring of lesions or other areas of abnormal tissue. For example, imaging modalities currently used in the diagnosis and monitoring of breast lesions include mammography, ultrasound, and, more recently, MRI and/or MR mammography. These imaging modalities may be used in assessing the treatment of lesions by, for example, chemotherapy, surgery and radiation therapy. For example, when a patient is treated with chemotherapy, drugs are introduced into the patient's body to destroy the lesion. During the course of this treatment, a variety of imaging modalities may be implemented to follow the progress of the treatment or condition by comparing a series of images of a particular treatment site over time. Positional information obtained from the images may be used before and/or during the performance of a medical procedure at the site of the lesion. After a lesion is removed by surgical methods, one or more imaging modalities may be useful in imaging the site of lesion removal, and/or the whole body, to monitor the condition of the site and the patient. Imaging of the removed sample can also be used to ensure the lesion of interest has been successfully removed and is contained within the surgical specimen.

These imaging modalities may be used in radiation therapy. Radiation therapy involves subjecting a lesion to x-ray, proton or electron radiation through the use of, for example, a linear accelerator. In radiation therapy, geometric accuracy is an important factor in achieving a successful outcome following treatment. The objective of radiation therapy is to target a specific area, such as a neoplastic or cancerous lesion, whilst minimising radiation to healthy tissue. An important factor in precisely targeting a lesion site and avoiding healthy tissue is proper positioning of a patient in reference to the radiation-producing apparatus. The use of one or more imaging modalities has become an important component in properly positioning a patient for radiation therapy because such imaging may provide multiple data sets for positioning the patient and may provide for improved patient positioning over multiple treatments.

An increasingly important factor in utilizing these various imaging modalities for non-invasive medical procedures is the ability to create, interpret, compare, fuse, and/or to integrate images to obtain positional information about a portion of the body or an anatomical site of a patient. Such “body mapping” or “multi-modality image fusion” techniques use various data points or positional locators on or in the body in order to pinpoint the exact location in which a particular technique is to be performed. For example, body positioning techniques for radiation therapy often involve taking a reference image of a patient's body prior to radiation therapy, and then visually comparing or electronically integrating or synthesizing the reference image with subsequent images of a patient's body position in order to properly position the patient each time radiation therapy is performed.

It is possible for an imaging agent, such as a tissue marker, to provide either positive or negative contrast in relation to surrounding tissues. Positive contrast refers to the situation where the marker is detectable as a lighter shade of contrast on a darker background shade of tissue, whereas negative contrast refers to the situation where a marker is detectable as a darker shade of contrast on a lighter background shade of tissue. For example, and as mentioned previously, one well known type of imaging agent is based on barium sulphate, which may be mixed with a range of ingredients to provide an opaque white mixture. The barium sulphate is radiopaque, which means that it prevents the passage of electromagnetic radiation such as x-rays or similar radiation to pass through and therefore it provides a positive contrast. When used in examination of the large bowel, barium sulphate is often used in combination with air. The air in the bowel provides a negative contrast to the barium sulphate which coats the bowel wall, thereby improving visualisation of the bowel mucosal surface. Both positive and negative contrast can be equally useful in connection with the use of imaging agents, such as tissue markers.

In MRI, contrast agents are chemical substances introduced to the anatomical or functional region being imaged, in order to increase the differences between different tissues or between normal and abnormal tissue, through higher contrast in the image produced, by altering the relaxation times. MRI contrast agents are classified by the different changes in relaxation times after their injection.

Positive contrast agents cause a reduction in the T1 relaxation time (i.e. increased signal intensity on T1 weighted images). Positive contrast agents appear bright on MRI and are typically small molecular weight compounds often containing as their active element Gadolinium, Manganese, or Iron. All of these elements have unpaired electron spins in their outer shells and long relaxivities. Contrast agents such as gadopentetate dimeglumine, gadoteridol, and gadoterate meglumine, are utilized for the central nervous system and the complete body. Mangafodipir trisodium is specially used for lesions of the liver and gadodiamide is used for the central nervous system.

Negative contrast agents, that appear predominantly dark on MRI, are small particulate aggregates often termed superparamagnetic iron oxide (SPIO). These agents produce, predominantly, spin relaxation effects (local field inhomogeneities), which result in shorter T1 and T2 relaxation times. SPIOs and ultrasmall superparamagnetic iron oxides (USPIO) usually consist of a crystalline iron oxide core containing thousands of iron atoms and a shell of polymer, e.g. dextran, polyethyleneglycol, and produce very high T2 relaxivities. USPIOs smaller than 300 nm cause a substantial T1 relaxation.

Negative contrast may also be referred to as a signal void in MRI. The MRI appearance of solid dry objects is as dense negative contrast or black signal voids within the tissue. This is because MRI requires the presence of molecular mobile hydrogen linked molecules such as water to produce positive contrast. Solid dry objects will not return signal and will therefore appear as a signal void or negative contrast.

Another group of negative contrast agents i.e. they appear dark on MRI, are perfluorochemicals such as perfluorocarbons. Their presence excludes the hydrogen atoms responsible for the signal in MRI.

One difficulty in the use of imaging agents for procedures utilizing multiple imaging modalities is that an imaging agent, such as a tissue marker, that is detectable in and compatible with one imaging modality (e.g. x-ray) may not be detectable in or compatible with another imaging modality (e.g. MRI). Alternatively, the marker may be detectable, but may cause substantial distortion or interference with images formed by certain imaging modalities. Furthermore, certain markers may pose a safety risk to a patient exposed to certain imaging modalities such as MRI.

For example, conventional markers such as titanium markers or stainless steel, may be detectable in and compatible with x-ray and other non-magnetic field imaging modalities, but may not be compatible with images produced via magnetic field imaging modalities such as MRI. More specifically, the interaction of the magnetic and/or conductive properties of the marker with the magnetic field applied during MRI may cause image distortion. Image distortion and heating of the marker are notable with markers containing ferromagnetic materials, paramagnetic materials or other materials of high magnetic susceptibility. These materials may also pose a safety risk associated with the exposure of the marker to external or applied magnetic fields, such as movement of the marker within the body.

Another difficulty in the use of imaging agents for procedures utilizing multiple imaging modalities is that an imaging agent, such as a tissue marker, that is detectable in and compatible with more than one imaging modality (e.g. x-ray and ultrasound) may not be detectable in, or compatible with, yet other imaging modalities (e.g. MRI). Furthermore, if an imaging agent is visible under more than one modality, said marker, by merit of its component substances, may not be fully biodegradable or bioresorbable. Alternatively, the marker may be detectable with more than one modality, but may cause substantial distortion or interference with images formed by other imaging modalities.

For example, some currently available markers may use a combination of a metal and a biodegradable polymer to ensure visibility and compatibility with x-ray and other imaging modalities, but may not be completely biodegradable or may not be compatible with images produced via magnetic field imaging modalities such as MRI.

It would be advantageous to provide an imaging agent which may be fully biodegradable and yet still detectable in and compatible with a range of imaging modalities and, in certain circumstances, with magnetic and non-magnetic field imaging modalities such that images from one or more imaging modalities may be obtained for use in a variety of medical procedures.

It would also be advantageous to provide an imaging agent which is visible under most imaging modalities and which is comprised of only one or substantially one component.

As mentioned above, a tissue marker may be viewed as a type of imaging agent. Generally, tissue marking is a method of marking a position in a body, such as a specific position in a tissue or organ, in order to allow re-visiting of the position to check for progress or developments of an ailment or a treatment, or to allow re-treatment at the same site. For example, tissue marking can be used during a biopsy or other tissue-removal procedure to accurately mark the site of the tissue-removal or biopsy, thus allowing later return to the same site if desired in order, for example, to monitor the status of the tissue in question, or to carry out a further biopsy. A tissue marker may be viewed, broadly, as a type of imaging agent that does not move or stays substantially in the same position once it has been administered or implanted. It is often desirable that the tissue marker is biodegradable over a period of time and is resorbed safely by the body.

In diagnosing and treating certain medical conditions, it is often desirable to perform a biopsy, in which a specimen or sample of tissue is removed for analysis. Obtaining a tissue sample by biopsy and the subsequent examination are typically employed in the diagnosis of cancers and other malignant tumours, or to confirm that a suspected lesion or tumour is not malignant. The information obtained from these diagnostic tests and/or examinations is frequently used to devise a therapeutic plan for the appropriate surgical procedure or other course of treatment.

In many instances, the suspicious tissue to be sampled is located in a subcutaneous site, such as inside a human breast. Such removal of tissue samples may be accomplished by open surgical technique, or through the use of a specialized biopsy instrument and techniques. To minimize surgical intrusion into the patient's body, it is often desirable to insert a small instrument, such as a biopsy needle, into the body for extracting the biopsy specimen while imaging the procedure using fluoroscopy, ultrasonic imaging, x-rays, MRI or any other suitable form of imaging technique. Tissue marking may be useful in a range of procedures including biopsy procedures. In particular, tissue marking may be useful in biopsy procedures involving the colon, rectum, prostate, breast, brain, kidneys, liver, lungs, bone, oropharynx, skin, lymph nodes, spleen, adrenals, testis, ovaries, ureter, nerve, bladder, heart, spleen and soft tissues in general, including muscles. Examination of tissue samples taken by biopsy is of significance in the diagnosis and treatment of all cancers, most commonly breast cancer, colorectal cancer, prostate cancer, ovarian cancer, skin cancer (including melanoma) and lung cancer.

It is, at times, seen in modern breast biopsies that evidence of a lesion is removed during biopsy. Removing all trace of the tissue also removes identifying features from the site, and makes it difficult to return to the same location later, to re-check the site. This problem, created by a removal of a potentially malignant breast mass or cluster of microcalcifications during core biopsy, can be addressed by placing tissue markers immediately after or during the biopsy procedure. The marker, e.g., a radiopaque material, can be used to help locate the biopsy site in case malignancy is determined, thereby enabling return to the same site and optionally a subsequent treatment such as surgical excision, even if the mammographic findings associated with the original lesions were removed completely.

Despite the availability of numerous types of imaging agents, there is a continued need for alternative and/or improved imaging agents including those for use as contrast agents for use in the human or animal circulatory systems, including the vasculature, and other systems such as the respiratory, lymphatic, nervous, renal/urinary, reproductive and alimentary systems. In particular, there is a continued need for imaging agents that are imageable and thus visible under a range and combination of modalities and which may be used in a range of medical treatments and diagnostic methods. Many of the known imaging agents have potential limitations based for example on their physicochemical and toxicity profiles and/or their cost and availability.

The present invention is partly based on the finding that silicon, in particular, porous silicon is imageable under a broad range of modalities, including combinations thereof.

### SUMMARY OF THE INVENTION

According to a first aspect of the present invention, a method of imaging a human or animal subject is provided, wherein the contrast of the image is enhanced by administering an imaging agent comprising silicon to the human or animal subject.

Enhancement of the contrast of the image may occur through the use of positive and/or negative contrast.

According to a further aspect of the present invention, a method of diagnosis and/or monitoring, and/or treatment in a human or animal subject comprising imaging the human or animal subject is provided, wherein the contrast of the image is enhanced by administration of an imaging agent comprising silicon. The diagnosis and/or monitoring and/or treatment may typically be in relation to one or more of a disease, condition or injury. The treatment itself may be subject to the monitoring.

The imaging agent may be in a form suitable for use as a contrast agent for use in the human or animal circulatory systems and especially one or more of the vasculature, the respiratory system, the lymphatic system, the reproductive system, the renal/urinary system, the alimentary system, the nervous system, and, as such, a further aspect of the present invention provides a method of imaging the human or animal body system, including the circulatory system, for example one or more of the vasculature, the respiratory system, the lymphatic system, the renal/urinary system, the reproductive system, the alimentary system, the nervous system, wherein the contrast of the image is enhanced by administering an imaging agent comprising silicon to the human or animal subject.

The vasculature is defined as relating to the vessels that carry blood throughout the body. It includes all vessels of the body from the aorta to arteries, capillaries and draining veins. The vasculature begins and ends at the heart and its chambers and includes the various branching networks of various organ systems such as the brain, lungs and kidneys and also includes the vessels of the heart itself.

Also, the imaging agent may be in a form suitable for use as a tissue marker, for example, in the form of a pellet and as such, according to a further aspect, the present invention provides a method for tissue marking an anatomical site comprising delivery of a tissue marker comprising silicon.

In particular, the tissue marker may mark the site of a biopsy. As opposed to other contrast agents, tissue markers should move as little as possible once they have been positioned and are preferably static.

The tissue marker may be in the form of granules and/or particles. The granules and/or particles may be suspended or dispersed through a semi-solid, viscous or gelatinous carrier. The suspension and/or dispersion may be used for filling a tissue void created by a surgical procedure or for delineating a volume of tissue for treatment or for treatment monitoring.

Preferably the tissues marked include one or more of the colon, rectum, prostate, breast, brain, kidneys, liver, lungs, bone, oropharynx, skin, lymph nodes, adrenals, testis, ovaries, ureter, nerve, bladder, heart, spleen and soft tissues in general including muscles.

The imaging agent may be in the form of a positioning aid, for example, to provide an image of surgical tools that have inadvertently been left inside a patient or for surgical implants or other objects which are purposely inserted into a patient. Suitable examples include coronary or oesophageal stents, intercostal tubes, endotracheal tubes, nasogastric tubes, intravenous canulas and the like, or as part of an orthopaedic implant. The inclusion of radiopaque or ultrasound visible markings will confirm that the product has been correctly positioned during insertion.

According to a further aspect of the present invention, a pack or kit is provided, comprising an appropriate amount of an imaging agent and a separate volume of liquid or gelatinous or colloidal gel carrier, together with instructions for preparing an administrable solution or suspension of the imaging agent in the liquid or gelatinous or colloidal gel carrier, wherein the imaging agent comprises silicon. Alternatively, the imaging agent may be provided ready made up in a solution or suspension. The suspension or solution may be packaged in a vial, syringe, capsule, nasal spray, oral spray or other standard package. The imaging agent will preferably be in a form suitable for imaging with one or more of a range of modalities. For use as a tissue marker, the imaging agent may be in the form of a pellet.

The present invention also describes those methods of imaging and/or diagnosis according to the various aspects of the present invention wherein a sample is subjected to imaging outside of the human or animal body. This includes the situation wherein a sample has been removed from the human or animal body or generated outside of the human or animal body. For example, a tissue marker according to the present invention which has been used to mark a breast lesion could be removed during surgery and the surgical specimen imaged using a suitable modality, such as x-ray or ultrasound, in order to confirm the lesion was successfully removed.

According to a further aspect of the present invention, a composition comprising an imaging agent which comprises silicon for use in imaging of a human or animal body is provided.

According to a further aspect of the present invention, the use of an imaging agent comprising silicon in the manufacture of a medicament for imaging the human or animal body is provided.

According to a further aspect of the present invention, the use of silicon as an imaging agent is provided.

The present invention also describes those methods of imaging and or diagnoses wherein the imaging agent is modified in such a way in order to be suitable for use in molecular imaging of the human or animal body. Such modification may comprise combining or tagging the imaging agent with an imaging probe or specific molecule that targets a tissue of interest. This may include a combination of imaging probes or specific molecules attached to the imaging agent that target a tissue of interest.

The imaging agent is detectable when subjected to one or more of a range of modalities and, as such, the methods of imaging and/or diagnoses according to the present invention may include the use of one or more of a range of modalities. These modalities include those comprising or consisting of, for example, x-ray, CT, gamma scintigraphy, PET scintigraphy, optical imaging, fluorescence imaging, thermal imaging, infrared, ultrasound, MRI. Preferred combinations of modalities include those comprising or consisting of: CT and ultrasound; x-ray and ultrasound; CT and MRI; MRI and ultrasound; PET scintigraphy and CT; gamma scintigraphy and CT; PET scintigraphy and MRI; gamma scintigraphy and MRI. In this context, “combination” may be taken to mean, simultaneous or substantially simultaneous, or sequential image acquisition in connection with a particular treatment, or hybrid imaging where the results from two modalities are fused into a conjoint study. Fusing into a conjoint study, also referred to as hybrid imaging, utilises software and/or hardware whereby two or more imaging data sets are merged into a standard anatomical volume to provide improved localisation and/or enhanced information based on the separate finding within each imaging modality.

These modalities are also of use in that aspect of the invention which relates to a pack or kit.

The use of thermal imaging according to the present invention is considered to be of particular use when used in combination with one or more modalities the use of which gives rise to differences in the thermal conductivity and/or diffusivity of neighbouring areas of the subject or sample being imaged. Small temperature differences arising through irradiation of a subject or sample give rise to contrast when used in combination with a thermal imager. Examples of modalities which may result in suitable selective heating of the silicon imaging agent include x-rays and pulsed optical excitation. Examples of modalities which may result in suitable selective heating of the surrounding tissue include ultrasound and near infrared excitation. According to the present invention in all its aspects, the use of thermal imaging in combination with one or more modalities to detect small changes in temperature, due to the use of the one or more modalities, is also applicable to imaging agents in general and not just those comprising or containing silicon. This is particularly the case for tissue markers.

As used herein, references to the human or animal body or subject may include the whole or a part thereof.

### DETAILED DESCRIPTION OF THE INVENTION

### The Imaging Agent

The imaging agent consists of, comprises, or consists essentially of silicon.

As used herein, and unless otherwise stated, the term “silicon” refers to solid elemental silicon. For the avoidance of doubt, and unless otherwise stated, it does not include silicon-containing chemical compounds such as silica, silicates or silicones, although it may be used in combination with these materials. The silicon may be about 98 to 99.999999% pure, preferably 99 to 99.999% pure and even more preferably 99.9 to 99.999% pure.

The physical forms of silicon which are suitable for use in the present invention may be chosen from or comprise amorphous silicon, single crystal silicon and polycrystalline silicon (including nanocrystalline silicon, the grain size of which is typically taken to be 1 to 100 nm), bulk crystalline silicon and including combinations thereof. Any of the above-mentioned types of silicon, which are suitable for use in the present invention, may be porosified to form porous silicon, which may be referred to as “pSi”. The silicon may be surface porosified, for example, using a stain etch method, a gas etch method or more substantially porosified, for example, using an anodisation technique. Preferred forms of porous silicon for use in the present invention are mesoporous, microporous or macroporous silicon. Microporous silicon contains pores possessing a diameter less than 2 nm; mesoporous silicon contains pores having a diameter in the range of 2 to 50 nm; and macroporous silicon contains pores having a diameter greater than 50 nm. The use of porous silicon as an imaging agent according to the present invention is particularly useful, especially in combination with the use of x-rays, because the density of the porous silicon may be easily controlled by adjusting the porosity. The silicon may comprise, for example, a combination of bulk and porous silicon. This may be achieved by partial porosification and/or by combining the different forms of silicon.

Preferably the silicon is biodegradable or resorbable. This means the silicon dissolves over a period of time in any one of a range of physiological environments, the by-product of which is silicic acid which may be excreted by the body. The rate at which the biodegradable, or resorbable, silicon degrades will depend to some extent on the particular application and mode of administration. Biodegradability or resorbability, in particular of porous silicon, is dependent on a number of factors including the degree of porosity and/or nature of the surface modification and can therefore be tailored accordingly. The choice of excipient, if present, can also affect the rate of degradation. An example of resorbable silicon is mesoporous silicon. Another factor is the wall thickness of the silicon in the porous matrix which is generally less than a certain width in order to biodegrade. As well as being dependent on the overall porosity of the silicon, the pore morphology, i.e. the size and shape of the pores, is an important factor. In order for the silicon to be biodegradable, the silicon preferably has a surface area in the range 100 m²/g to 2600 m²/g. For mesoporous silicon tissue markers and mesoporous microparticle contrast agents suitable for use in the human or animal circulatory systems it is in the range 100 m²/g to 700 m²/g. For non-porous nanoparticle contrast agents suitable for use in the human or animal circulatory systems, the surface area is in the range 10 to 2600 m²/g. The BET surface area is determined by a BET nitrogen adsorption method as described in Brunauer et al., J. Am. Chem. Soc., 60, p 309, 1938. The BET measurement is performed using an Accelerated Surface Area and Porosimetry Analyser (ASAP 2400) available from Micromeritics Instrument Corporation, Norcross, Ga. 30093. The sample is outgassed under vacuum at 350° C. for a minimum of 2 hours before measurement.

The imaging agent suitable for use in the present invention may comprise bioactive silicon. Bioactive materials are highly compatible with living tissue and capable of forming a bond with tissue by eliciting a specific biological response. Bioactive materials may also be referred to as surface reactive biomaterials. Bioactive silicon comprises a nanostructure and such nanostructures include: (i) microporous silicon, mesoporous silicon either of which may be single crystal silicon, polycrystalline silicon or amorphous silicon; (ii) polycrystalline silicon with nanometre size grains; (iii) nanoparticles of silicon which may be amorphous or crystalline. Preferably, for use as a bioactive material, the silicon is microporous.

With regard, in particular, to the use of the modality fluorescent imaging, the photoluminescent properties of silicon (particularly nanostructured silicon) may be exploited. The structural and luminescence properties of silicon are described by Cullis et al in J. Appl. Phys., vol. 82, pp 909 to 965, 1997. The peak wavelength of emission may lie in the range corresponding to the visible and/or near-infrared, i.e. 400 nm to 1100 nm and preferably 700 nm to 1100 nm. The luminescence from the imaging agent can be excited and detected using standard medical opto-electronic equipment such as fibre-optic based endoscopes. Advantageously, therefore, the silicon may be both biodegradable and photoluminescent.

Luminescent porous silicon has been primarily studied for its applications in opto-electronics and as a sensor substrate for toxin and pathogen detection. The present invention describes the use of luminescent porous silicon as an imaging agent for in-vivo and ex-vivo optical imaging of a living organism or tissue derived from a living organism.

Porous silicon has been shown to exhibit efficient photoluminescence at room temperature across a wide spectrum, from near infra-red to ultraviolet, but particularly strong in the near infra-red (IR-band) and lower red to yellow part of the visible spectrum (S-band). IR photoluminescence at room temperature has low quantum efficiency for unoxidised porous silicon, but is much stronger when the porous silicon is oxidised, typically giving radiative efficiencies greater than 0.1%, see L. Tsybeskov et al, Phys Rev B vol. 54, 1996. S-band output emission of oxidised and unoxidised porous silicon is efficient under blue-UV photoexcitation, see J. C Vial et al, Phys Review B (USA) Vol. 45, (1992), and is weaker under infrared multiphoton excitation see J. Diener et al, Phys Review B (USA) Vol. 52 (1995). The photoluminescent decay times of emissions at the lower red end of the spectrum are typically 100-150 μs, see P. D. J. Calcott et al, J. Phys. Condens. Matter (UK), vol. 5, L91-98 (1993).

A detailed review of the basics of porous silicon photoluminescence is provided in “The Structural and Luminescence Properties of Porous Silicon”, A. G. Cullis, L. T. Canham & P. D. J. Calcott, Journal of Applied Physics August 1997. While the photoluminescence mechanism is still a source of debate, it is generally agreed that the light emission is due to the spatial confinement of electron-hole pairs in nanometre-scale silicon structures that remain after etching. Any fabrication mechanism that produces these nanostructures is therefore adequate for producing photoluminescent porous silicon.

Ideal luminescent dopants of porous silicon for the purposes of in-vivo imaging are chemi-luminescent proteins such as luciferase and aequorin, fluorescent proteins such as green fluorescent protein, yellow fluorescent protein and the like. Useful dopants are not limited to proteins but may also be selected from a range of inorganic dopants like rare earths and organic molecules such as fluorescein. Suitable dopants will be compatible with the biological system under investigation and the wavelength of the emitted luminescence will be of a nature that penetrates surrounding tissue and is able to be imaged from outside the organism.

Living tissue, in particular skin and the sub-dermal layers are relatively permeable to light in the near infra-red, especially in the wavelengths 600-1800 nm and this therefore forms the ideal range for selecting a luminescent dopant or tuning of porous silicon photoluminescence. However, emitters outside this range may also be suitable, for example luciferase, which, even at low concentrations of substrate, emits light efficiently at 486 nm and hence offsets the lower penetration of light with its higher relative strength of the light source.

Luminescent porous silicon which is suitable for use in the present invention and which is biocompatible and/or biodegradable may be prepared using any of the aforementioned techniques. Furthermore, if combined with molecular tags such as antibodies or other recognition elements, biocompatible luminescent porous silicon particles may be formed into targeted molecular imaging agents.

Luminescence derived from such particles can be detected by ex-vivo optical detection equipment. Such equipment may be designed to detect illumination from depths as much as 2-3 cm within an organism, where the scattering effect of the intervening tissue renders resolution relatively low, in the order of millimetres. This range/resolution is suitable for some forms of diagnostic imaging, including but not limited to mapping circulatory systems, locating hotspots of particular biological activity or disease, diffusion and release patterns of particles and drugs.

An example of this sort of equipment is the Xenogen IVIS Imaging System and Living Image software, by Xenogen Bioscience of Alameda, Calif., USA. The Xenogen system integrates CCD cameras, imaging chambers and software to image small animals and organs in the red region of the spectrum. Typically the animal will be dosed with systemic or targeted fluorescing agents which are allowed to accumulate in the tissue or organ of interest and then the animal is immobilised and imaged.

Much finer resolution, up to the micron range, can be obtained using optical microscopes. However, the depth of imaging is, in this case, much reduced, typically to less than 1 mm. This range/resolution is also suitable for diagnostic imaging, including but not limited to examining dermal layers, biopsy samples and internal surfaces such as the lining of the gastro-intestinal tract.

According to the present invention, these optical imaging techniques may be used alone, or in combination with other imaging modalities such as ultrasound, CT, x-ray and MRI to provide enhanced diagnostic information.

Strongly photoluminescent porous silicon can be prepared by exposing a silicon wafer to light, for example white light, whilst anodisation takes place. Variation of the light frequency and intensity, in combination with variations in etching reactants, enables ‘tuning’ of the resultant peak photoluminescent wavelength. In particular, use of lasers is desirable, because the wavelength, pattern, intensity and duration of illumination can be tightly controlled. Some examples of lasers which may be used are Nd:YAG lasers, InGaAsP/InP DFB lasers, GaAs/GaInP lasers, CO₂ lasers, diode pump solid state lasers, femtosecond (FS) lasers and picosecond (PS) lasers. The silicon need not be exposed to light for the entire period of anodisation. Depending on the final pore morphology, desired illumination may be limited to pre-anodisation, initial anodisation, intermittent illumination throughout anodisation or constant illumination throughout the anodisation process. Photoluminescent porous silicon may also be produced using the stain etch process, by pre-illumination with laser light as described in U.S. Pat. No. 6,864,190. The photoluminescence features of porous silicon may also be strongly affected by post fabrication treatments of the porous silicon surface, for example, by methanol exposure, F₂ and H₂O exposure, chloride salt treatment and thermal and chemical oxidation. Porous silicon can also be rendered luminescent by doping with photo-luminescent or chemi-luminescent substances. Such doping can be achieved by any of the aforementioned doping or surface derivatisation techniques.

The precise form and characteristics of the silicon will depend to some extent on the particular application for which it is being used. For example, the imaging agent may be administered orally, intravenously or inhalationally. If the imaging agent is being administered intravenously, it will be necessary for the silicon to be able to move through the vasculature, i.e. the blood vessels and capillaries.

The imaging agent may be in a form suitable for inhalational administration, such that particles settle on the surfaces of airways, cavities and lungs. The present invention thereby provides a method for imaging airways, cavities, lungs and any features of interest such as growths, obstructions or deformities. Inhalational administration typically involves the incorporation of particulate porous silicon which may, optionally, be loaded with one or more additives that increase the density of the particles, and/or are paramagnetic in nature and/or are positron or gamma emitters, into an aerosol formulation that is inhaled and imaged using techniques such as, but not limited to, x-ray, CT, MRI, gamma scintigraphy and/or PET scintigraphy. The aerosol formulation may be in the form of a nebulised solution with a liquid carrier incorporating the particulate porous silicon or a pressurised inhalational delivery system.

The imaging agent may further be in a form suitable for oral administration. Such a form will preferably not biodegrade or otherwise be absorbed or rendered less imageable for part of, or the entire passage through, the digestive system. Oral administration may typically involve the incorporation of particulate porous silicon which may, optionally, be loaded with one or more additives that increase the density of the particles, and/or are paramagnetic in nature and/or are positron or gamma emitters, into a non-digestible formulation that is taken orally and passes through the alimentary system. Suitable modalities for imaging the orally administered formulation include one or more of, but not limited to, x-ray, CT, ultrasound, MRI, gamma scintigraphy, PET scintigraphy. The oral formulation may be in the form of a liquid, incorporating particulate porous silicon. Alternatively, the oral formulation may be in the form of a semi-solid material incorporating particulate porous silicon. Such a semi-solid formulation may be swallowed or administered via a feeding tube (e.g. nasogastric, percutaneous endoscopic gastrotomy tube or a nasojejunal tube). The oral formulation may be in the form of particulate porous silicon incorporated into a meal that would allow imaging using a number of techniques for assessment of eating, swallowing, gastric/intestinal/colonic passage.

For use in the urinary system, the imaging agent may be administered via the use of a catheter placed into the urethra, bladder and/or ureters. Alternatively the imaging agent may be administered via the vasculature and excreted renally. Suitable modalities for imaging the intravenous or catheter administered urinary imaging formulations include one or more of, but are not limited to, x-ray, CT, MRI, ultrasound, gamma scintigraphy, PET scintigraphy. The urinary catheter based imaging formulation may be in the form of a liquid incorporating particulate porous silicon.

For use in the lymphatic system, the imaging agent will preferably be in the form of particles, said particles in the range 5 nm to 2 μm diameter, more preferably 10 to 500 nm diameter. Particles of this size are small enough to migrate through the lymphatic network but large enough to be trapped and accumulate at the lymph nodes.

The imaging agent may comprise one or more further components. For example, in order to enhance the imaging characteristics of the marker and/or the multi-modality imaging characteristics of the imaging agent, dense ions such as those comprising barium and/or iodine may be combined with the silicon. The addition of these particular components is preferred when the methods of the present invention are carried out using imaging techniques that make use of x-rays, including CT. One or more metals may be incorporated via a range of techniques including electroless plating, electroplating, co-compression or co-milling. Suitable metals include one or more of the following: cadmium, cesium, cobalt, copper, gallium, lead, manganese, molybdenum, niobium, rubidium, ruthenium, scandium, technetium, titanium, gold, tantalum, iridium, platinum, tungsten, rhodium, palladium, strontium, samarium, thallium, holmium, scandium, zirconium, yttrium, silver, iron, gadolinium, chromium, zinc, barium, magnesium, calcium, including all stable and unstable isotopes of these atoms. Other suitable materials include stainless steel. Metallic ions such as, for example, iron, manganese and gadolinium may be used in combination with the silicon for use in combination with MRI imaging systems.

Other components which may be combined with the imaging agent include non-metals such as one or more of the following: bromine; carbon; fluorine; hydrogen; iodine; nitrogen; oxygen; selenium, phosphorous, xenon, chlorine including the stable and unstable isotopes of these atoms. These and other non-metals may affect the density and imaging characteristics of the imaging agent. The various isotopes may influence MRS signature or allow imaging with PET.

Other suitable components which may be combined with the imaging agent include one or more gases. Preferred gases are inert and biocompatible, that is, they are not injurious to biological function. Any suitable biocompatible gas, gas precursor or mixture thereof may be employed, the gas being selected with regard to the chosen modality. Preferred gases may comprise, for example, one or more of the following: nitrogen; oxygen; carbon dioxide; hydrogen; nitrous oxide; a noble or inert gas such as helium, neon, argon, radon, xenon or krypton; a radioactive gas; a hyperpolarized noble gas such as hyperpolarized argon; a low molecular weight hydrocarbon; a cycloalkane; an alkene; an alkyne; an ether; a ketone; an ester; sulfur-based gases; halogenated gases, preferably fluorinated gases, including, for example, partially fluorinated gases or completely fluorinated gases such as sulphur hexafluoride, fluorohydrocarbons, perfluorocarbons, fluorocarbon gases, other fluorinated halogenated organic compounds in the gas phase, and mixtures thereof. Preferred gases also include any pharmaceutically acceptable gas mixture such as air and air/perfluorocarbon mixtures. Preferably, the perfluorocarbon gas is selected from perfluoromethane, perfluoroethane, perfluoropropane and perfluorobutanes. These and other gases may affect the density and the imaging characteristics of the imaging agent. The various isotopes may influence MRS signature or allow imaging with PET.

The further components may be combined with the silicon using a range of techniques. For example, the one or more further components may be incorporated within the pores of porous silicon or within the pores formed by the agglomeration of silicon particles. The one or more further components may be incorporated into the silicon matrix.

Metal additives can be incorporated with the silicon imaging agent by a number of means. For example, if the metal is required to be distributed uniformly and at high concentrations within a mesoporous structure, then the technique disclosed in WO 99/53898, the contents of which are hereby incorporated in their entirety, can be used. Here, a low melting point salt of the metal is drawn by capillary forces into the material whilst molten. The salt is also chosen so that on subsequent heating to a higher temperature, it decomposes to an appropriate form. If the metal is needed at low concentrations within the structure, then it can be incorporated via solutions that wet that structure and are subsequently evaporated. If the metal is required to reside predominantly on the surface of the imaging agent it can be precipitated from solutions that do not wet the mesoporous structure.

The further components may be added in an amount in the range of 0.01 to 25 wt %, preferably, 0.1 to 1 wt % of the total weight of the silicon and further component. Depending on the nature of how the further component is added to the silicon, this may affect the purity of the silicon. For example, if a metal is incorporated into the silicon lattice, this will lower the purity of the silicon and the preferred ranges of purity of silicon may be lowered accordingly to about 75 wt %.

Formulations according to the present invention may comprise, in addition to one or more imaging agents, one or more further components such as excipients. Preferred excipients are inactive substances which may be used as diluents or vehicles for active ingredients, and/or to aid the process by which a product is manufactured. An active substance may be dissolved or mixed with one or more excipients to achieve a desired formulation. Depending on the route of administration, and the desired properties and application for a formulation, various excipients may be used.

Any suitable excipient may be employed, the excipient being selected with regard to the chosen formulation, for example, solution versus tablet formulations. Excipients and their properties and applications are described generally in Rowe, R. C., Sheskey, P. and Owen, S. C. (Eds.) 2006, Handbook of Pharmaceutical Excipients, 5^{th} Ed., Pharmaceutical Press (London) and American Pharmacists Association (Washington). Excipients for liquid formulations are also discussed generally in Strickley R. G., 2004, “Solubilizing excipients in oral and injectable formulations”, Pharm. Res., 21(2): 201-30.

Coatings and integrated excipients such as polymers, starches, gelatins, and the like may be a constituent of the imaging agent. Coatings and integrated excipients may be used which enhance the biofunctionality of the imaging agent, thus affecting its biocompatibility, biodegradability, movement within a tissue or circulatory system, immuno response and metabolic/excretory pathways. The coatings and integrated excipients may also affect the structural properties of the imaging agent, acting as binders, delivery agents, lubricants, disintegrants and metabolic retardants.

Excipients can be used to solubilize, stabilize, suspend, disperse, dilute and/or emulsify the imaging agent into a liquid form, ensuring that it remains imageable and stable in suspension for the desired period of time and application, taking into account the route of administration. Preferred formulations may comprise, for example, one or more of the solubilizing agents, wetting agents, solvents, surfactants, detergents, phospholipids, and/or dissolution enhancing excipients contained in the lists set out below.

Suitable solubilizing agents may be selected from one or more of benzalkonium chloride, benzethonium chloride, benzyl alcohol, benzyl benzoate, cetylpyridinium chloride, cyclodextrins (alpha-cyclodextrin, beta-cyclodextrin, hydroxypropyl-beta-cyclodextrin, sulfobutylether-beta-cyclodextrin), glyceryl monostearate, lecithin, meglumine, macrogol 15 hydroxystearate, poloxamer, polyethylene alkyl ethers, polyoxyethylene alkyl ethers, polyoxyethylene caster oil derivatives, polyoxyethylene sorbitan fatty acid esters, polyoxyethylene stearates, povidone, 2-pyrrolidone, sodium bicarbonate, sorbitan esters (sorbitan fatty acid esters), stearic acid, hypromellose,

Suitable solvents may be selected from one or more of albumin, acetone, alcohol (ethanol), almond oil, benzyl alcohol, benzyl benzoate, canola oil, carbon dioxide, castor oil, corn oil, cottonseed oil, dibutyl phthalate, diethyl phthalate, dimethyl ether, dimethyl phthalate, dimethyl sulfoxide, dimethylacetamide, ethyl acetate, ethyl lactate, ethyl oleate, glycerin, glycofurol, isopropyl alcohol, isopropyl myristate, isopropyl palmitate, medium-chain triglycerides, mineral oil, mineral oil (light), N-methyl-2-pyrrolidone, octyldodecanol, olive oil, peanut oil, peppermint oil, polyethylene glycol (e.g. polyethylene glycol 300, polyethylene glycol 400), propylene carbonate, propylene glycol, 2-pyrrolidone, safflower oil, sesame oil, soybean oil, sunflower oil, triacetin, triethanolamine, water, hydrogenated vegetable oils, hydrogenated soybean oil, and/or medium-chain triglycerides of coconut oil and palm seed oil.

Suitable surfactants may be selected from one or more of lauric acid, triethyl citrate, anionic surfactants: docusate sodium, sodium lauryl sulfate, wax (anionic emulsifying); cationic surfactants: benzethonium chloride, cetrimide, cetylpyridinium chloride; nonionic surfactants: glyceryl monooleate, polyoxyethylene alkyl ethers, polyoxyethylene castor oil derivatives, polyoxyethylene sorbitan fatty acid esters, polyoxyethylene stearates, sorbitan esters (sorbitan fatty acid esters), wax (emulsifying), Cremophor EL, Cremophor RH 40, Cremophor RH 60, d-alpha-tocopherol polyethylene glycol 1000 succinate, polysorbate 20, polysorbate 80, Solutol HS 15, sorbitan monooleate, poloxamer 407, Labrafil M-1944CS, Labrafil M-2125CS, Labrasol, Gellucire 44/14, Softigen 767, and mono- and di-fatty acid esters of PEG 300, 400, or 1750).

Suitable organic liquids/semi-solids include one or more of beeswax, d-alpha-tocopherol, oleic acid, medium-chain mono- and diglycerides, phospholipids (hydrogenated soy phosphatidylcholine, distearoylphosphatidylglycerol, L-alpha-dimyristoylphosphatidylcholine, L-alpha-dimyristoylphosphatidylglycerol), dissolution enhancers (calcium carbonate, crospovidone, fructose, oleyl alcohol).

Preferred formulations may also comprise, for example, one or more of the emulsifying agents and/or emulsion stabilizers contained in the lists below.

Emulsifying agents include one or more of acacia, agar, ammonium alginate, calcium alginate, carbomer, carrageenan, cetostearyl alcohol, cetyl alcohol, cholesterol, diethanolamine, ethylene glycol palmitostearate, glyceryl monooleate, glyceryl monostearate, hectorite, hydroxypropyl cellulose, hydroxypropyl starch, hypromellose, lanolin, lanolin alcohols, lanolin (hydrous), lauric acid, lecithin, linoleic acid, magnesium oxide, medium-chain triglycerides, methylcellulose, mineral oil and lanolin alcohols, monoethanolamine, myristic acid, octyldodecanol, oleic acid, oleyl alcohol, palmitic acid, pectin, poloxamers, polycarbophil, polyoxyethylene alkyl ethers, polyoxyethylene caster oil derivatives, polyoxyethylene sorbitan fatty acid esters, polyoxyethylene stearates, potassium alginate, propylene glycol alginate, saponite, sodium borate, sodium citrate dehydrate, sodium lactate, sodium lauryl sulfate, sodium phosphate (monobasic), sorbitan esters (sorbitan fatty acid esters), stearic acid, sunflower oil, tragacanth, triethanolamine, wax (anionic emulsifying), wax (nonionic emulsifying), xanthan gum.

Suitable emulsion stabilizers include aluminum stearate, colloidal silicon dioxide, glyceryl monooleate, polyethylene glycol, poly(methyl vinyl ether/maleic anhydride), zinc acetate.

Preferred formulations may also comprise, for example, one or more of the stabilizing, suspending agents, and/or humectants contained in the following lists.

Suitable stabilizing agents include acacia, agar, albumin, alginic acid, aluminum stearate, ammonium alginate, ascorbic acid, ascorbyl palmitate, bentonite, butylated hydroxytoluene, calcium alginate, carboxymethylcellulose calcium, carboxymethylcellulose sodium, carrageenan, ceratonia, cyclodextrins, diethanolamine, edetates, ethylcellulose, ethylene glycol palmitostearate, glyceryl monostearate, guar gum, hydroxypropyl cellulose, hypromellose, invert sugar, lecithin, magnesium aluminum silicate, mineral oil and lanolin alcohols, monoethanolamine, pectin, polacrilin potassium, poloxamer, polyvinyl alcohol, potassium alginate, potassium chloride, povidone, propyl gallate, propylene glycol, propylene glycol alginate, raffinose, sodium acetate, sodium alginate, sodium borate, sodium stearyl fumarate, sorbitol, stearyl alcohol, sulfobutylether beta-cyclodextrin, trehalose, wax (white), wax (yellow), xanthan gum, zylitol, zinc acetate.

Suitable suspending agents include acacia, agar, alginic acid, bentonite, calcium stearate, carbomers, carboxymethylcellulose calcium, carboxymethylcellulose sodium, carrageenan, cellulose (microcrystalline), cellulose (powdered), ceratonia, colloidal silicon dioxide, dextrin, gelatin, guar gum, hydroxyethyl cellulose, hydroxyethylmethyl cellulose, hydroxypropyl cellulose, hypromellose, kaolin, magnesium aluminum silicate, maltitol solution, medium-chain triglycerides, methylcellulose, polycarbophil, polyethylene glycol, polyoxyethylene sorbitan fatty acid esters, potassium alginate, povidone, propylene glycol alginate, sesame oil, sodium alginate, sodium starch glycolate, sorbitan esters (sorbitan fatty acid esters), sucrose, tragacanth, xanthan gum.

Suitable humectants include one or more of ammonium alginate, cyclomethicone, glycerin, polydextrose, propylene glycol, sodium hyaluronate, sodium lactate, sorbitol, trehalose, triacetin, triethanolamine, xylitol.

Gel, stiff or viscous formulations may be preferred for use in the formulations according to the present invention. Such gel formulations may comprise, for example, one or more of the following gelling agents: hydrogels; stiffening agents; thickening agents; and viscosity-increasing agents.

Suitable gelling agents include avicel, aluminum stearate, calcium silicate, carbomers, carboxymethylcellulose sodium, carrageenan, chitosan, colloid silicon dioxide, gelatin, glyceryl monooleate, glyceryl palmitostearate, guar gum, hydroxyethyl cellulose, microcrystalline cellulose and carboxymethylcellulose sodium, pectin, polyethylene alkyl ethers, polyethylene glycol, polyethylene oxide, polymethacrylates, propylene carbonate, sodium ascorbate, sorbitol, zinc acetate.

Suitable hydrogels include hydroxyethyl cellulose, sodium alginate, urethane.

Suitable stiffening agents include castor oil (hydrogenated), cetyl alcohol, dextrin, paraffin, stearyl alcohol, wax (anionic emulsifying), wax (carnauba), wax (cetyl esters), wax (microcrystalline), wax (nonionic emulsifying), wax (white), wax (yellow).

Suitable thickening agents include agar, ammonium alginate, calcium alginate, colloidal silicon dioxide, dextrin, ethylcellulose, ethylene glycol palmitostearate, hydroxyethyl cellulose, hydroxyethylmethyl cellulose, hydroxypropyl cellulose, hydroxypropyl starch, hypromellose, methylcellulose, octyldodecanol, pectin, polycarbophil, polyethylene glycol, polyethylene oxide, potassium alginate, trehalose, xanthan gum, zinc stearate.

Suitable viscosity-increasing agents include acacia, agar, alginic acid, bentonite, carbomers, carboxymethylcellulose calcium, carboxymethylcellulose sodium, carrageenan, ceratonia, cetostearyl alcohol, chitosan, colloidal silicon dioxide, cyclomethicone, ethylcellulose, gelatin, glycerin, glyceryl behenate, guar gum, hectorite, hydroxyethyl cellulose, hydroxyethylmethyl cellulose, hydroxypropyl cellulose, hydroxypropyl starch, hypromellose, magnesium aluminum silicate, maltodextrin, methylcellulose, polydextrose, polyethylene glycol, poly(methyl vinyl ether/maleic anhydride), polyvinyl acetate phthalate, polyvinyl alcohol, potassium chloride, povidone, propylene glycol alginate, saponite, sodium alginate, sodium chloride, stearyl alcohol, sucrose, sulfobutylether beta-cyclodextrin, tragacanth, vegetable oil (hydrogenated), and/or xanthan gum.

Excipients commonly used in tablet or pellet formulations include binders, fillers, disintegrents, lubricants, alkalizing agents, and coatings.

Binders are excipients which hold together ingredients and increase strength in a tablet form, and can be included to control the rate and timing of table/pellet degradation and drug release. Preferred formulations may comprise, for example, one or more of the following binders: acacia, agar, alginic acid, carbomer, carboxymethylcellulose sodium, carrageenan, cellulose acetate phthalate, cellulose (microcrystalline), ceratonia, chitosan, copovidone, cottonseed oil, dextrates, dextrin, ethylcellulose, gelatin, glucose (liquid), glyceryl behenate, guar gum, hydroxyethyl cellulose, hydroxyethylmethyl cellulose, hydroxypropyl cellulose, hydroxypropyl cellulose (low-substituted), hydroxypropyl starch, hypromellose, inulin, lactose (anhydrous), lactose (monohydrate), lactose (spray-dried), magnesium aluminum silicate, maltodextrin, maltose, methylcellulose, poloxamer polycarbophil, polydextrose, polyethylene oxide, polymethacrylates, povidone, sodium alginate, starch, starch (pregelatinized), stearic acid, sucrose, sugar (confectioner's), sunflower oil, vegetable oil (hydrogenated), zein, brazil wax, cocoa butter, tapioca starch (cassava flour), polyethylene glycol, polymers of pyrrolidone, gelatin/glycerine mix, polyvinyl alcohols, poly(lactic-co-glycolic acid), polylactic acid.

Tablet and/or capsule fillers (diluents) are excipients which fill out the size and shape of a tablet or capsule making it practical to produce and convenient for consumer use. Preferred formulations may comprise, for example, one or more of the following fillers: ammonium alginate, calcium carbonate, calcium phosphate (dibasic anhydrous), calcium phosphate (dibasic dehydrate), calcium phosphate (tribasic), calcium sulfate, cellulose (microcrystalline), cellulose (powdered), cellulose (silicified microcrystalline), cellulose acetate, dextrates, dextrin, dextrose, erythritol, ethylcellulose, fructose, fumaric acid, glyceryl palmitostearate, isomalt, kaolin, lactitol, lactose (anhydrous), lactose (monohydrate), lactose (spray-dried), magnesium carbonate, magnesium oxide, maltodextrin, maltose, mannitol, medium-chain triglycerides, polydextrose, polymethacrylates, simethicone, sodium alginate, sodium chloride, sorbitol, starch, starch (pregelatinized), starch (sterilizable maize), sucrose, sugar (compressible), sugar (confectioner's), sugar spheres, sulfobutylether beta-cyclodextrin, talc, tragacanth, trehalose, vegetable oil (hydrogenated), xylitol, soybean oil and/or safflower oil.

Tablet and/or capsule disintegrants are excipients which expand or dissolve readily when wet, causing a tablet or pellet formulation to break apart. Importantly, disintegrants can be used to influence the longevity of tissue marker pellet formulations when placed within biological systems. Preferred formulations may comprise, for example, one or more of the following disintegrants: alginic acid, calcium alginate, carboxymethylcellulose calcium, carboxymethylcellulose sodium, cellulose (microcrystalline), cellulose (powdered), chitosan, colloidal silicon dioxide, croscarmellose sodium, crospovidone, docusate sodium, guar gum, hydroxypropyl cellulose (low-substituted), hydroxypropyl starch, magnesium aluminum silicate, methylcellulose, polacrilin potassium, povidone, sodium alginate, sodium starch glycolate, starch, starch (pregelatinized).

Lubricants prevent ingredients from clumping together and from sticking to manufacturing equipment. Preferred formulations may comprise, for example, one or more of the following lubricants: calcium stearate, castor oil (hydrogenated), glycerin monostearate, glyceryl behenate, glyceryl monostearate, glyceryl palmitostearate, magnesium lauryl sulfate, magnesium stearate, medium-chain triglycerides, mineral oil (light), myristic acid, palmitic acid, poloxamer, polyethylene glycol, potassium benzoate, sodium benzoate, sodium chloride, sodium lauryl sulfate, sodium stearyl fumarate, stearic acid, talc, vegetable oil (hydrogenated), zinc stearate, minerals, and/or silica.

Alkalizing agents can be used to increase the pH of the environment surrounding a pellet or tablet form, influencing the rate of degradation of the imaging agent. Preferred formulations may comprise, for example, one or more of the following alkalizing agents: ammonia solution, diethanolamine, monoethanolamine, potassium bicarbonate, potassium citrate, potassium hydroxide, sodium bicarbonate, sodium borate, sodium citrate dehydrate, sodium hydroxide, triethanolamine, and/or sodium phosphate dibasic.

Preferred formulations may comprise, for example, one or more of the following glidants: calcium phosphate (tribasic), calcium silicate, cellulose (powdered), colloidal silicon dioxide, magnesium oxide, magnesium silicate, magnesium trisilicate, silicon dioxide, starch, and/or talc.

Preferred formulations may comprise, for example, one or more of the following adhesives, bioadhesives and/or mucoadhesives: carbomer, chitosan, ethylcellulose backing membranes, glyceryl monooleate, polycarbophil, polyethylene oxide, and/or poly(methyl vinyl ether/maleic anhydride).

Coatings are excipients commonly used to protect active ingredients from deterioration by moisture, make oral tablets easier to swallow, improve biocompatibility, and/or control the rate and timing of drug release. Coatings may also be used to provide colour, a smooth finish, to facilitate printing on the tablet, or to flavour oral formulations. Preferred formulations may comprise, for example, one or more coatings. Suitable coatings include acetyltributyl citrate, acetyltriethyl citrate, aliphatic polyesters, calcium carbonate, carbomers, carboxymethylcellulose sodium, cellulose acetate, cellulose acetate phthalate, cetyl alcohol, chitosan, ethylcellulose, fructose, gelatin, glycerin, glyceryl behenate, glyceryl palmitostearate, guar gum, hydroxyethyl cellulose, hydroxyethylmethyl cellulose, hydroxypropyl cellulose, hypromellose, hypromellose acetate succinate, hypromellose phthalate, isomalt, latex particles, maltitol, maltodextrin, methylcellulose, poloxamer, polydextrose, polyethylene glycol, polymethacrylates, polyvinyl acetate phthalate, polyvinyl alcohol, potassium chloride, povidone, shellac, shellac with stearic acid, sucrose, sureteric, titanium dioxide, titanium oxide, tributyl citrate, triethyl citrate, vanillin, wax (carnauba), wax (microcrystalline), wax (white), wax (yellow), xylitol, zein, and/or corn protein.

Preferred formulations may also comprise, for example, one or more film forming agents. Suitable film forming agents include ammonium alginate, calcium carbonate, chitosan, chlorpheniramine maleate, copovidone, dibutyl phthalate, dibutyl sebacate, diethyl phthalate, dimethyl phthalate, ethyl lactate, ethylcellulose, gelatin, glucose (liquid), hydroxyethyl cellulose, hydroxypropyl cellulose, hypromellose, hypromellose acetate succinate, maltodextrin, polydextrose, polyethylene glycol, polyethylene oxide, polymethacrylates, poly(methyl vinyl ether/maleic anhydride), polyvinyl acetate phthalate, triethyl citrate, and/or vanillin.

Preferably, the biocompatible coating may be chemically linked to multiple sites, for example, surface groups, on the silicon. More than one biocompatible coating may be chemically linked to the native silicon particle to form more than one coat or layer or cage on the particle.

Preferably, the biocompatible coating comprises or consists of a polymer, including natural polymers, or synthetic polymers, or derivatives of each. The polymer may be grafted, linear, branched or arborized/dendrimerized. Examples of natural polymers include polysaccharides such as dextran, proteins, such as albumin, peptides and polyamino acids, such as polylysine. A synthetic polymer is obtained from nonbiological syntheses, by using standard polymer chemistry techniques known to those in the art to react monomers into polymers. The polymers may be homopolymers, (i.e., synthesized from a single type of monomer), or co-polymers, (i.e., synthesized from two or more types of monomers). The polymers can be crosslinked (e.g., a polymer in which functional groups on a polymer chain and/or branches have reacted with functional groups on another polymer to form polymer networks) or non-cross-linked (e.g., few or no individual polymer chains have reacted with the functional groups of another polymer chain to form the interconnected polymer networks). Synthetic, biocompatible polymers are discussed generally in Holland et al., “Biodegradable Polymers,” Advances in Pharmaceutical Sciences 6: pages 101-164, 1992, and U.S. Pat. No. 5,593,658. Preferred polymers have a molecular weight of about 5,000-10,000 daltons. The polymers may be attached directly to the silicon, or attached to coating agents through reactive groups on the coating agents. Alternatively, the polymers may be formed in situ, i.e., added as monomers to the fluorescent silicon nanoparticle solution, e.g. as an acrylate, and polymerized e.g., with standard polymerization chemistries, to form the polymer in the presence of the silicon particles.

Useful types of polymers include polypeptides, polyamino acids, diaminocarboxylate, copolymers, polyethyleneamines, polysaccharides, aminated polysaceharides, aminated oligosaceharides, polyamidoamines, polyacrylic acids, polyalcohols, polyoxyethylene sorbitan esters, polyoxyethylene and polyoxypropylene derivatives, polyoxyl stearates, polycaprolactones, polyanhydrides, polyalkylcyanoacrylates, polyglycerol surfactants, polycaprolactones, polyarihydrides, polymethylmethacrylate polymers, starch derivatives, dextran and derivatives thereof (i.e., carboxydextran, carboxymethyldextran, reduced carboxymethyldextran), fatty acids, their salts and derivatives, mono-, di-, and triglycerides and their derivatives, and poly-carboxylie acids. Preferred polymers include polyethylene oxide, poly(vinyl pyrrolidone), poly(methacrylie acid), poly(acrylic acid), poly(hydroxyethylmethacrylate, poly(vinyl alcohol) and natural polymers such as dextran.

The imaging agent may be encapsulated. Hence the formulations of the present invention may comprise one or more encapsulating agents. Capsules are solid dosage forms in which the drug substance and appropriate pharmaceutical adjuncts or excipients, such as fillers, binders, diluents, disintegrants, lubricants and glidants, are enclosed in a small soluble shell. Preferred encapsulating agents are inactive substances. The active substance may be enclosed by one or more encapsulating agents to achieve a desired formulation. Depending on the route of administration, and the desired properties and application for a formulation, various encapsulating agents may be used.

Any suitable encapsulating agent may be employed, the agent being selected with regard to the chosen application, for example oral versus parenteral formulations.

Encapsulating agents can be used to enclose the imaging agent, ensuring that it remains imageable and contained for the desired period of time and application, Including route of administration. Preferred formulations may comprise, for example, one or more of the following agents: gelatin, hydroxypropylmethylcellulose (HPMC), starch and cellulose acetate phthalate (CAP). These primary agents may be combined with one or more of the following: a plasticizer, water, preservatives, colorants and opacifying agents, flavourings, sugars, acids and medications.

Plasticizers are components of film coating solutions which act to make the film more pliable and enhance spread of coat. Preferred formulations may comprise, for example, one or more of the following plasticising agents: acetylated monoglyceride; butyl phthalybutyl glycolate; dibutyl tartrate; diethyl phthalate; dimethyl phthalate; ethyl phthalylethyl glycolate; sorbitol glycerin; propylene glycol; triacetin; triacetin citrate; tripropionin; acetyltributyl citrate; acetyltriethyl citrate; benzyl benzoate; chlorobutanol; dextrin; dibutyl phthalate; dibutyl sebacate; glycerine; glycerin monstearate; mannitol; mineral oil and lanolin alcohols; palmitic acid; petrolatum and lanolin alcohols; polyethylene glycol; polyvinyl acetate phthalate; 2-pyrrolidone; sorbitol; tributyl citrate; triethanolamine and/or triethyl citrate.

Excipients may also be used in combination with the active ingredient contained in a capsule and may include binders, fillers, disintegrants, lubricants, alkalizing agents, and coatings, as described above.

Other excipients that may be employed include flavours, colours, opacifying agents, and/or preservatives added to make oral tablet or liquid formulations more palatable, or improve the appearance of a formulation. Preferred formulations may comprise, for example, one or more of the following flavour enhancers, flavouring agents, taste masking agents, sweetening agents, and/or acidulents.

Flavour enhancers include acesulfame potassium, aspartame, citric acid monohydrate, dibutyl sebacate, ethyl maltol, ethylcellulose, fructose, maltol, monosodium glutamate, neohesperidin dihydrochalcone, saccharin, saccharin sodium, sodium cyclamate, tartaric acid, thaumatin, trehalose, xylitol.

Flavouring agents include denatonium benzoate, dibutyl sebacate, ethyl acetate, ethyl lactate, ethyl maltol, ethyl vanillin, ethylcellulose, fumaric acid, leucine, malic acid, maltol, menthol, phosphoric acid, propionic acid, propylene glycol alginate, sodium acetate, sodium lactate, sodium proprionate, sugar (confectioner's), thymol, triethyl citrate, vanillin.

Taste masking agents include erythritol, glyceryl palmitostearate.

Suitable sweetening agents include one or more of acesulface potassium, alitame, aspartame, dextrose, erythritol, fructose, glucose (liquid), glycerin, inulin, isomalt, lactitol, maltitol, maltitol solution, maltose, mannitol, neohesperidin dihydrochalcone, polydextrose, saccharin, saccharin sodium, sodium cyclamate, sorbitol, sucralose, sucrose, sugar (compressible), sugar (confectioner's), thaumatin, trehalose, xylitol.

Acidulents include fumaric acid, lactic acid, malic acid, phosphoric acid, sodium phosphate (monobasic), tartaric acid.

Preferred formulations may comprise, for example, one or more of the following colouring and/or pigment agents: BEIGE P-1437, BLACK LB-1171, BLACK LB-442, BLACK LB-636, BLACK LB-9972, BLACK OXIDE, BLUE #1, BLUE #1 LAKE, BLUE #2, BLUE LAKE BLEND LB-332, BLUE LAKOLENE, BLUE LB-781, BROWN LAKE, BROWN LAKE BLEND, BROWN LAKE BLEND LB-1685, BROWN LB-292, BROWN LB-464, BURNT UMBER, CARAMEL 105, CARAMEL ACID PROOF 100, CARMINE 09349, CASING 27-75, CHROMA-TERIC DEB-5037-ORE, CHROMA-TERIC T3000-WE, CHROMA-TERIC YELLOW T3277-YE, CHROMA-TONE, CHROMA-TONE PDDB-8906W, CHROMA-TONE-P DDB-8746-OR, DC BLACK #1, DC BLUE #1, DC BLUE #1 LAKE, DC BLUE #2 LAKE, DC BLUE #6, DC GREEN #1 LAKE, DC GREEN #3 LAKE, DC GREEN #4, DC GREEN #5, DC ORANGE #3, DC RED #19, DC RED #2 LAKE, DC RED #21 LAKE, DC RED #22, DC RED #27, DC RED #27 LAKE, DC RED #28, DC RED #28 LAKE, DC RED #3 LAKE, DC RED #30, DC RED #30 LAKE, DC RED #33, DC RED #33 LAKE, DC RED #36, DC RED #39, DC RED #4 LAKE, DC RED #40, DC RED #40 LAKE, DC RED #5, DC RED #6, DC RED #6 BARIUM LAKE, DC RED #6 LAKE, DC RED #7, DC RED #7 CALCIUM LAKE, DC RED #7 LAKE, DC RED LAKE, DC RED LB #9570, DC RED LB WJ-9570, DC VIOLET #2 LAKE, DC YELLOW #10, DC YELLOW #10 HT LAKE, DC YELLOW #10 LAKE, DC YELLOW #5, DC YELLOW #5 LAKE, DC YELLOW #6, DC YELLOW #6 LAKE, DIOLACK 00F32892 YELLOW, EMERALD GREEN LB, EMERALD GREEN LB-9207, FDC BLACK LB260, FDC BLUE #1, FDC BLUE #1H.T. ALUMINUM LAKE, FDC BLUE #1 LAKE, FDC BLUE #10, FDC BLUE #2, FDC BLUE #2 HT LAKE, FDC BLUE #2 LAKE, FDC BLUE #40 HT LAKE, FDC BROWN R LB-56069, FDC GREEN #1, FDC GREEN #1 LAKE, FDC GREEN #3, FDC GREEN LB-1174, FDC GREEN LB-3323, FDC GREEN LB-9583, FDC LB483, FDC ORANGE #2, FDC ORANGE LB-452, FDC PURPLE LB588, FDC PURPLE LB-694, FDC RED #1, FDC RED #19, FDC RED #2, FDC RED #2 LAKE, FDC RED #27 LAKE, FDC RED #27 LAKE, FDC RED #28, FDC RED #3, FDC RED #3 LAKE, FDC RED #30 LAKE, FDC RED #33, FDC RED #4, FDC RED #40, FDC RED #40 AC LAKE, FDC RED #40 LAKE, FDC RED #7 LAKE, FDC VIOLET #1, FDC VIOLET #1 LAKE, FDC YELLOW #1, FDC YELLOW #10, FDC YELLOW #10 LAKE, FDC YELLOW #3, FDC YELLOW #5, FDC YELLOW #5 LAKE, FDC YELLOW #6, FDC YELLOW #6 HT LAKE, FDC YELLOW #6 LAKE, FERRIC OXIDE ORANGE, GRAY #2982, GREEN 70363, GREEN AL LB-265, GREEN ALUMINUM LB, GREEN LAKE BLEND LB-1236, GREEN LAKE BLEND LB-333, GREEN LB, GREEN LB-1594, GREEN LB-1616, GREEN LB-279, GREEN LB-482, GREEN LB-555, GREEN LB-603, GREEN LB-820, GREEN LB-883, GREEN PB-1543, GREEN PB-1766, GREEN PMS-579, GREEN PR-1333, GREEN PR-1339, LAVENDER, LAVENDER LB-1356, MINT GREEN, OCHRE 3506, ORANGE LB-1387, ORANGE LB-715, PEACH LB-1576, PINK, PURPLE LAKE, PURPLE LB-1902, PURPLE LB-562, PURPLE LB-639, PURPLE LB-694, RED #27 ALUMINUM LAKE, RED #3 LAKE HT, RED #33, RED #40 ALUMINUM LAKE, RED COTOLENE-P, RED PB-1595, SALMON LB-1668, SPECTRASPRAY BLUE 50726, SWEDISH ORANGE #2191, TAN PB-1388, TAN PB-1388, TETRAROME ORANGE, TURQUOISE LB-1430, WHITE COATERIC YPA-6-7089, WHITE COTOLENE-P, WHITE TC-1032, WILD CHERRY 7598, YELLOW #10, YELLOW #10 LAKE, YELLOW #5 LAKE, YELLOW #6, YELLOW #62, YELLOW 70362, YELLOW LB 104, YELLOW LB 9706, YELLOW LB-111, YELLOW LB-1577, YELLOW LB-1637, YELLOW OCHRE, YELLOW PB1345, YELLOW PB-1381, YELLOW WD-2014, DC BLUE #4, DC BLUE #4 LAKE, DC BLUE #9, DC GREEN #5 LAKE, DC GREEN #8, DC GREEN #6, DC GREEN #6 LAKE, DC ORANGE #10, DC ORANGE #10 LAKE, DC ORANGE #11, DC ORANGE #11 LAKE, DC ORANGE #4, DC ORANGE #4 LAKE, DC ORANGE #5, DC ORANGE #5 LAKE, DC RED #17, DC RED #17 LAKE, DC RED #21, DC RED #22 LAKE, DC RED #31, DC RED #31 LAKE, DC RED #34, DC RED #34 LAKE, DC RED #36 LAKE, DC VIOLET #2, DC YELLOW #11, DC YELLOW #7, DC YELLOW #7 LAKE, DC YELLOW #8, DC YELLOW #8 LAKE, FDC GREEN #3 LAKE, FDC RED #4 LAKE, EXT. DC YELLOW #7, EXT. DC YELLOW #7 LAKE, DC LAKES, FDC LAKES, EXT. DC LAKES, E100 curcumin, E101 riboflavin, E102 tartrazine, E104 quinoline yellow, E110 sunset yellow FCF, E120 carmine, E122 carmoisine, E123 amaranth, E124 ponceau 4R, E127 erythrosine, E129 allura red AC, E131 patent blue V, E132 indigo carmine, E133 brilliant blue FCF, E140 chlorophylls, E141 copper complexes of chlorophylls and chlorophyllins, E142 green S, E150 caramel, E151 brilliant black BN, E153 vegetable carbon, E160 carotenoids, E161 xanthophylls, E162 beetroot red, E163 anthocyanins, E170 calcium carbonate, E171 titanium dioxide, E172 iron oxides and hydroxides, E173 aluminium, alumina, aluminium powder, annatto extract, beta-carotene, bismuth oxychloride, bronze powder, calcium carbonate, canthaxanthin, caramel, chromium hydroxide green, chromium oxide green, chromium-cobalt-aluminum oxide, cochineal extract (carmine), copper powder, dihydroxyacetone, ferric ammonium citrate, ferric ferrocyanide, guanine, iron oxides synthetic, logwood extract, mica, potassium sodium copper chlorophyllin, pyrogallol, pyrophyllite, talc, titanium dioxide, zinc oxide.

Preferred formulations may comprise, for example, one or more of the following opacifying agents: aluminum stearate, calcium carbonate, ethylene glycol palmitostearate, titanium dioxide, zinc acetate.

Preferred formulations may comprise, for example, one or more of the following preservatives: alcohol (ethanol), benzalkonium chloride, benzethonium chloride, benzoic acid, benzyl alcohol, boric acid, bronopol, butylated hydroxyanisole, butylparaben, carbon dioxide, cetrimide, cetylpyridinium chloride, chlorbutanol, chlorhexidine, chlorobutanol, chlorocresol, chloroxylenol, cresol, dimethyl ether, ethylparaben, glycerin, hexetidine, imidurea, isopropyl alcohol, lactic acid, methylparaben, monothioglycerol, parabens, phenol, phenoxyethanol, phenylethyl alcohol, phenylmercuric acetate, phenymercuric borate, phenylmercuric nitrate, potassium benzoate, potassium metabisulfite, potassium sorbate, propionic acid, propyl gallate, propylene glycol, propylparaben, sodium acetate, sodium benzoate, sodium borate, sodium lactate, sodium metabisulfite, sodium propionate, sodium sulfite, sorbic acid, thimerosal, and/or xylitol.

Further, delivery systems for the imaging agents may employ carrier substances or vehicles. Such carrier vehicles for delivery of the present invention may comprise, for example, one or more of the gelling agents listed above, propellants (butane, carbon dioxide, chlorodifluoroethane (HCFC), chlorodifluoromethane, chlorofluorocarbons (CFC), difluoroethane (HFC), dimethyl ether, heptafluoropropane (HFC), hydrocarbons (HC), isobutene, nitrogen, nitrous oxide, propane, tetrafluoroethane (HFC)), diluents for dry-powder formulations (monohydrated lactose and mannitol).

The manufacturing process for the imaging agent may employ the use of excipients, for example, one or more of the following: lubricants (canola oil, codliver oil, hydroxyethyl cellulose, lauric acid, leucine, mineral oil, octyldodecanol, poloxamers, polyvinyl alcohol, sodium hyaluronate, talc), air displacement agents (carbon dioxide, nitrogen), freeze-drying agents and cryoprotectants (albumin, lactose (anhydrous), mannitol, sodium bicarbonate, trehalose), sterilisation/disinfectant/antiseptic/antibacterial/antifungal/antivirals agents, and/or polishing agents (e.g. yellow wax).

Additionally, a chosen imaging agent formulation may also comprise one or more acidifying agents, adsorbents, alcohol denaturants, antiadherent agents, anticaking agents, antifoaming agents, antioxidants, buffering agents, chelating agents, dispersing agent, emollient, esterifying agent, penetration enhancers, sequestering agents, water-absorbing agents, and/or water-repelling agents. These excipients are described generally in Rowe, R. C., Sheskey, P. and Owen, S. C. (Eds.) 2006, Handbook of Pharmaceutical Excipients, 5^{th} Ed., Pharmaceutical Press (London) and American Pharmacists Association (Washington).

In PCT/GB96/01863, the contents of which are incorporated herein by reference in their entirety, it is described how bulk crystalline silicon can be rendered porous by partial electrochemical dissolution in hydrofluoric acid based solutions. This etching process generates a silicon structure that retains the crystallinity and the crystallographic orientation of the original bulk material. Hence, the porous silicon formed is a form of crystalline silicon. Broadly, the method involves anodising, for example, a heavily boron doped CZ silicon wafer in an electrochemical cell which contains an electrolyte comprising a 20% solution of hydrofluoric acid in an alcohol such as ethanol, methanol or isopropylalcohol (IPA). Following the passing of an anodisation current with a density of about 50 mAcm⁻², a porous silicon layer is produced which may be separated from the wafer by increasing the current density for a short period of time. The effect of this is to dissolve the silicon at the interface between the porous and bulk crystalline regions.

Porous silicon may also be made using a gas-etch method such as described in ‘Improved surface sensing of DNA on gas-etched porous silicon’, D. C. Tessier et al, Sensors & Actuators B, February 2006. This method involves first cleaning the silicon with RCA-type hydrogen peroxide mixtures and then partially oxidising the silicon surface by etching for several minutes with 5% HF, rinsing with water and then exposing to a flow of ozone gas (for example, 3 SLM 280 g/m³ in nitrogen). Once prepared, the silicon is loaded into the gas-etching chamber where it is exposed to a mixture of O₂, NO₂ and HF acid vapours. Silicon wafers may simply be placed on a tray and their upper surfaces exposed to the etching vapours, silicon particles may be agitated and kept airborne by use of airflows to permit gas-etching of the complete particle surface. Once a suitable etch depth and porosity is attained, the silicon is removed from the etching chamber.

Porous silicon may also be made using the so-called stain-etching technique which is another conventional method for making porous silicon. This method involves the immersion of a silicon sample in a hydrofluoric acid solution containing a strong oxidising agent. No electrical contact is made with the silicon, and no potential is applied. The hydrofluoric acid etches the surface of the silicon to create pores. The pore morphology may be tailored according to the particular application. For example, by creating pores with particularly small diameters at the surface which significantly broaden as they penetrate the silicon then this may provide useful ultrasound characteristics through, for example, the retention of gas inside the structure.

Silicon pore morphology (pore size, depth, shape, orientation) has been shown to be important in respect of porous silicon's chemistry and physical properties, in particular its biodegradability, loading capability. The various etching techniques, which include anodisation, stain etch and gas etch, all produce inherently different pore morphologies and surface features which can be tuned by varying sample preparation and etch conditions. Such variations may include varying the concentrations of the reactants, etching time, current densities, agitation of silicon particles and sample preparation such as cleaning, partial oxidation or reduction of the surface, or patterning with etch resistant films.

Following its formation, the porous silicon may be dried. For example, it may be supercritically dried as described by Canham in Nature, vol. 368, (1994), pp 133-135. Alternatively, the porous silicon may be freeze dried or air dried using liquids of lower surface tension than water, such as ethanol or pentane, as described by Bellet and Canham in Adv. Mater, 10, pp 487-490, 1998.

Silicon hydride surfaces may, for example, be generated by stain etch or anodisation methods using hydrofluoric acid based solutions. When the silicon prepared, for example, by electrochemical etching in HF based solutions, comprises porous silicon, the surface of the porous silicon may or may not be suitably modified in order, for example, to improve the stability of the porous silicon. The surfaces of the porous silicon may therefore be modified to provide: silicon oxide surfaces wherein the porous silicon may typically be described as being partially oxidised; or derivatised surfaces which may possess Si—O—C bonds and/or Si—C bonds. The surface of the porous silicon includes exterior and/or interior surfaces within the pores.

Silicon oxide surfaces may be produced by subjecting the silicon to chemical oxidation, photochemical oxidation or thermal oxidation, as described for example in Chapter 5.3 of “Properties of Porous Silicon” (edited by L. T. Canham, IEE 1997). PCT/GB02/03731, the entire contents of which are incorporated herein by reference, describes how porous silicon may be partially oxidised in such a manner that the sample of porous silicon retains some porous silicon in an unoxidised state. For example, PCT/GB02/03731 describes how, following anodisation in 20% ethanoic HF, the anodised sample was partially oxidised by thermal treatment in air at 500° C. to yield a partially oxidised porous silicon sample.

Following partial oxidation, the silicon particles may possess an oxide content corresponding to between about one monolayer of oxygen and a total oxide thickness of less than or equal to about 4.5 nm covering the entire silicon skeleton. The porous silicon may have an oxygen to silicon atomic ratio between about 0.04 and 2.0, and preferably between 0.60 and 1.5. Oxidation may occur in the pores and/or on the external surface of the silicon.

Derivatised porous silicon is porous silicon possessing a covalently bound monolayer on at least part of its surface. The monolayer typically comprises one or more organic and/or inorganic groups that are bonded by, for example, hydrosilylation to at least part of the surface of the porous silicon. Derivatised porous silicon is described in PCT/GB00/01450, the contents of which are incorporated herein by reference in their entirety. PCT/GB00/01450 describes derivatisation of the surface of silicon using methods such as hydrosilyation in the presence of a Lewis acid. In that case, the derivatisation is effected in order to block oxidation of the silicon atoms at the surface and so stabilise the silicon. Methods of preparing derivatised porous silicon are known to the skilled person and are described, for example, by J. H. Song and M. J. Sailor in Inorg. Chem. 1999, vol 21, No. 1-3, pp 69-84 (Chemical Modification of Crystalline Porous Silicon Surfaces).

Derivitisation of the silicon may be desirable when it is required to increase the hydrophobicity of the silicon, thereby decreasing its wettability. Derivatised surfaces may be modified with one or more alkyne groups. Alkyne derivatised silicon may be derived from treatment with acetylene gas, for example, as described in “Studies of thermally carbonized porous silicon surfaces” by J. Salonen et al in Phys Stat. Solidi (a), 182, pp 123-126, (2000) and “Stabilisation of porous silicon surface by low temperature photoassisted reaction with acetylene”, by S. T. Lakshmikumar et al in Curr. Appl. Phys. 3, pp 185-189 (2003).

Derivitisation of the silicon may also be desirable when it is required to increase the hydrophillicity of the silicon, thereby improving wettability and ease of dispersion in aqueous formulations. Such derivitised surfaces are described in “Carboxyl functionalization of ultrasmall silicon nanoparticles through thermal hydrosilylation” by Rogozhina et al. Journal of Materials Chemistry 16, 1421-1430 (2006).

The purity of the elemental silicon will be altered following modification such as doping, derivitisation or coating. Derivatisation, including surface oxidation, alkylation, silanisation may enhance the biofunctionality of the imaging agent thereby modifying its biocompatibility, biodegradability, movement within a tissue or circulatory system, immuno response and metabolic/excretory pathways.

The imaging agent may be administered in particulate form. Methods for making silicon powders such as silicon microparticles and silicon nanoparticles are well known in the art. These are often referred to as “bottom-up” methods, which include, for example, chemical synthesis or gas phase synthesis. Alternatively, so-called “top-down” methods refer to such known methods as electrochemical etching or comminution (e.g. milling as described in Kerkar et al. J. Am. Ceram. Soc., vol. 73, pages 2879-2885, 1990.). PCT/GB02/03493 and PCT/GB01/03633, the contents of which are incorporated herein by reference in their entirety, describe methods for making particles of silicon, said methods being suitable for making silicon for use in the present invention. Such methods include subjecting silicon to centrifuge methods, or grinding methods. Porous silicon powders may be ground between wafers or blocks of crystalline silicon. Since porous silicon has lower hardness than bulk crystalline silicon, and crystalline silicon wafers have ultrapure, ultrasmooth surfaces, a silicon wafer/porous silicon powder/silicon wafer sandwich is a convenient means of achieving for instance, a 1-10 μm particle size from much larger porous silicon particles derived, for example, via anodisation. Porous silicon particles may also be formed by sonification where sound waves of sufficient frequency and amplitude are directed at porous silicon membranes causing the membranes to fragment into particles. US 20050042764 and US 20030170162, the contents of which are incorporated herein by reference in their entirety, describe the fabrication of porous silicon particles by sonification. Porous silicon particles may also be formed from commercially available silicon powders by anodisation, stain etch or gas etch techniques.

The surface of silicon particles prepared by “top down” or “bottom up” methods may also be a hydride surface, partially oxidised, fully oxidised or derivatised. Milling in an oxidising medium such as water or air will result in silicon oxide surfaces. Milling in an organic medium may result in, at least partial derivatisation of the surface. Gas phase synthesis, such as from the decomposition of silane, will result in hydride surfaces.

Particle size distribution measurements, including the mean particle size (d₅₀/μm) of the silicon particles are measured using a Malvern Particle Size Analyzer, Model Mastersizer, from Malvern Instruments. A helium-neon gas laser beam is projected through a transparent cell which contains the silicon particles suspended in an aqueous solution. Light rays which strike the particles are scattered through angles which are inversely proportional to the particle size. The photodetector array measures the quantity of light at several predetermined angles. Electrical signals proportional to the measured light flux values are then processed by a microcomputer system, against a scatter pattern predicted from theoretical particles as defined by the refractive indices of the sample and aqueous dispersant to determine the particle size distribution of the silicon.

Other examples of methods suitable for making silicon nanoparticles include evaporation and condensation in a subatmospheric inert-gas environment. Various aerosol processing techniques have been reported to improve the production yield of nanoparticles. These include synthesis by the following techniques: combustion flame; plasma; laser abalation; chemical vapour condensation; spray pyrolysis; electrospray and plasma spray. Preferred nanoparticle synthesis techniques include: high energy ball milling; gas phase synthesis; plasma synthesis; chemical synthesis; sonochemical synthesis.

High energy ball milling, which is a common top-down approach for nanoparticle synthesis, has been used for the generation of magnetic, catalytic, and structural nanoparticles, see Huang, “Deformation-induced amorphization in ball-milled silicon”, Phil. Mag. Lett., 1999, 79, pp 305-314. The technique, which is a commercial technology, has traditionally been considered problematic because of contamination problems from ball-milling processes. However, the availability of tungsten carbide components and the use of inert atmosphere and/or high vacuum processes has reduced impurities to acceptable levels. Particle sizes in the range of about 0.1 to 1 μm are most commonly produced by ball-milling techniques, though it is known to produce particle sizes of about 0.01 μm. Ball milling can be carried out in either “dry” conditions or in the presence of a liquid, i.e. “wet” conditions. For wet conditions, typical solvents include water or alcohol based solvents.

Silane decomposition provides a very high throughput commercial process for producing polycrystalline silicon granules. Fine silicon powders are commercially available. For example, NanoSi™ Polysilicon is commercially available from Advanced Silicon Materials LLC and is a fine silicon powder prepared by decomposition of silane in a hydrogen atmosphere. The particle size is 5 to 500 nm and the BET surface area is about 25 m²/g. This type of silicon has a strong tendency to agglomerate, reportedly due to hydrogen bonding and Van der Waals forces. This agglomeration results in a high surface area form of silicon which is useful for the loading of materials therein in a similar manner as porous silicon is when produced by known, for example, electrochemical techniques.

Plasma synthesis is described by Tanaka in “Production of ultrafine silicon powder by the arc plasma method”, J. Mat. Sci., 1987, 22, pp 2192-2198. High temperature synthesis of a range of metal nanoparticles with good throughput may be achieved using this method. Silicon nanoparticles (typically 10-100 nm diameter) have been generated in argon-hydrogen or argon-nitrogen gaseous environments using this method.

Solution growth of ultra-small (<10 nm) silicon nanoparticles is described in US 20050000409, the contents of which are incorporated herein in their entirety. This technique involves the reduction of silicon tetrahalides such as silicon tetrachloride by reducing agents such as sodium napthalenide in an organic solvent. The reactions lead to a high yield at room temperature.

In sonochemistry, an acoustic cavitation process can generate a transient localized hot zone with extremely high temperature gradient and pressure. Such sudden changes in temperature and pressure assist the destruction of the sonochemical precursor (e.g., organometallic solution) and the formation of nanoparticles. The technique is suitable for producing large volumes of material for industrial applications. Sonochemical methods for preparing silicon nanoparticles are described by Dhas in “Preparation of luminescent silicon nanoparticles: a novel sonochemical approach”, Chem. Mater., 10, 1998, pp 3278-3281.

Lam et al have fabricated silicon nanoparticles by ball milling graphite powder and silica powder, this process being described in J. Crystal Growth 220 (4) p 466-470 (2000). Arujo-Andrade et al have fabricated silicon nanoparticles by mechanical milling of silica powder and aluminium powder, this process being described in Scripta Materialia 49 (8) p 773-778 (2003).

The silicon particles may be formed in various shapes, or display particular internal or external features depending on the particular application. These shapes and features may be formed during particle formation or post particle formation, for example using anodisation or etching as described herein. Said shapes and features include but are not limited to spheroids, cuboids, plates, cylinders, flakes, lozenges, barbs, spikes, hollow spaces, sponge-like formations, inter-connected chambers, tubes and capillaries.

Silicon microparticles or nanoparticles may be transformed into a porous agglomerated form by thermal processing, compression techniques or by the application of centrifugal forces. The agglomerated forms comprise a unitary body with macropores and/or mesopores and/or micropores.

PCT/GB2005/001910, the contents of which are incorporated herein by reference in their entirety, describes how particulate silicon, which may or may not be porous, may be consolidated to form a multiplicity of bonded silicon particles typically under the influence of pressure. The pressure may, for example be applied uniaxially or isostatically. Typical uniaxial pressures may be in the range of 10 MPa to 5000 MPa and the isostatic pressure may be in the range of 10 MPa to 5000 MPa.

The consolidation may be carried out such that the unitary body or silicon structure formed possesses a surface area greater than 100 cm²/g and preferably greater than 1 m²/g.

The consolidation of the silicon particulate product may result in a porous unitary body, the pores being formed from the spaces between the bonded silicon particles. However, the free silicon particles may themselves be porous prior to consolidation, for example by the use of stain etching or anodisation techniques. The consolidated product or so-called unitary body may itself be further porosified by anodisation or stain etching and/or may be fragmented. Fragmentation techniques include mechanical crushing or the use of ultrasonics.

The formation of the unitary body may be carried out within a selected temperature range. Cold pressing means that the consolidation is carried out up to a temperature of about 50° C. and from as low as −50° C.

The surface area of a silicon unitary body formed by a cold pressing technique may be high, relative to that of a silicon unitary body formed by a hot pressing technique. This is because hot pressing can result in rearrangement of the surface silicon atoms, causing cavities and defects to be removed.

The consolidation process may comprise combining the particulate silicon prior, and/or during and/or after consolidation with any additional materials to be loaded in such a manner that the additional material is located in the pores between the bonded silicon particles.

The tissue marker may be made using multiple manufacturing techniques. Such techniques include: packing pSi particles into gelatin capsules; use of polymer binders to formulate a pSi-binder tablet; formation of a gelatin-pSi lozenges by combining pSi with gelatin dissolved in glycerin whereby the gelatin forms an erodible matrix around pSi; extrusion and spheronisation of pSi combined with binder; embedding of pSi within a biodegradable polymer (for example, polycapralactone polyvinyl alcohols and/or poly-lactic-co-glycolic acid); and/or melt extrusion tablet formation combining pSi with biodegradable polymers (for example poly-lactic-co-glycolic acid and poly-lactic-acid). The pellet form formulated using any of these techniques may be coated to confer more desirable characteristics.

The porous unitary body may alternatively be formed by porosifying a pre-shaped unit of bulk silicon, such as a rod or tablet. Where high levels of porosity are required, the bulk unit may be porosified by anodisation such that one or more of the bulk units are linked as the anode in an electrolytic cell with the cathode formed by an encompassing inert (for example platinum) mesh in the form of a cylinder or other appropriate shape. Where lower porosity is required, the bulk unit may be porosified by stain etch such that one or more bulk units are immersed in appropriate HF etching solutions for a sufficient period of time, and where appropriate with agitation and/or circulation of the etchant solution to achieve the desired levels of porosity.

The silicon particles may be classified. Classification is defined as sorting particles into groups such that all particles within a group share the same characteristic, said characteristic being different to particles within other groups. Typical classifications characteristics include size, density, chemical composition, and other physical and chemical properties that permit sorting of particles. For example, for imaging the lymphatic system, particles of the size range 10 to 500 nm diameter are preferred, as particles smaller than this range may not accumulate in the lymph nodes and particles larger than this range may not migrate through the lymphatic system. For imaging the vascular system, particles of size range 10-8000 nm, more specifically 10-1000 nm diameter are preferred, as the internal diameter of blood capillaries is typically about 4-9 μm.

Biodegradability of the particles may be an important feature in some applications of the imaging agent. Biodegradability is related to surface area and wall thickness and as such may be partly characterised by particle density. Particle density also impacts imagability under a number of modalities, in particular CT/x-ray. Classification of particles by density in order to select particles exhibiting particular biodegradation and imagability properties may be an important feature of certain applications of the imaging agent.

Particle size and shape may also be an important aspect in the structural characteristics of pellets formed from silicon particles. The range of sizes and shapes of constituent particles in pellets formed by cold pressing, hot pressing, tablet molding or other methods described herein, may affect the pellet's fragility, degradability and biocompatibility.

Classification can be accomplished in numerous ways well known in the art. Size classification methods include sieving, filtration, laminar flow, electrophoresis and others. Density classification methods include centrifugation, flotation and other techniques.

The density of the imaging agent is an important feature. The density of silicon in the imaging agent is proportional to the imagability of the imaging agent under modalities sensitive to density such as CT and x-ray. The density of the imaging agent can be considered to be dependent upon two aspects. The first aspect being the density of the silicon in the constituent particles of the imaging agent, the second aspect being the concentration of the constituent particles within the carrier medium, whether the carrier medium is liquid as in the case of a contrast media application or solid as in the case of a tissue marker pellet or semi-solid as in the case of a gelatinous carrier medium.

The present inventors have found that a broad range of modalities give rise to particularly useful images when the following densities and/or concentrations of silicon are used. For example, a pellet or powder comprising porous silicon which has a density greater than about 0.8 g/cm³ of porous silicon is imageable under a broad range of modalities including CR, CT, MRI and ultrasound, particularly ultrasound and CT. Preferably, the porous silicon is anodised and the average mesoporosity of the pellet is greater than about 50 vol %, and for the powder the porosity is greater than about 70 vol %, for example, in both cases, about 50 vol % to about 90 vol %. Ultrasound images may be generated at densities as low as about 0.5 g/cm³. Prior to forming the pellet of density greater than about 0.8 g/cm³, the porosity of the silicon may typically be about 70 vol %.

Porous silicon, when present in liquid or gel formulations (including suspensions and the like), is preferably present in a concentration range of about 0.001 g of porous silicon per ml of total formulation up to about 2.2 g/ml. More preferred is about 0.005 g/ml to about 1.5 g/ml with 0.05 g/ml to 0.5 g/ml being even more preferred. When present in these concentration ranges, the porosity of the porous silicon is preferably about 50 to 70 vol %. The porous silicon may comprises, or consist essentially of, or consist of, a low porosity, phosphorous doped porous silicon. For example, Brachysil™ which is commercially available from pSiMedica (UK) is a high phosphorous (0.85-1.38% w/w, as measured by HF digest and Inductively Coupled Plasma Optical Emission Spectroscopy, ICP-OES) doped stain etched polycrystalline silicon of porosity 5 vol % and d₅₀ equal to 30 μm+/−3 μm. Though the overall porosity of the Brachysil™ is 5 vol %, the outer layers of the particles have significantly higher porosity than the core which is essentially non-porous. The phosphorous doped porous silicon powder samples are particularly useful in connection with x-rays, CT and MRI. The phosphorous may be ³¹P or ³²P. When ³¹P is present, the porous silicon sample may be referred to as cold.

The effective density of the imaging agent may vary over time as the body's fluids dilute the carrier medium and disperse the constituent particles. This variation may be rapid, as when a silicon contrast agent is injected into the vasculature and is rapidly diluted by blood or it may be slow, as when a silicon pellet slowly degrades and disperses within a tissue.

In order to provide a useful contrast signal under a density sensitive modality, the effective density of the silicon imaging agent should be sufficiently different from the surrounding tissues and fluids. In muscle tissue, an effective silicon density of at least 0.8 g/ml for particles of around 65% density, preferably at least 1.0 g/ml is suitable for imaging using x-ray/CT. In breast and other fatty tissues, an effective silicon density of at least 0.6 g/ml is preferred for imaging under x-ray/CT.

The imaging agent may be modified in such a way in order to render it suitable for use in molecular imaging techniques. Molecular imaging is generally defined as the measurement and imaging of biological processes in living organisms at the molecular and cellular level. Molecular imaging enables the provision of images of specific molecular pathways in the body, particularly disease targets. Advantageously, molecular imaging may allow for the detection, diagnosis and treatment at the earliest stages of disease development. For successful molecular imaging, a combination of an imaging system and a specific imaging probe is required. In selecting a suitable probe, the basic principle is to identify a specific receptor site associated with the target molecule that characterises the disease process being studied. A molecular imaging probe that binds specifically to this target molecule is then chosen. The probe may be a small molecule, such as a receptor ligand or an enzyme substrate, or a higher molecular weight affinity ligand such as a monoclonal antibody or a recombinant protein. The imaging agent is bound to the imaging probe of interest, for example a peptide or antibody or antibody fragment with high specific affinity for a particular target, by binding it to the silicon using known techniques; for example see Tinsley-Bown et al in “Tuning the pore size and Surface Chemistry of Porous Silicon for Immunoassays, Phys. Stat. Sol. A, vol. 182, pp 547-553, 2000. On administration into the body, the high specificity probe may be incorporated into the target tissue and may be imaged using one or more of the modalities suitable for use in the present invention. In this fashion, the imageability of the silicon directs the clinician to abnormal tissue as targeted by the specific probe.

The noninvasive imaging modalities utilized for molecular imaging include positron emission tomography (PET), single photon emission computed tomography (SPECT), magnetic resonance imaging (MRI), ultrasound, and computed tomography (CT). Techniques specific to small-animal imaging include bioluminescent imaging (Blm) and fluorescent imaging (Flm). Variations and subcategories of these modalities are also available, including optical coherence tomography, fluorescence or luminescence imaging, MR microscopy, photoacoustic US, and US biomicroscopy. There are also combinations of modalities that perform dual x-ray/gamma imaging, CT/PET, MR/PET, and other combinations. For example, MRI has shown promise in stem cell and lymphocyte trafficking studies and in pharmacological research for a variety of disorders. MRI has a wide array of applications to molecular imaging, including clear anatomic depiction, the study of blood flow changes in tissues with pharmacologic or other functional activation, spectroscopic quantification of metabolite concentrations, the generation of pH maps, studies of vascular volume or permeability, pharmacokinetic studies of chemotherapeutic agents, the denoting of gene expression, and the imaging of probes that are activated only when they come into contact with tissues of interest.

Molecular probes can be categorized either as constant or activatable probes. Activatable imaging agents (smart reporter probes) are molecular beacons or sensors that undergo physiochemical change and become detectable only after specific molecular interaction with the target. Thus the target specificity is high. Activatable near-infrared (NIR) fluorochromes, for example, are synthesized to detect, localize and quantify specific protease activity. These activatable imaging agents have unique quenching-dequenching properties such that they become highly fluorescent when specific peptide sequences are enzymatically cleaved by protease, with signal amplification of up to 1000-fold. Continual Emission Probes, such as radiolabeled probes (for PET and SPECT imaging) produce signal constantly through the decay and/or of the imaging agent, whereas activatable probes produce signal only when they interact with their target(s) (e.g., near-infrared fluorescent probes for optical imaging).

The molecular imaging agent described in this invention may be surface modified or may include elements in the porous silicon pores that become released or activated once the porous silicon robe has come into contact with the target molecule, causing signal amplification. The molecular imaging agent, comprising silicon, may therefore be used as an activable probe or a continual emission probe.

Furthermore, the silicon, more specifically porous silicon, molecular imaging agent can be constructed to carry genes or therapeutic agents. The porous silicon agent would typically biodegrade at a specific site of disease to deliver the contents to the targeted tissues.

A combination of one or more radio-isotopes and a specific probe may be combined with the silicon framework of the imaging agent, thus allowing the imaging agent to be localised by techniques such as PET and SPECT. The silicon and the molecular probe may be imageable using different modalities thus allowing for dual imageability using hybrid systems. For example, the porous silicon may be imageable on CT and the further incorporated radio-isotope may be visualised using PET or SPECT. In general, PET/CT, SPECT/CT, optical imaging and MRI/CT are at present the preferred modalities for molecular imaging using techniques such as those described above.

The selection of the probe is influenced by a number of factors. The probe is safe and not alter the disease process being studied, able to reach the target in sufficient concentration while not accumulating in other tissues and be retained long enough to be detected. The precise nature of the imaging agent will be, to some extent, determined according to the nature of the specific molecular target, imaging probe and the particular imaging modality or modalities being used. The specific molecular targets are related to applications including gene therapy; cell trafficking; immunotherapy; drug development; the detection, diagnosis and therapy associated with cardiovascular diseases such as atherosclerosis, thrombosis, myocardial infarction; neurological diseases such as Alzheimer's disease (AD), Parkinson's disease (PD), multiple sclerosis (MS), hyperactivity and attention deficit disorders; cancer; primary immunodeficiencies; autoimmune disease.

In monitoring of cell trafficking, the silicon molecular imaging agent can be used to look at different properties of cellular trafficking including metastasis, stem cell transplantation, and lymphocyte response to inflammation. Another method related to cell trafficking and monitoring of therapeutics in cancer research is to use antibodies and antibody fragments for imaging and radioimmunotherapy. The purpose of engineering antibodies has been to construct fragments with high affinity and ideal pharmacokinetics (rapid binding of the target tissue and clearance from the blood pool).

In contrast to cell and tissue culture, in vivo animal models using the described silicon molecular imaging agent, allow the assessment of phenomena such as tolerances, complementation, and redundancy in biological pathways. Molecular imaging permits both the temporal and the spatial biodistribution of a molecular probe and related biological processes to be determined in a more meaningful manner throughout an intact living subject.

Contrast Agents for Use in the Body. Including Circulatory and Other Systems

The silicon comprising imaging agent for use in the methods of the present invention may be suitable for use as a contrast agent for use in the body system including the circulatory system of the human or animal body. The imaging agent may be used as a contrast agent in the vasculature, the respiratory system, the lymphatic system, the muscoskeletal system, the reproductive system, the nervous system, the renal/urinary system, the alimentary system, especially the alimentary and lymphatic systems. Such a contrast agent may be referred to herein as a mobile contrast agent.

Use of the mobile contrast agents according to the present invention seeks to provide one or more of the following: high radiopacity (visible on x-ray procedures); safe and easy to administer to the human and animal body, MRI visibility (i.e. be paramagnetic), echogenicity (i.e. be visible on ultrasound); low diffusion; low blood solubility. The contrast agents for use in the present invention advantageously provide a safe toxicological profile and low allergenicity and inflammation risk when injected into the bloodstream, taken orally, inhaled or administered subcutaneously/intralymphatically. The contrast agent is preferably visible under one, or a combination of modalities including: MRI, ultrasound, x-ray, CT, optical imaging, infrared imaging, thermal imaging, gamma scintigraphy, PET scintigraphy, and derivations thereof. For the avoidance of doubt this includes the use of hybrid systems, for example PET and CT hybrid systems.

The shape and size of the contrast agent may be varied to enhance visibility under one or more modalities and/or to enhance dispersion through one or more systems. For example, for use in the respiratory system, a finely sized silicon particle in a suspension suitable for aerosolizing is preferred. The silicon comprising agents may be presented in the form of aggregates or agglomerations. In particular, this may also be the case for orally administered agents.

The size of the particles or substantially all of the particles may be in the range of from about 0.1 nm to about 1000 μm in diameter. More particularly, the preferred range is 0.5 nm to 300 μm and more preferably 1 nm to 50 μm. The size of the particles is measured using a known technique, e.g. scanning electron microscopy. Alternative techniques include, for smaller particle sizes of about 0.5 nm to 10 μm, small angle neutron scattering, laser Doppler anemometry, differential mobility analysis, centrifugal sedimentation and, for larger particle sizes of about 10 μm to about 950 μm, one or more of optical microscopy, laser diffraction, gravitational sedimentation, coulter counting, sieving.

The diameter of blood capillaries is about 7 or 8 μm and can be as low as about 4 μm. Particles smaller than this, including those smaller than 4 μm, for example 2 or 3 μm, may perfuse small vascular channels, such as the microvasculature, while at the same time providing enough space or room within the vascular channel to permit red blood cells to slide past the particles. Further, these smaller particles may be capable of travelling throughout the vasculature at about the same rate of flow as the blood and thus do not impede or substantially impede normal blood flow. Hence, for intravascular administration and in connection with, for example, imaging of the vasculature, it is preferred that the particles be no larger than about 10 μm in diameter. In certain preferred embodiments, the mean diameter of the particles may be about 5 μm or less. In further embodiments, particles having a mean diameter of 500 nm or less may be more preferred.

Particle size for particulate intravascular contrast agents, e.g. MRI agents and x-ray/CT agents, can relate to phagocytic activity, where particles are removed in a size dependent hierarchy, for example, by the lungs (largest particles), spleen, liver, and then the bone marrow (smallest particles). According to the particular application, it may be preferred that the particles are about, for example, as follows: greater than 300 nm for bowel contrast; 80 to 150 nm for liver/spleen imaging; 20 to 50 nm, or 20 to 40 nm for lymph node imaging and bone marrow imaging; and up to 5 μm for perfusion imaging and angiography.

Ultrasound microbubbles and microspheres larger than 10 μm have resonance frequencies below 1 MHz, while smaller bubbles in the order of 5 μm or less, will have resonance frequencies in the frequency range used in medical ultrasound imaging, i.e., 110 MHz. Hence, it is preferred that the particles have a maximum diameter of about 20 μm, with smaller particles being preferred. For example, the majority of particles should preferably be no larger than about 10 μm in diameter, with particles having a mean diameter of about 6 μm or less being more preferred.

The contrast agents suitable for use in the vasculature may be partially or more substantially porosified using, for example, the anodisation and/or etching techniques described above. They may also comprise derivatised porous silicon.

The porosity of the porous silicon which may or may not be derivatised may be 1 vol % to 99 vol %, preferably 20 vol % to 90 vol % and more preferably 40 vol % to 80 vol %. The porosity of the porous silicon may be about 5 vol %. The porous silicon material may have a porosity of about 5 vol % and a mean particle diameter of 30 μm and be doped with phosphorous.

The porous nature of the porous silicon may entrap air or other gases within the porous structure. The porous silicon for use in the present invention may further comprise gas entrapped within the pores. Differences in acoustic impedances of adjacent materials dictate the magnitude of the returned ultrasound echo, with greater differences leading to a stronger reflection. The significant differences in acoustic impedances of the gas, porous silicon and biological tissues give rise to a highly echogenic effect and visibility of the porous silicon under ultrasound examination.

The silicon, more preferably the porous silicon, for use in the present invention, may be in the form of a shell or bubble containing air or other gases. The air or other gases may be present in combination with an excipient or coating which serves to stabilise the air or other gases which are entrapped within the silicon pores.

The contrast agents according to the present invention may be administered using a range of techniques including intraveneously, orally, per-rectally, per-vesically, per-vaginally, endoscopically, intradermally, subdermally, intrathecally, subcutaneously, intralymphatically or via inhalation. In contrast to the methods relating to tissue markers, contrast agents for use in the vasculature need to be able to move relatively freely around the vascular systems of the human or animal subject.

In order to improve the visibility of the images generated using the present methods, further materials may be included with the contrast agent. For example, additional stable and/or unstable ions (such as radionuclides) may be associated with the contrast agent, particularly when it comprises porous silicon. These further materials may be combined with the silicon or it may be fabricated in-situ by the transmutation of silicon. Further, the contrast agent may have associated with it, stable and/or unstable ions, isotopes or molecules or combinations thereof which improve the visibility of the contrast agent to one or more modalities. These further materials may be incorporated within the pores of the silicon or within the pores formed by the agglomeration of silicon particles which may themselves be porous. These further materials may also be incorporated within the silicon matrix and/or may be covalently bonded to the silicon. The addition of radionuclides allows for visualisation with hybrid imaging techniques. For example, by using gamma or PET scintigraphy in combination with CT or MRI, concurrent imaging on hybrid SPECT/CT, PET/CT and experimental PET/MRI imaging systems may be achieved.

The contrast agent may also be modified in such a way as to make it suitable for virtual endoscopy. Virtual endoscopy (VE) is a medical imaging technique which uses CT and/or MRI images with computers to create two or three dimensional images of hollow organs such as the large bowel or airway. The principles of VE are understood by those skilled in the art and are described, for example, by Wood, B. J. and P. Razavi (2002), “Virtual endoscopy: a promising new technology.” Am Fam Physician 66(1): 107-12. Broadly, CT and/or MRI images of the structure or structures of interest are acquired and reformatted to create a volume of virtual data. This data is interrogated computationally to determine which elements of the data set are representative of anatomical details, and which are representative of extraneous material such as bowel contents or bronchial air. Digital removal of the extraneous material leaves an image of the structures of interest. Advantageously, VE provides similar information to conventional endoscopy, but without the need for potentially hazardous, uncomfortable and invasive insertion of conventional endoscopes. Successful VE requires the provision of high quality images which can be digitally enhanced using appropriate computer hardware and software. VE operates by removing information which obscures the structures of interest in the acquired images, such as contents in the colon which obscure the mucosal surface of the bowel. Digital removal of unwanted image elements requires that those elements be easily and readily identified such that accurate delineation of the structures of interest from the remainder is achieved. This process is termed image segmentation, and relies on the accurate differentiation between structures within the image. As is well known in the art, accurate image segmentation is critical to the success of VE, and many approaches have been described in the literature, for example see Tiede, U., N. von Sternberg-Gospos, et al. (2002), “Virtual endoscopy using spherical QuickTime-VR panorama views.” Stud Health Technol Inform 85: pages 523-8 and Seemann, M. D., M. Heuschmid, et al. (2003), “Virtual bronchoscopy: comparison of different surface rendering models.” Technol Cancer Res Treat 2(3): 273-9.

The imaging agent may be formulated to optimise suitability for VE applications. For colonoscopy an ingestible form of porous silicon is preferred. In this embodiment, the invention may be an imaging agent and a separate quantity of liquid, together with instructions for preparing an ingestible solution or suspension of the imaging agent in the liquid, wherein the imaging agent comprises or includes porous silicon. Alternatively the imaging agent may be provided ready made up in a solution or suspension. The invention may also be in the form of an enema administration. The invention may also be in the form of an aerosol for administration to the lungs and airways.

The imaging agent may be combined with chemical moieties which enable preferential binding to particular cells, cell types, tissues, organs or systems. Such moieties may include ligands, peptides, antibodies, antibody fragments, recombinant proteins and other molecules familiar to those skilled in the art. The imaging agent may, optionally, be combined with further chemical moieties to render it more distinguishable from normal anatomy under one or more imaging modalities.

According to the particular application, it may be that the imaging agent be formulated from particles of diameter greater than 300 nm. The imaging agent may also incorporate gas-filled pores and cavities.

The invention may be formulated to yield an image with a greater or lesser homogeneity under one or more imaging modalities. It may be that, for example, a homogeneous particle containing gas-filled pores may be preferred for ultrasound, whilst a formulation of differing particle sizes may be preferred for computed tomography.

The invention may be combined with aqueous or lipid-based solutions to aid in homogeneous dispersion through hollow body systems.

The contrast agent may be further utilised to monitor the effectiveness of therapy. Also, the contrast agent may be chemically modified. More specifically the surface of the silicon may be modified in order to attach antibodies, aptamers, oligonucleotides, proteins, sugars or lipids.

The surface, external and/or internal, of the silicon may be modified in order to enhance or retard the rate of biodegradation, resorbability, excretion or other form of metabolisation of the contrast agent from the body, in particular from the vascular system into which the agent is administered.

The surface, external and/or internal, of the silicon may be modified to enhance the dispersion, solubility, diffusion and other miscible characteristics of the contrast agent within the carrier fluid and within the bodily fluid or fluids contained in the vasculature into which the contrast agent is administered.

The surface, external and/or internal, of the silicon may be modified in order to cause the body to preferentially retain the contrast agent within one or more parts of the vasculature into which the contrast agent is administered.

The surface, external and/or internal, of the silicon may be modified in order to prevent particles of the contrast agent from lodging, adhering, depositing, precipitating, abrading or otherwise interacting with the walls, linings, membranes, tissues and organs associated with or connected to the vasculature into which the contrast agent is administered.

Typically the contrast agent suitable for use in the vasculature will be delivered in combination with a carrier system comprising a fluid such as a liquid. For example, the contrast agent, which may be in the form of microparticles and/or microbubbles, is suspended in an aqueous, preferably a saline carrier, for example including 0.9% w/w sodium chloride and is suitable for being injected into the subject. Other carrier systems include phosphate buffered saline solution, typically 10 mM and with a pH of 7.4, a HEPES buffer (eg 20 mM, pH 7.4), solutions of glucose (eg 5% w/w in water). The use of isotonic solutions is also suitable such as isotonic glucose solutions.

The contrast agents may also be suspended in a formulation comprising one or more solubilizing, stabilizing, suspending, dispersing, diluting, emulsifying, gel forming, and/or other excipients described previously in this application.

Physicochemical techniques to solubilize and/or suspend the microparticles and/or microbubbles in the contrast agents can include: silicon surface modification, pH adjustment, cosolvents (mixtures of miscible solvents), complexation (interaction between the active substance and a soluble complexing agent), micelles (surfactants self-assemble into micelles when the surfactant monomer concentration reaches the critical micelle concentration), liposomes (closed spherical vesicles composed of outer lipid bilayer membranes surrounding the active particle), emulsions (heterogeneous mixtures of water, oil, surfactant and other excipients), liquid suspensions (two-phased systems consisting of a finely divided solid dispersed in a liquid) and gels (semisolid systems consisting of suspensions made up of either small inorganic particles or large organic molecules interpenetrated by a liquid).

### Tissue Marker

According to one of the aspects, the present invention provides a method of tissue marking. The method includes the use of a detectable tissue marker, delivered to a tissue site, optionally with the use of one or more of a range of modalities, for later detection via one or more of a range of modalities. The placement of tissue markers according to the methods of the present invention may be carried out using minimal or non-invasive methods. For example, the tissue marker may be delivered into the body to a desired site by injection using a hypodermic needle and syringe, or another similar instrument, or percutaneously, with the assistance of a biopsy probe. The tissue marker may be visualised, including for guiding means, with a range of modalities. These include one or more of the following: x-rays, ultrasound, CT, MRI, mammography, optical imaging, scintigraphy (including PET scintigraphy and gamma scintigraphy), near infrared imaging, digital imaging, and further includes the use of image fusion. The types of tissues which may be marked include the colon, rectum, prostate, breast, brain, kidneys, liver, lungs, bone, oropharynx, skin, lymph nodes, spleen, adrenals, testis, ovaries, ureter, nerve, bladder, heart, and soft tissues in general including muscles.

As the tissue marker may be used in marking the skin, the methods of the present invention include the use of so-called tattoos for use, for example, in positioning patients, including repeat positioning in radiation therapy. Such tattoos may be visible to the human eye and/or under other wavelengths of light such as ultraviolet light. Typically, such a tattoo will be biodegradable or resorbable. Advantageously, the tattoo may be loaded with antibiotic to minimize the risks of infection. Loading may utilize the techniques described in WO 05042023 the contents of which are hereby incorporated by reference in their entirety.

The tissue marker may be administered in a range of forms and using a range of methods. For example, the tissue marker may be in particulate or pellet form. One or more of the size, shape and porosity of the particles or pellets are readily varied in order to enhance retention in the target tissue by controlling the rate of biodegradability, and/or enhance visibility under one or more modalities. Methods of administration include injection, implantation and imbedding. Advantageously, the methods provided by the present invention do not require the use of complex and additional tools during implantation. When the tissue marker is in the form of a pellet, it may comprise external features to assist in anchoring the pellet into the surrounding target tissue or to assist in the imaging of the tissue marker under one or more modalities.

The silicon particles can preferably be of an average size in the range from about 10 nm to 200 μm, more preferably 5 μm to 100 μm.

An advantage of the tissue markers used in the methods of the present invention is that they may be designed and engineered to suit individual medical needs. Once delivered to the anatomical site, the tissue marker should stay in position over an appropriate period of time. The degree of biodegradability or resorbability of the tissue marker may be tailored by varying the size of the particles or pellets of the silicon and/or its porosity, and/or the excipient composition, if present, such that the agent remains visible under the appropriate modality or modalities over the required period of time. Examples include complete biodegradation within 29 days, or 6 months or 1 year. For example, the porosity of the silicon determines its half life in the body, thus enabling it to biodegrade after a suitable period of time leaving little or no trace of the tissue marker in the tissue so that further surgical procedures are not required in order to remove it. A further example of tailoring the present invention is the incorporation of excipients described above such as disintegrants and alkalizing agents which increase the rate of degradation of the tissue marker, and/or coatings around the tissue marker to prevent contact of the pellet and water to inhibit degradation, allowing imageability for predetermined periods.

The methods of the present invention allow for particularly accurate marking of a site, such as a biopsy site. Because the size of the tissue marker may be readily controlled, this allows for a range of particle sizes to be administered, for example, via injection, and the outline of a site such as a tumour may be accurately marked out or the cavity of a biopsy site filled. This is in contrast to more traditional methods of tissue marking, such as those involving the use of a metallic clip to mark a biopsy site, which do not necessarily provide the medical practitioner with an accurate indication of the size of the area that requires irradiation or monitoring. Some tissue locations such as the colon do not lend themselves to the use of marking clips, yet it is possible to deliver microparticles to these locations for effective marking. The tissue marker may be placed into a biopsy site after a specimen has been collected.

The tissue marker may be included in a formulation along with one or more of a pharmaceutically acceptable carrier, excipient or diluent. The formulation may comprise microparticles other than silicon. Preferably the carrier is an aqueous carrier.

The tissue marker comprises, or consists of, or consists essentially of, silicon, preferably porous silicon, and may be sized and shaped in such a way as to render it distinguishable from anatomical structures. In one embodiment, the marker has a major dimension between about 0.1 and 5 cm and more particularly between about 1 mm and 3 cm. The thickness of the pellet may vary and may be less than about 5 cm in thickness, preferably less than 3 cm, even more preferably less than 1 cm in thickness, and even more preferably all dimensions are less than 0.5 cm. The shape of the tissue marker may vary depending on the desired application and may include shapes such as spheres, irregular shapes, discs, cylinders, rods, strips, barbs, lozenges and the like.

The markers of the present invention may be implanted in a variety of conventional manners. In one embodiment, the marker may be implanted as part of a non-invasive medical procedure. For example, the marker may be implanted during a non-invasive tissue removal procedure or a biopsy procedure. The shape of the tissue marker may facilitate injection through a needle, such as a 12 gauge needle. In another embodiment, a biopsy system may be fitted with a device for implanting the marker. In a further embodiment, the marker may be implanted using a suitable needle. Alternatively, the marker may be implanted via conventional open surgical methods. Furthermore, during implantation, the marker of the present invention may be guided to a desired anatomical site by utilizing one or more imaging modalities in which the marker is detectable. Suitable modalities for guiding implantation of the marker include ultrasonic imaging, fluoroscopy, optical imaging, thermal imaging, CT, MRI, x-ray, or any other suitable imaging technique.

In embodiments of the present invention, the tissue marker may be part of the biopsy delivering system such that the biopsy apparatus, after removing the tissue of interest, deposits a pellet of porous silicon tissue marker of between about 1 mm to 1 cm in width and about 1 mm to 3 cm in length of, for example, a circular, spherical, rod-like or oval shape, optionally including external machined fasteners. The biopsy apparatus may hold up to about ten porous silicon tissue marker pellets which will allow deposition of up to about ten markers at the site of interest in the tissues. The biopsy device may hold several biopsy samples in sequential order. The porous silicon tissue marker pellet may also be deposited separately through a standard biopsy needle apparatus where, after removal of the internal biopsy needle, a separate trochar with the porous silicon marker pellet at its end is manoeuvred through the introduction needle to the site of interest. The pellet may be deposited into the tissue and both the introducing needle and inserted trochar are then removed from the biopsy/marker site or the trochar is removed to allow positioning of a further porous silicon tissue marker pellet adjacent to the original. Several pellets can thus be inserted using this technique.

The markers of the present invention may be suitable for use in a variety of procedures or treatments that involve imaging or visualisation of a particular anatomical site. The markers may be particularly useful in the field of oncology for treating lesions or other abnormal tissue sites. The term treating may include monitoring an anatomical site, staging and planning for medical procedures, performing medical procedures (e.g., radiation therapy, surgery, biopsy, drug therapy, RF ablation, and radiotherapy), and evaluating the success of a particular treatment.

The tissue marker is particularly useful in soft tissue, for example as a breast biopsy marker, including the use of Fine Needle Aspiration Biopsies (FNAB). If the specimen is found to be cancerous, the tissue marker will assist in locating the cancer for treatment such as radiotherapy and possible surgical removal. In the event the initial biopsy transpires to be inclusive another biopsy of the same site but in a different area can then be taken with the tissue marker assisting in the localisation of the biopsy area. If the specimen turns out to be benign then the tissue marker will eventually biodegrade so as not to interfere with future tissue marker placements or imaging. Location of the tissue marker may be undertaken using standard mammography techniques or other scanning techniques.

The tissue marker is also of use in precisely locating internal organs and tissues for treatment. Many organs inside the body exhibit a degree of mobility and are rarely present precisely in the same position. This can significantly decrease the accuracy of radiotherapy, leading to increased amounts of irradiation and consequential damage to surrounding normal tissue. The tissue markers when used as markers for internal tissues or organs assist in the localisation accuracy of radiotherapy and image-guided surgery.

There are numerous advantages associated with more precise tumour localisation. These include: the freedom to apply higher doses of radiation to the tumour, as there are less side effects; accuracy and ease of daily patient positioning; real time targeting of tumours; the ability to plan procedures and protocols of treatment on-line; image fusion, i.e. the ability to compare the precise same area of interest on different scans and different imaging modalities.

The use of precise internal location tissue markers is particularly suited to the prostate which is a movable organ. When a cancerous prostate tumour is detected, radiation is often a prime treatment modality. The side effects of this treatment can be highly unsatisfactory as damage to surrounding tissues can and often leads to permanent impotency. By, for example, inserting the tissue marker under ultrasound guidance, precise localisation of the tumour may be achieved. During treatment, an Electronically Portal Imaging Device (EPID) may then be used to obtain on-line images localising the tissue marker and this information may then be used to deliver accurate radiation doses to the cancerous tissue.

The use of tissue markers according to the present invention is also beneficial for tumour surveillance. Early diagnosis of tumours is often linked to the detection of small tumours, resulting in smaller target areas for potential radiotherapy and for tumour surveillance. Also, tumour and adjacent tissue shrinkage can be significant resulting in tissue distortion and difficulties in detecting the tumour following treatment. By inserting the tissue marker prior to treatment, accurate localisation of the treated area may be obtained allowing for more precise follow up treatment and/or assessment and monitoring of the area. The methods according to the present invention may therefore be used to assist in the visualisation and surveillance of potential tumours.

More specifically, the tissue marker may be used to monitor a site such as a biopsy site. For example, the rate of degradation of the tissue marker may be measured in order to monitor, for example, the appearance or disappearance of a tumour. The presence of a tumour, and changes in its condition, will affect the physiology, e.g. the pH, of the surrounding area which will itself result in the rate of degradation or resorption of the tissue marker being affected. The marker may be used to monitor other physiological changes such as temperature rises or pH changes in case of tumour reoccurrence. During tumour formation (tumorgenesis) there is an associated decrease in pH, for example see Gerweck, L. & Seetharaman, K. “Cellular pH gradient in tumour versus normal tissue: potential exploitation for the treatment of cancer” in Cancer Research, 56 (6), pages 1194-8. This decrease in pH may slow the rate of biodegradation of the silicon. This change in the rate of biodegradation may therefore be indicative of tumorgenesis. Anderson et al in Phys. stat. sol. (a) 197, No 2, pages 331-335 (2003) describe how porous silicon shows increased dissolution with time at alkaline pH. Leong et al in Extended Abstracts of the 5th International Conference on Porous Semiconductor Science and Technology 12-17 Mar. 2006 ISBN 84-608-0422-4 Abstract O11-05, p 141-142 which describes enhanced erosion of porous silicon material due to an increase in pH.

The methods of the present invention are suitable for use in marking bones in order to facilitate image-guided bone surgery, radiotherapy and implant studies including in the field of dentistry. For example the methods according to the present invention may be used to enhance the accuracy of CT scans when preparing fixed denture prostheses.

Advantageously, the tissue marker according to the present invention possesses one or more of a range of properties. These include its biocompatibility and safe toxicological profile plus an associated low rejection and inflammation risk. It is visible via a range of modalities of imaging including one or a combination of, for example, MRI, CT, ultrasound, mammography, digital imaging, optical imaging, thermal imaging, fluorescence imaging, PET scintigraphy, infrared imaging, gamma scintigraphy and x-ray. The biodegradability and size of the tissue marker are variable and controllable to suit particular patients. The tissue markers of the present invention are readily incorporated into formulations and suitable for standard techniques of delivery.

Optionally, the tissue marker may include additional materials to enhance the imaging characteristics of the marker and/or the multi-modality imaging characteristics of the marker. In order to enhance the x-ray opacity of the marker, one or more metals may be added to the tissue marker. The one or more metals could be incorporated via a range of techniques including electroless plating, electroplating, co-compression or co-milling. Suitable metals include one or more of the following: titanium, gold, tantalum, iridium, platinum, tungsten, rhodium, palladium, silver, molybdenum, copper, iron, gadolinium, manganese, chromium, zinc, titanium, barium, magnesium, calcium. Other suitable materials include stainless steel. Other additional materials include one or more of the following: radionuclide, therapeutic drug, healing promotant, radiopharmaceutical, anti-infective, or other beneficial substance for timed, slow or triggered release which may be controlled by varying the biodegradability of the tissue marker, for example by varying the porosity of porous silicon.

### BRIEF DESCRIPTION OF THE DRAWINGS

Embodiments of the invention will now be described, by way of example only and without limitation, with reference to the accompanying drawings and with reference to the following non-limiting examples, in which:

FIG. 1 is a CT image (axial) of suspensions of stain etched porous silicon in a sample of bovine muscle tissue described in Example 5 (divisions on ruler equivalent to 10 mm);

FIGS. 2a and 2b are thermal images according to Example 6a illustrating before and after administration of a suspension of a porous poly-Si/NaCMC sample to a chicken breast tissue;

FIGS. 2c and 2d are thermal images according to Example 6b illustrating before and after administration of a suspension of a porous poly-Si/NaCMC sample to a chicken breast tissue;

FIGS. 3a and 3b illustrate (a) warming of a cooled porous silicon pellet and an equivalent piece of chicken breast tissue—Example 6c, plus (b) cooling of a warmed porous silicon pellet and an equivalent piece of chicken breast—Example 6d;

FIG. 4 illustrates the use of porous silicon compared to an ink as a tattoo and is described in more detail in Example 10;

FIGS. 5a-d relate to ultrasound images comprising porous silicon compared with a commercially available contrast agent;

FIGS. 6a-c are images generated using an inverted fluorescent microscope of porous silicon particles suspended in PBS and labelled with either Integrin α-7 (A), M-cadherin (B) or Pan-Laminin (C) primary antibodies and fused with C2C12 cells.

### EXAMPLES

### Example 1

An electronic grade single crystal silicon wafer of purity 99.99999%, 5-15 mΩ cm resistivity and 150 mm diameter is anodised at 30 mA/cm² for 90 minutes. A much higher current density is then applied for a few seconds to create an underlying thin very high porosity layer that will facilitate removal of the thick porous silicon layer from the non-porous part of the wafer. Upon immersion of the anodised wafer in an alcohol rinse bath, the fully intact membrane is released. This generates a 67-75 vol % porosity, 145 μm thick mesoporous membrane. The membrane is then crushed into mm size granules, and jet milled and classified. This generates a porous silicon particulate product comprising electronic grade silicon of purity 99.999% having a broad distribution of particle size between 25 and 125 μm diameter. It is then treated in aqueous HF solution for ten minutes, washed in deionised water for ten minutes, before being air dried on filter paper for ten minutes, in order to remove surface oxide prior to compression. 100 mg of the resulting dried powder is then transferred to a 1-5 mm diameter die and compressed uniaxially under vacuum at 1000 MPa. The resulting pellet has a macroporosity in the range 20-50 vol % and a mesoporosity in the range 50-75 vol %. The pellet is sterilised by gamma irradiation and is in a form suitable for administering via injection into the target tissue of a patient by a biopsy needle. The marked tissue is suitable for imaging.

### Example 2

A microparticle contrast agent suitable for use in the present invention is prepared as follows. Electronic grade polycrystalline silicon powder of purity 99.999% is jet milled and classified into a tight size distribution with a mean diameter of 1 μm, a d₅₀ of 3 μm and a d₉₀ of 5 μm. 50 g of the classified powder is subjected to a 36% HCl acid wash and water rinse. The dried batch is then stain etched to a porosity in the range 40-80 vol % using HF/Nitric acid solutions under temperature regulation. On completion, the reaction is quenched by addition of cold water, the slurry stirred for 2 minutes and the product isolated by filtration. Water rinsing is followed by acetone and ethanol rinsing. The porous particles are suspended in a suitable formulation that after sterilisation is ready for intravenous injection.

### Example 3

A range of samples were prepared for testing with ultrasound. These were as follows:

(a) Bulk silicon powder (Metallurgical Grade—MGSi);
(b) A powder comprising silicon and iron (FeSi);
(c) A multi-layer porous silicon powder (MpSi) produced from a membrane (200 repeats) with the powder being hand-milled from 5 to 10 minutes;
(d) High porosity porous silicon powder (HpSi). This sample was produced by anodising a p+ wafer in a standard electrolyte. The powder was produced when the porous silicon self-detached itself from the wafer during the drying process. It was milled by hand and the porosity was estimated to be greater than 85%.

Ultrasound measurements were taken using an ESAOTE MEGAS ultrasound machine using a standard linear probe at a frequency of 7.5 MHz, and at a constant depth of 5 cm (2.5 cm focal point) and a power of 75%.

Each of the samples was injected in the form of a suspension into a poultry muscle. These were prepared by adding each of the silicon samples to 1 ml of saline and 3 mls of saline. The results indicated that each of the samples could be readily visualised with ultrasound. The degree of echogenicity with distal acoustic shadowing was highest for samples (b) and (c), followed by sample (a) and then sample (d).

### Example 4

A range of pSi pellets were prepared for testing under the imaging modalities of x-ray, ultrasound, CT and MRI.

The pellets were generated from powdered porous silicon material hand milled from a pSi membrane which was subsequently cold pressed under various forces and pressures to produce a standard circular pellet ranging in density from 0.788-1.099 g/cm³ with an average mesoporosity of 69.2 vol %. The pellets were then inserted into tissue samples and imaged in-vitro.

Ultrasound imaging was performed using a General Electric Logiq 700 Diagnostic Ultrasound machine using a linear array transducer at 7.5 MHz with imaging set at close focus. Radiography examinations were performed on a Siemens Maximus M80 with an Amplimat 5 radiography machine, linked to an Agfa Computerized Radiography (CR) system. All radiography imaging was performed at exposures of 40 kV and 8 mAs, with fine focus, no grid, and focus-to-film distance of 100 cm. The Computerised Radiography system plate speed was set to detail with exposure classification 100. CT imaging was performed on a General Electric Lightspeed VCT Multi (64) Slice scanner which acquired data in slices of 0.625 mm thickness and was reformatted as 1.25 mm axial scans. Scans were performed at 120 kV and 120 mA and presented with “soft tissue” window of 350 HU widths, 40 HU level. MRI was conducted on a Siemens Sonata scanner, 1.5 Tesla units. MRI T1WI and T2WI sequences were acquired. T1 images with TRs of 300-650 msec, TEs of 15 msec and T2 images with TRs of 3,000 msec and above, TEs of 100 msec and above were acquired. The images were optimised for grey scale display depending on the range of intensities read.

Results indicated that the porous silicon pellets were radiographically obvious on x-ray and CT scans, clearly echogenic on ultrasound and they created visible signal voids (or negative defects) on MRI. Results also indicated that preferably pSi pellets need to be of a density greater than 0.8-1 g/cm³ to provide clinically adequate contrast in soft tissue. Under ultrasound, all the pellets were visualised as dense echogenic surfaces causing considerable acoustic reflection and were therefore very conspicuous. Almost all of the pellets were identifiable as short, sharp, linear interfaces. With x-ray exposure, all pellets were discernable against the surrounding tissue. CT allows a greater discrimination of the density of the pellets. Density on CT imaging is measured in the form of Hounsfield (HU). In the CT scans, a solid iron pellet was used for control purposes and shows up as extremely dense (3,000+HU). Solid silicon pellets showed up as very dense at 1,000+HU, which is similar to what would be expected from very dense bone or light metal. Porous silicon, in the density range of 0.788-0.901 g/cm³, returned densities between 60 and 150 HU, thus placing it approximately in the soft tissue density range.

Porous silicon pellets of intermediate density (0.9-1.0 g/cm³) returned densities around the 200-250 HU range. This was just above the threshold of conspicuity when their densities were compared with that of the surrounding soft tissue. Porous silicon pellets of a density range 1.06-1.099 g/cm³ were slightly more visible and returned densities of around 300 HU.

When subjected to T1 and T2 weighted MRI, all pellets returned negative signal and therefore appeared as dark signal foci within the tissue.

### Example 5

A range of pSi suspensions were prepared for testing under the imaging modalities of x-ray, ultrasound and CT.

The suspensions were generated from powdered silicon material porosified using a stain-etched technique. The porosity of the material was approximately 5%, with a mean particle diameter of 30 μm. The pSi material was formulated using a 0.5% w/v solution of carboxymethylcellulose, sodium salt (NaCMC). Concentrations of 1 gml⁻¹ and 1.5 gml⁻¹ pSi in NaCMC solutions were prepared. A further 1 gml⁻¹ pSi formulation was formed with chlorhexidine gluconate and methyl hydroxybenzoate gel (CGHM). Control solutions of 0.5% w/v NaCMC and Omnipaque—240 mgl/ml as lohexol 10.36 g/20 ml injection equivalent to 4.8 g Iodine/20 ml injection were also prepared. The preparations were imaged both external and internal to a sample of bovine muscle tissue under the imaging modalities described below.

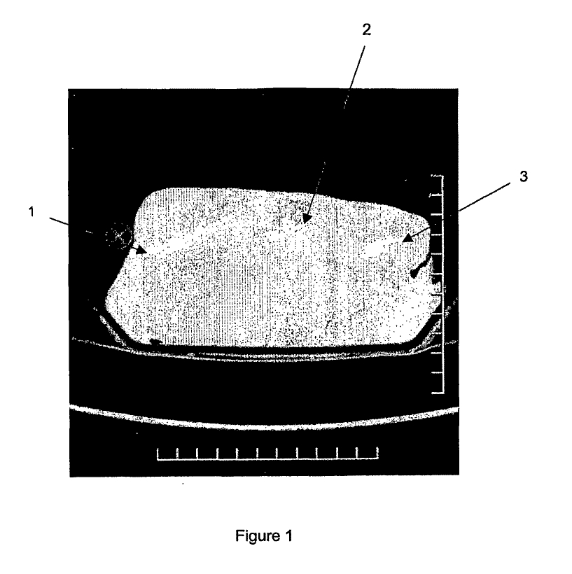

Radiography examinations were performed using Philips Optimus and Super 50 CP radiography units, which was linked to an Agfa Computerized Radiography (CR) system using a CRMD4.1 FLFS computed radiography plate. Radiographic imaging was performed at exposures of 50, 55 and 75 kV and 5 and 25 mAs, with fine focus, no grid, and focus-to-film distance of 100 cm. The Computerised Radiography system plate speed was set to detail with exposure classification 100. Ultrasonographic examinations were performed using a Toshiba Aplio using a 604 4-11 MHz linear array probe. Computed Tomography was performed using a Philip Brilliance 64 slice CT scanner which acquired data in slices of 0.625 mm thickness and was reformatted as 1.00 mm axial scans. Scans were performed at 120 kV and 50 mAs and presented with “soft tissue” window of 350 HU widths, 40 HU level. FIG. 1 is the CT image (axial), wherein area (1) corresponds to CGMH+1 gml⁻¹ pSi, area (2) corresponds to NaCMC+1 gml⁻¹ pSi and area (3) corresponds to NaCMC+1.5 gml⁻¹ pSi.

The results indicated that the porous silicon suspensions were radiographically obvious on x-ray and CT scans, and clearly echogenic on ultrasound. Contrast enhancement is greater at 1.5 gml⁻¹ pSi than 1.0 gml⁻¹ pSi. Both concentrations produce clinically adequate contrast enhancement in soft tissue.

With x-ray exposure, all solutions containing porous silicon were discernable against the surrounding tissue whilst control solutions of CGMH and NaCMC are not apparent. Under ultrasound, pSi suspensions were visualised as dense echogenic areas causing considerable acoustic reflection. Contrast enhancement was increased as the concentration of pSi in the suspension was increased. CT allowed a greater discrimination of the density of the injected pSi suspensions compared to the use of the other modalities.

### Example 6

Samples of pSi pellets and particulate suspensions comprising pSi were subjected to a range of temperatures and subsequently subjected to in-vitro soft tissue imaging under a thermal camera to observe thermal differences and thermal relaxation times as normalized with the surrounding environmental temperature. All of the powders were sourced from pSiMedica in Malvern, UK. The results confirmed that porous silicon, in both pellet and particulate form, can be visualized under the modality of thermal imaging.

Stain-etched, jet milled, high P-doped “cold” poly-silicon powder (“Brachysil™”), 30 μm sized particles, were obtained from High Force via pSiMedica (batch number CT8009R11C). Porous Si pellets were prepared by cold compression (20kN) of pSi powder which had been hand-milled and sieved to below 54 μm (average porosity 65.2 vol %) and blended for one hour with pSi powder which had been hand-milled for 15 mins and possessed an average mesoporosity of 62.1 vol %. Direct contact with the pSi powders and excipients was avoided.

Carboxymethylcellulose sodium salt, medium viscosity, was obtained from Fluka Biochemika, Item no 21902. Chicken breast tissue of approximate dimensions 15 cm length, 7 cm width and 3-4 cm thickness was used. Water for making up the formulations, suitable for injection, was obtained from Pharmacia & Upjohn, BP 100 ml, pH 5-7. Mixing was carried out using a Vortex mechanical mixer. Thermal imaging was conducted with an Inframetrics (FLIR) SC 1000 infrared X90 series thermacam linked to a computerized imaging system.

All powders (and pellets) were opened and left to stand in a fume-hood for 5-10 minutes. The carboxymethylcellulose sodium salt (NaCMC) was made up into a 0.5% w/v formulation by weighing out 0.05 g of the powder and mixing it with 10 mls of sterile water suitable for injecting. 15 g of the 30 μm stain-etched poly-Si powder was weighed out and placed into a glass beaker. 10 ml of the NaCMC solution was then added to the silicon sample, and physical mixing of the gel and pSi powder was achieved using a spatula and a vortex mechanical mixer, until a homogeneous 1.5 g pSi/ml NaCMC suspension was obtained. 2 ml aliquots of the 1.5 g/ml pSi/NaCMC suspension were then drawn up into 3 ml syringes and capped with a 16 g, 1.5 inch needle for later use. The room temperature was held at a constant 21° C. during the trials.

### Example 6a

### Heating

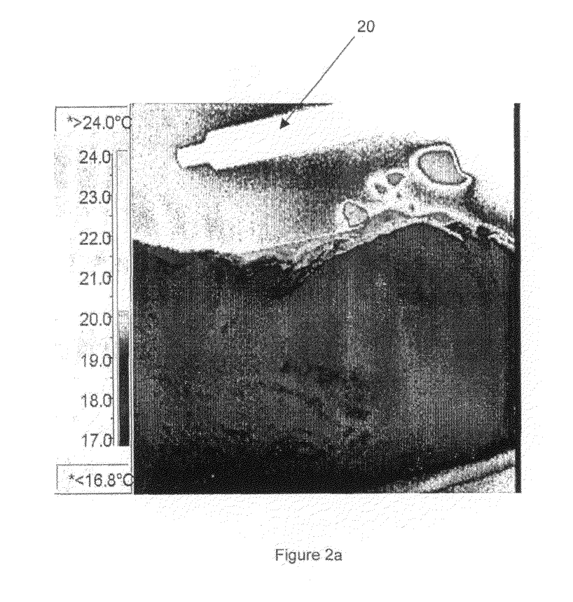

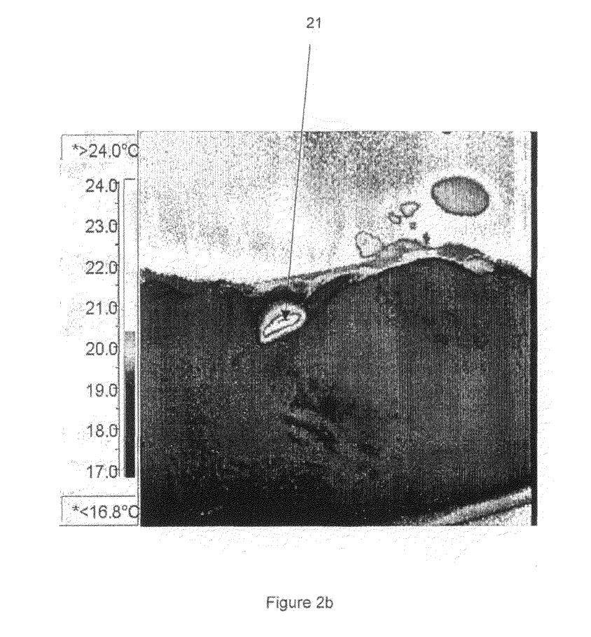

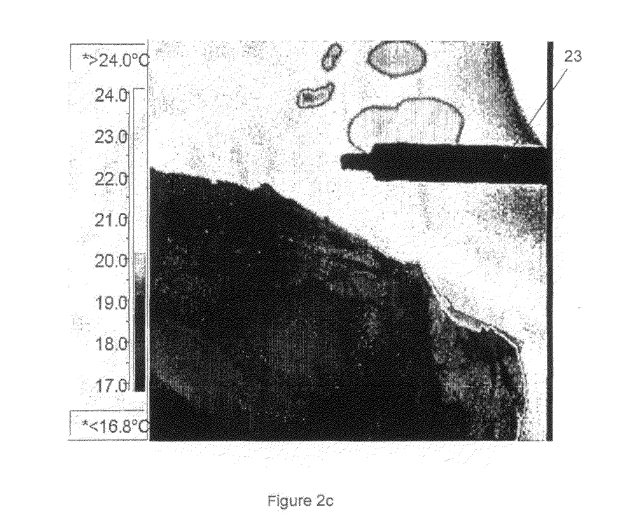

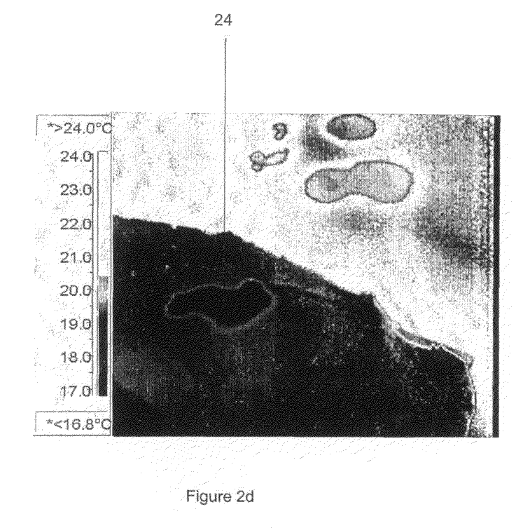

2 ml of the poly-Si/NaCMC solution, and a porous silicon pellet of 5.07 mm diameter, 1.89 mm height, 0.038 cm³ volume, 0.038 g weight and 0.996 g/cm³ density were placed into a portable 12 volt warmer set at a temperature of 40° C., until thermal equilibration occurred. Cod liver oil lubrication was used during the manufacture of this pellet. A superficial tissue incision was made into the left side of the chicken breast using a scalpel blade. The incision was approximately 5 mm deep and extended into the chicken breast a distance of about 2 cm. An initial thermal image was taken of the chicken to obtain a reference image and a survey thermal image of the pellet within the warmer was also taken to ensure the pellet temperature had equilibrated with the temperature of the warmer. The pellet was then removed from the warmer using a pair of warmed forceps. A thermal image of the pellet was taken next to the tissue sample, and the pellet was then inserted into the tissue incision. Thermal imaging of the tissue sample was then performed to record the thermal relaxation & equilibration time of the pSi pellet. A survey thermal image of the pSi suspension in the warmer was then taken to ensure the suspension temperature had equilibrated with the temperature of the warmer. The pSi suspension was then removed from the warmer and was again imaged prior to insertion into the tissue sample. Initially, an attempt was made to inject the pSi suspension into the tissue sample through a 16 G needle. However, some difficulty was experienced with this technique and so the needle was removed and 1 ml of the suspension was inserted into a superficial tissue incision made into the right side of the chicken breast using a scalpel blade. The incision was approximately 5 mm deep and extended into the chicken breast a distance of about 2 cm. Thermal imaging of the tissue sample was then performed to record the thermal relaxation & equilibration time of the pSi suspension. FIGS. 2a and 2b illustrate the thermal images obtained before and after the suspension was administered. In FIG. 2a, a syringe loaded with suspension (20) is clearly visible and in FIG. 2b the suspension has been administered at the target and is clearly visible (21).

### Example 6b

### Cooling

2 ml of the poly-Si/NaCMC solution, and a porous silicon pellet of 5.08 mm diameter, 1.89 mm height, 0.038 cm³ volume, 0.038 g weight and 0.992 g/cm³ density were placed into a medical refrigerator set at a temperature of 4° C., until thermal equilibration occurred. Cod liver oil lubrication was used during the manufacture of this pellet. The same trial protocol as listed above for Example 6a was then followed to measure the thermal relaxation and equilibration time of the pSi pellets and powder from a cold environment. FIGS. 2c and 2d illustrate the thermal images obtained before and after the suspension was administered. In FIG. 2c, a syringe loaded with suspension (23) is clearly visible and in FIG. 2d the suspension has been administered at the target and is clearly visible (24).

### Example 6c

### Cooling

2 ml of the poly-Si/NaCMC solution, and a porous silicon pellet of 5.06 mm diameter, 1.38 mm height, 0.027 cm³ volume, 0.030 g weight and 1.061 g/cm³ density were placed into a medical refrigerator set at a temperature of 4° C., until thermal equilibration occurred. Cod liver oil lubrication was used during the manufacture of this pellet. Two chicken breast tissue samples were also placed in the fridge, one sample being of similar dimensions to the pSi solution (i.e. 1 cm³) and the other of similar dimensions to the pSi pellet (i.e. 5 mm diameter×2 mm). The samples were then removed from the refrigerator and stood at room temperature. All samples were imaged using the thermal camera to observe the thermal relaxation and equilibration time of the pSi pellet and powder in comparison to the tissue samples. Initial imaging was continuous for the first 10 minutes and then became periodic every 2-3 minutes.

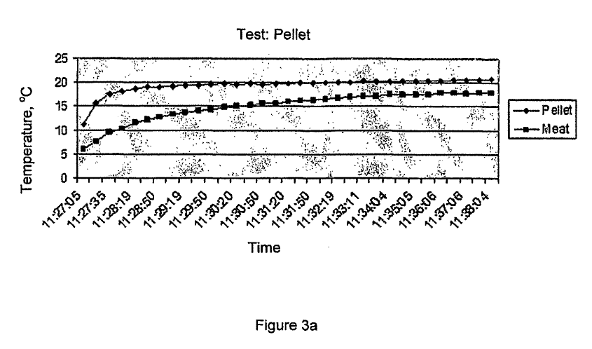

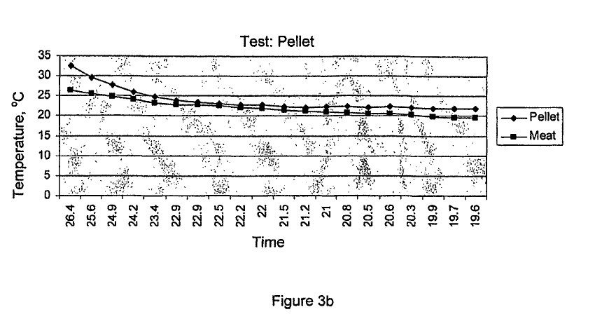

Both chicken breast samples cooled to a starting temperature of 3.1 and 3.4° C. The pSi pellets equilibrated to a temperature of 4.2° C., and the suspension equilibrated to a temperature of 5.1° C. in the medical refrigerator. Both pSi forms were visualized on thermal imaging but initially were not visually different to the tissue sample on thermal imaging. When the samples were left to equilibrate to room temperature the pellet warmed more rapidly than the tissue sample, and achieved thermal equilibration by three minutes and 16 seconds (FIG. 3a).

### Example 6d

### Heating

2 ml of the poly-Si/NaCMC solution, and a porous silicon pellet of 5.08 mm diameter, 1.89 mm height, 0.038 cm³ volume, 0.038 g weight and 0.99 g/cm³ density were placed into a portable 12 volt warmer set at a temperature of 40° C., until thermal equilibration occurred. Two chicken breast tissue samples were also placed in the warmer, one sample being of similar dimensions to the pSi solution (i.e. 1 cm³) and the other of similar dimensions to the pSi pellet (i.e. 5 mm diameter×2 mm). The samples were then removed from the warmer and stood at room temperature. All samples were imaged using the thermal camera to observe the thermal relaxation and equilibration time of the pSi pellet and powder in comparison to the tissue samples. Initial imaging was continuous for the first 10 minutes and then became periodic every 2-3 minutes thereafter.

Chicken breast samples equilibrated to a starting temperature of 26.6-27.6° C. The pSi pellet equilibrated to a temperature of 33.2° C., and the suspension equilibrated to a temperature of 37.3° C. in the portable warmer. At commencement of imaging the pellet was at 32.6° C., and the meat was at 26.4° C. The pellet cooled rapidly before reaching a similar temperature as the meat sample at 3 minutes 50 seconds, after which both cooled at approximately equal rates (FIG. 3b).

The results from the thermal imaging studies indicated that the anodized pSi pellets have a much quicker thermal equilibration time than the particulate poly-Si.

### Example 7

Example 7 illustrates the in vitro visibility of porous silicon in multiple organ systems in cadaveric tissue (adult female entire greyhound cadaver, non-fixed). Pelletized and particulate pSi suspensions were inserted into different areas of an entire canine cadaver, and the cadaver subjected to imaging under the modalities of computed tomography (CT) and magnetic resonance imaging (MRI).

All powders were sourced from pSiMedica in Malvern, UK. More specifically, the silicon samples used in this trial were stain-etched, jet milled, non P-doped “cold” poly-silicon powder (“Brachysil™”), 30 μm sized particles, supplied by High Force via pSimedica (Batch number CT7842R9C). Anodised pSi pellets were prepared by the cold compression of pSi powder which had been hand-milled and sieved to below 54 μm (average porosity 65.2 vol %) and blended with pSi powder for 1 hour which had itself been hand-milled for 15 mins (average porosity 62.1 vol %). Carboxymethylcellulose sodium salt for use in formulations (medium viscosity) was obtained from Fluka Biochemika, Item no 21902. Water for injection BP 100 ml (pH 5-7) was obtained from Pharmacia & Upjohn.

All powders (and pellets) were opened and left to stand in a fume-hood for 5-10 minutes. The carboxymethylcellulose sodium salt (NaCMC) was made up into a 0.5% w/v formulation by weighing out 0.05 g of the powder and mixing it with 10 mls of sterile water for injection. 10 ml of the NaCMC solution was added to 15 g of the 30 μm stain-etched poly-Si powder and mixed using a spatula and a vortex mechanical mixer until a homogeneous 1.5 g pSi/ml NaCMC suspension was achieved. In some of the experiments, the 1.5 g pSi/ml NaCMC suspension proved quite difficult to inject.

CT was performed with a Toshiba Asteion 4 slice CT scanner at 120 kVp, 30-50 mAs with 2 mm acquisitions reformatted as 1.2 mm scans and sagittal multi-planar reconstructions (MPRs). MRI was conducted on a Siemens Sonata scanner, 1.5 Tesla unit. MRI T1 weighted images (T1WI) and T2 weighted images (T2WI) sequences were acquired. T1 images with repetition times (TRs) of 300-650 msec, echo times (TEs) of 15 msec. T2 images with TRs of 3,000 msec and above, TEs of 100 msec and above. The images were optimized for grey scale display, depending on the range of intensities read.

### Vascular System

### Right Jugular Vein

The right jugular vein was identified and the overlying skin was shaved devoid of hair. An incision was made through the skin and the jugular vein was isolated via blunt dissection. A stab incision was made so as to penetrate into the lumen of the jugular vein and all blood and blood clots were evacuated. A porous silicon pellet of 5.08 mm diameter, height 1.67 mm, volume 0.034 cm³, weight 0.04 g and density 1.18 g/cm³ was inserted through this incision, which was subsequently sutured closed. The surgical site was closed routinely.

### Left Jugular Vein

The left jugular vein was identified and the overlying skin was shaved devoid of hair. An incision was made through the skin and the jugular vein was isolated via blunt dissection. A 1.5 cm section of the vein was ligated at either end to create a “closed” section of vasculature. All blood and blood clots were evacuated via a needle and syringe. 1.5 mls of the pSi/NaCMC suspension was injected into the area described above. The surgical site was closed routinely.

### Respiratory System

### Right Lung Field

The skin overlying ribs 7-10 was shaved devoid of hair. An intercostal thoracotomy was performed between ribs 8-9, thereby allowing access to the right caudal lung lobe. An incision was made into the lung parenchyma into which a porous silicon pellet of 5.05 mm diameter, height 1.86 mm, volume 0.037 cm³, weight 0.04 g and density 1.07 g/cm³ was inserted. The lung incision was sutured closed and the thoracotomy site was closed routinely.

### Left Lung Field

A percutaneous injection into the left lung parenchyma was attempted through intercostal space 8, using a 16 g, 3 inch needle. 2 mls of pSi/NaCMC suspension was injected via this method.

### Lymphatic System

### Right Submandibular Lymph Node (SMLN)

The right SMLN was identified and the overlying skin was shaved devoid of hair. An incision was made through the skin and the SMLN was isolated via blunt dissection. A stab incision was made into the lymph node, and a porous silicon pellet of 5.08 mm diameter, height 1.81 mm, volume 0.037 cm³, weight 0.04 g and density 1.12 g/cm³ was inserted into the middle of the glandular tissue. The stab incision was sutured closed and the surgical site was closed routinely.

### Left Submandibular Lymph Node

The left SMLN was identified and the overlying skin was shaved devoid of hair. An incision was made through the skin and the SMLN was isolated via blunt dissection. 1 ml of pSi/NaCMC suspension was then injected into the LN, and the incision site closed routinely.

### Alimentary System

### Oesophagous

The oesophagous was approached and isolated via a lateral incision and blunt dissection on the right side of the neck. A 2.0 cm section of the oesophagous was ligated at either end to create a “closed” section of lumen. All material was evacuated via a needle and syringe. 1.5 mls of the pSi/NaCMC suspension was injected into the area described above. The surgical site was closed routinely.

### Rectum

The caudal rectum & anus were evacuated of faeces and a porous silicon pellet of 5.05 mm diameter, height 1.83 mm, volume 0.037 cm³, weight 0.04 g and density 1.06 g/cm³ was inserted into the distal rectum by per rectal digital manipulation. The pellet was left as distal as possible to try and avoid imaging interference by the pelvic bones.

### Reproductive System

### Cervix

A porous silicon pellet of 5.04 mm diameter, height 1.94 mm, volume 0.034 cm³, weight 0.04 g and density 0.98 g/cm³ was inserted into the distal cervix via a digital per vaginal method after visualization of the cervical os with a vaginal speculum.

### Left Ovary

The left ovary was identified and located via ultrasonography. 1 ml of pSi/NaCMC suspension was then injected into the left ovary using an ultrasound guided percutaneous technique.

### Urinary System

### Urethra

The urethral opening into the caudal vaginal vault was identified and isolated. A porous silicon pellet of 5.05 mm diameter, height 1.99 mm, volume 0.039 cm³, weight 0.049 and density 1.03 g/cm³ was manually inserted into the urethral orifice, which was subsequently sutured closed to avoid migration of the pellet.

### Left Kidney

The left kidney was identified and located via ultrasonography. 1 ml of pSi/NaCMC suspension was then injected into the left renal pelvis using an ultrasound guided percutaneous technique.

All tissue samples as described above were subjected to CT and MRI imaging. From the CT images generated, the samples were clearly visible in the systems studied, whether viewed through a bone or soft tissue window. The pellet and injections were consistently identified within soft tissue structures when the images were examined on soft tissue windows. However the size and shape of the pellet and injected material were generally best confirmed on bone windows. Some problems were encountered with the images obtained with the respiratory system. Excellent, or at least good, well defined visibility was generally achieved in connection with the lymphatic, vascular, gastrointestinal and genitourinary systems.

From the MRI images generated, the samples were clearly visible in the lymphatic and vascular systems. The imaging sequences were obtained in different planes. T1 weighted in the saggital plane and T2 weighted in the transverse plane. Generally, the pellets were clearer on T2 weighted images. Some injections (head and neck region) produced a signal void on T1 weighted images that aided identification of their location.

### Example 8

For details of the materials used, see Example 7. Metal:pSi pellets were prepared at a composition of 10%:90% by weight, density>0.9 gm/cm³. The metals used in the pellets were iron (Fe:pSi), titanium (Ti:pSi), stainless steel (SS:pSi), carbon (C:pSi) and calcium (Ca:pSi). Pellets were prepared by cold compression of a premixed preparation of pSi and metal particles. The force used was typically in the range 16-20 kN. A number of materials were studied as control experiments. A replica tissue marker, was fabricated from 316 surgical grade stainless steel to resemble the Micromark II™ breast tissue biopsy marker. A modified disposable Kopan spring hook localization needle, (breast biopsy hookwire marker) 21 G, 5 cm long was obtained from Cook Medical (reorder number DKBL-21-5.0-A).

Angiografin is a commercially available (Schering Pty Ltd) contrast agent suitable for use with x-ray imaging and is a 65% aqueous solution of meglumine diatrizoate. An injection of 650 mg/ml is equivalent to 306 mg/ml of organically bound iodine. Omniscan is a commercially available MRI imaging agent comprising 15 ml Gadodiamide 4.305 g/15 ml IV injection (7.5 mmol/15 ml) available from Nycomed Australia Pty. Ltd (Lot number: 10161973).

Three portions of bovine muscle tissue (beef topside roast) of approximately 2 kg weight and approximate dimensions 20 cm length, 10 cm width and 5 cm thickness were used.

### Muscle Tissue 1

Using a scalpel, six incisions were made in the side of the muscle tissue sample, equidistant apart, approximately 2 cm deep and extending approximately one fifth of the width into the tissue (i.e. 2 cm width). The incisions were made so that once the pellet was inserted it would be surrounded by soft tissue. Using the forceps and above incisions, a porous silicon pellet of 5.08 mm diameter, height 2.03 mm, volume 0.034 cm³, weight 0.049 and density 0.95 g/cm³ and one pellet from each of the five different metal/pSi composition pellets were inserted into the tissue sample. To ensure there was no air contained within the incisions they were filled with sterile water and closed by pinching the tissue to ensure the pellets were tightly enclosed by the soft tissue.

### Muscle Tissue 2

Using a scalpel, three incisions were made in one side of the muscle tissue sample, equidistant apart, approximately 2 cm deep and extending approximately one fifth of the width into the tissue (i.e. 2 cm width). The incisions were made so that once the pellet was inserted it would be surrounded by soft tissue. Using the forceps and above incisions, the hook-wire, the stainless steel replica tissue biopsy marker and a porous silicon pellet of 5.05 mm diameter, height 1.43 mm, volume 0.028 cm³, weight 0.026 g and density 0.908 g/cm³ were inserted into the muscle tissue sample.

1 mL samples of each of the 1.5 g/ml pSi/NaCMC suspension, the angiografin and the omniscan were drawn up into syringes and subsequently injected into the other side of the muscle tissue sample, using a vertical approach of the needle and to a depth of approximately 2 cm.

### Muscle Tissue 3

1 mL samples of each of the 1.5 g/ml pSi/NaCMC suspension, the angiografin, the omniscan and straight NaCMC were drawn up into syringes and subsequently injected into the side of the muscle tissue sample, using a vertical approach of the needle and to a depth of approximately 2 cm. Using a scalpel, an incision was made into the side of the muscle tissue sample approximately 2 cm deep and extending approximately one fifth of the width into the tissue (i.e. 2 cm width). The incision was made so that once a pellet was inserted it would be surrounded by soft tissue. Using the forceps and above incision, a porous silicon pellet of 5.08 mm diameter, height 1.79 mm, volume 0.036 cm³, weight 0.039 g and density 1.075 g/cm³ was inserted into the tissue sample.

Muscle tissue samples 1 and 2 were subjected to x-ray, CT and ultrasound, and muscle tissue sample 3 was imaged using MRI.

In muscle tissue sample 1, excellent images were achieved in combination with computed radiography and CT imaging, though the carbon doped pellet results were less impressive. Good visibility was achieved with ultrasound and the MRI imaging gave rise mainly to hypointense/signal voids apart from the carbon doped pellet which gave improved results when compared with those obtained in connection with computed radiography and CT imaging.

Using a low kVp technique, all pellets were classified as having “excellent” visibility, whilst a high kVp technique reduced this to “good”. The carbon-doped pellet was more fragile than the other pellets and therefore more difficult to handle which may be why there were some difficulties in identifying the pellet at ultrasound. At CT, all pellets were identified, with all except carbon-doped achieving an “excellent” rating when images using soft tissue parameters. The carbon pellet exhibited “poor” and “fair” imaging characteristics.

MRI produced the expected signal voids in all but two pellets. The pSi (undoped) control pellet shows good, hypointense, visibility under both T1 and T2 weighting, with similar results for the carbon-doped pellet.

The hypointense/signal void behaviour of pSi imaging impeded visibility under MR imaging. No thoraco-abdmino-pelvic administrations were identified, with only T2 weighted imaging of the submandibular lymph node injection achieving a visibility of “good”. There did not appear to be a significant difference in visibility under either T1 or T2 weighting.

In the muscle tissue sample 2 under x-ray, the pSi pellet was not as clearly defined as either the replica breast marker or hookwire, but was similar to the angiografin injection. The 1.5 g/ml pSi injection was clearly seen as a thick, linear well defined density.

Both injection and pellet pSi formulations were clearly visible under ultrasound, exceeding the visibility of all other samples except gadolinium.

At CT, all samples achieved “excellent” visibility when using a soft tissue imaging algorithm. The pSi injection was also clearly seen using a bone window setting, exceeding the visibility of the angiografin. The pellet only achieved a rating of “fair” for the same parameters.

Under MRI, topside 3 demonstrated hypointense findings for all samples under both T1 and T2 weightings. Interestingly, the gadolinium injection was described as a signal void. The pellet was only poorly visible, being consistent with previous findings.

### Example 9

Example 9 illustrates the in vitro visibility of porous silicon in multiple organ systems in cadaveric tissue (adult male entire greyhound cadaver, non-fixed). More specifically, to show that stain-etched (SE) pSi, in both pellet and particulate form, is capable of providing in-vitro soft tissue visibility in all major body organ systems using the imaging modalities of x-ray and ultrasound.

This was achieved by inserting pelleted and particulate stain-etched pSi solutions into different areas of an entire canine cadaver, and then subjecting the cadaver to imaging under the modalities of radiography and ultrasonography. Results indicated that stain-etched porous silicon, in both pellet and particulate form, can be well visualized in most organ systems under the modalities of x-ray and ultrasound.

All powders were sourced from pSiMedica in Malvern, UK. More specifically, the silicon samples used in this trial were stain-etched, jet milled, non P-doped “cold” poly-silicon powder (“Brachysil™”), 30 μm sized particles, supplied by High Force via pSimedica (Batch number CT7842R9C). Stain-etched (“Brachysil”) pellets were prepared by the cold compression of non-doped “Brachysil” pSi powder (powder batch number CT7842R9C). All pellets were produced using 10% (by weight) cocoa butter to try and reduce pellet fragility. Carboxymethylcellulose sodium salt for use in formulations (medium viscosity) was obtained from Fluka Biochemika, Item no 21902. Ultra Water Soluble transmission ultrasound gel was obtained from Medtel, Lot 0302N4Cl. Water for injection BP 100 ml (pH 5-7) was obtained from Pharmacia & Upjohn.

Ultrasonography was conducted with an Acuson Sequoia 512 ultrasonography unit. Radiography was performed with a Siemans Gigantos-optimatic x-ray system linked to an Agfa computed radiography system and a CR 25.0 digitizer.

All powders (and pellets) were opened and left to stand in a fume-hood for 5-10 minutes. The carboxymethylcellulose sodium salt (NaCMC) was made up into a 0.5% w/v formulation by weighing out 0.1 g of the powder and mixing it with 20 mls of sterile water for injection. 10 ml of the NaCMC solution was added to 15 g of the 30 μm stain-etched poly-Si powder and mixed using a spatula and a vortex mechanical mixer until a homogeneous 1.5 g pSi/ml NaCMC suspension was achieved. In some of the experiments, the 1.5 g pSi/ml NaCMC suspension proved quite difficult to inject. The pSi/NaCMC suspension and the remaining NaCMC were then transferred to glass vials sealed with an air-tight rubber injection membrane and metal seal to allow storage for later use.

### Vascular System

### Right Jugular Vein

The right jugular vein was identified and the overlying skin was shaved devoid of hair. A section of the jugular vein was then isolated by the use of two percutaneously placed suture ligatures, spaced 2 cm apart which occluded the vein. A stab incision was made through the skin and into the lumen of the ligated section of the jugular vein. All blood and blood clots were evacuated via manipulation. The lumen of the vein was then filled with ultrasound gel so as to displace any trapped air and a porous silicon pellet of 5 mm diameter, height 1.38 mm, volume 0.027 cm³, weight 0.048 g and density 1.772 g/cm³ was then inserted through this incision. The incision was sutured closed.

### Left Jugular Vein

The left jugular vein was identified and the overlying skin was shaved devoid of hair. A section of the jugular vein was then isolated by the use of two percutaneously placed suture ligatures, spaced 2 cm apart which occluded the vein. A 22 g 1-inch IV catheter was then inserted into the lumen of the ligated section of vein, and all blood and blood clots were evacuated. 1 ml of the pSi/NaCMC suspension was injected into the area described above, and the catheter was subsequently removed.

### Respiratory System

### Right Nostril

The right nostril was filled with ultrasound gel before a porous silicon pellet of 5 mm diameter, height 1.45 mm, volume 0.028 cm³, weight 0.049 g and density 1.721 g/cm³ was inserted into the rostral most section of the right nasal cavity. The right nostril was then packed off with gauze swabs

### Left Nostril

The left nostril was filled with ultrasound gel before 1 ml of the pSi/NaCMC suspension was inserted into the rostral most section of the left nasal cavity. The left nostril was then packed off with gauze swabs.

### Lymphatic System

### Right Submandibular Lymph Node (SMLN)

The right SMLN was identified and the overlying skin was shaved devoid of hair. A stab incision was made through the skin and into the SMLN. A porous silicon pellet of 5 mm diameter, height 1.35 mm, volume 0.026 cm³, weight 0.047 g and density 1.773 g/cm³ was inserted into the middle of the lymphoid tissue, and the incision was filled with ultrasound gel to displace any trapped air. The incision was sutured closed.

### Left Submandibular Lymph Node

The left SMLN was identified and the overlying skin was shaved devoid of hair. 1 ml of pSi/NaCMC suspension was then percutaneously injected into the LN through a 16 g needle and 3 ml syringe.

### Alimentary System

### Oesophagous

Ultrasound gel was used to fill the cranial oesophagous via a digital per os method after visualization of the oesophageal opening with a laryngoscope. A porous silicon pellet of 5 mm diameter, height 1.36 mm, volume 0.027 cm³, weight 0.047 g and density 1.76 g/cm³ was then inserted into the cranial oesophagous before it was packed off with gauze swabs.

### Rectum

2 mls of the pSi/NaCMC suspension was inserted into the caudal rectum & anus. The injection was performed as distal as possible to try and avoid imaging interference by the pelvic bones. The anus was then packed off with gauze swabs.

### Small Intestines

An ultrasound guided percutaneous injection of pSi/NaCMC suspension was attempted into the lumen of a section of small intestine, but was unsuccessful due to the inability of the needle to puncture the intestinal wall.

### Stomach

An ultrasound guided percutaneous injection of 1 ml pSi/NaCMC suspension was attempted into the gastric lumen. The injection was successful, but was not able to be imaged by ultrasound due to interference created by gas accumulation within the stomach.

### Reproductive System

### Right Testicle

A stab incision was made through the skin and into the body of the right testicle. A porous silicon pellet of 5 mm diameter, height 1.31 mm, volume 0.026 cm³, weight 0.045 g and density 1.75 g/cm³ was inserted into the incision, which was subsequently filled with ultrasound gel to displace any trapped air. The incision was then sutured closed routinely.

### Left Testicle

1 ml of pSi/NaCMC suspension was percutaneously injected into the left testicle through a 16 g needle and 3 ml syringe.

### Urinary System

### Urethra

A porous silicon pellet of 5 mm diameter, height 1.44 mm, volume 0.028 cm³, weight 0.049 g and density 1.733 g/cm³ was manually inserted into the distal urethra of the penis, which was subsequently filled with ultrasound gel and sutured closed to avoid migration of the pellet.

### Left Kidney

The left kidney was identified and located via ultrasonography. 1 ml of pSi/NaCMC suspension was then injected into the left renal pelvis using an ultrasound guided percutaneous technique.

Ultrasound imaging was carried out using a linear array 15 MHz through 8 MHz transducer with imaging set at close focus. The radiography examinations were carried out with fine focus, grid, and focus-to-film distance of 100 cm. Exposures of between 56 and 85 kVp and 12 to 40 mAs were used depending on the body part under examination. The Computerized Radiography system plate speed was set to detail with exposure classifications 200 and 300 according to the body part under examination.

In summary, SE porous silicon in both pellet and particulate form has been shown in in-vivo tissue trials to be radiographically obvious on X-ray and clearly echogenic on ultrasound in all body systems. The pSi samples have demonstrated a dense echogenic reflection on ultrasound and visible density on x-ray. This means that stain etched pSi in both pellet and particulate form was conspicuous on all tested imaging modalities. The level of visibility across the x-ray modality was shown to be greatly improved by the use of the less porous, and therefore denser, stain-etched pSi material. Ultrasonography appears to be a particularly promising imaging modality for visualizing pSi pellets and particulate suspensions. Problems associated with air entrapment around samples can be reduced by refinement of the surgical implantation technique and are likely to be less of an issue in-vivo due to the natural healing process of living tissue reducing any artefact that air may cause. It was also noted that re-suspension with stock solution or making up with small amounts of straight NaCMC immediately prior to use were beneficial in connection with formulations comprising NaCMC.

### Example 10

Example 10 illustrates the use of porous silicon to mark the skin and which can be visualized in the form of a tattoo. This was achieved by introducing small quantities of particulate pSi into the dermis layer of a tissue sample. A particulate pSi suspension was introduced into the dermis layer of a skin tissue sample to create a tattoo that was easily visible to the naked eye.

All powders were sourced from pSiMedica in Malvern, UK. More specifically, the silicon samples used in this trial were stain-etched, jet milled, non P-doped “cold” poly-silicon powder (“Brachysil™”), 30 μm sized particles, supplied by High Force via pSimedica (Batch number CT7842R9C).

Carboxymethylcellulose sodium salt for use in formulations (medium viscosity) was obtained from Fluka Biochemika, Item no 21902. Water for injection BP 100 ml (pH 5-7) was obtained from Pharmacia & Upjohn. Digital image capture was performed with a Fuji Finepix S602, 3.1 mega pixel digital camera. A 1.5 kg porcine muscle tissue sample with the skin present and intact was used in this experiment.

All powders (and pellets) were opened and left to stand in a fume-hood for 5-10 minutes. The carboxymethylcellulose sodium salt (NaCMC) was made up into a 0.5% w/v formulation by weighing out 0.1 g of the powder and mixing it with 20 mls of sterile water for injection. 10 ml of the NaCMC solution was added to 15 g of the 30 μm stain-etched poly-Si powder and mixed using a spatula and a vortex mechanical mixer until a homogeneous 1.5 g pSi/ml NaCMC suspension was achieved. The pSi/NaCMC suspension and the remaining 10 ml of NaCMC were then transferred to glass vials sealed with an air-tight rubber injection membrane and metal seal to allow storage for later use.

The tattooing procedure was performed using a non-electrical, hand held tattooing punch with needles of steel that insert the ink into the skin dermis. This device is routinely used to tattoo the symbol “φ” into the ears of cats and dogs to indicate that they have been neutered.

The skin on the porcine muscle tissue was cleaned to remove debris, and was undermined from the underlying muscle to allow the block section of the tattoo punch to be inserted under the skin. A tattoo was placed into the skin using standard tattooing ink to act as a comparison control for the pSi skin marking. This was done by covering the target area with tattoo ink and then inserting the ink into the dermis with the tattoo punch. The resulting wound was then rubbed with ink to ensure pigment take-up and then cleaned to remove excess ink. A tattoo was then placed into the skin of the tissue sample using the 1.5 g/ml pSi/NaCMC suspension as the tattoo ink/pigment. This was done by covering the target area with the pSi suspension and then inserting it into the dermis with the tattoo punch. The resulting wound was then rubbed with the pSi suspension to ensure pigment take-up and then cleaned to remove excess suspension.

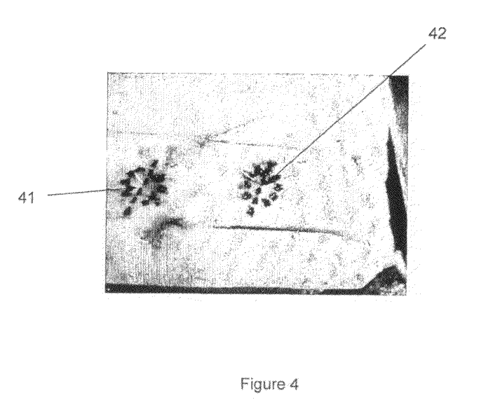

The control and porous silicon tattoos are shown in FIG. 4. The control tattoo (41) is pictured on the left of the image and the porous silicon tattoo (42) on the right. It was observed during the trial that the suspension of 1.5 g/ml of pSi in NaCMC was very similar in consistency and characteristics to the normal tattoo ink. Advantageously, the pSi tattoo may be loaded with antibiotic to minimize the risks of infection. Loading may utilize the techniques described in WO 05042023 the contents of which are hereby incorporated by reference in their entirety.

The in-vitro tissue trials show that porous silicon in particulate suspension can be used to mark the skin in the form of a tattoo. It has also been shown that pSi as a skin marking agent provides substantially equivalent visibility when compared to normal tattoo ink.

### Example 11

This example describes the use of porous silicon as an ultrasound contrast agent, and compares it to a commercially available contrast agent. The results obtained indicate that porous silicon particles used in this way can generate echo enhancement at least as good as, if not greater than, presently available ultrasound contrast agents.

Stain-etched, jet milled, p-doped “cold” poly-silicon powder, possessing a d₅₀ of 30 μm with average porosity of 5 vol % was used at a w/v concentration of 0.05%. The porous silicon particles were suspended in a 0.5% solution of carboymethylcellulose, sodium salt (NaCMC). Commercial ultrasound contrast agent (Levovist™) at a 400 mg/ml formulation as per manufacturer's instructions was used for comparison. 0.5% NaCMC was used as a negative control. 1 ml aliquots of each material were injected into muscle tissue samples using an 18 g needle. Imaging was undertaken using a Terason 2000 ultrasound machine, equipped with a 10 L5 128 element linear ultrasound transducer. Imaging was undertaken at 10 MHz, with focus set to 1.3-2 cm. Water soluble ultrasound transmission gel, was used to acoustically couple the transducer to the tissue samples. During image acquisition the ultrasound probe was held stationary with respect to the tissue samples and the needle by clamping it in a retort stand. Images were captured immediately prior to the injection (“pre-injection” image), and immediately following the injection (“post-injection image”). All imaging parameters were held constant during acquisition.

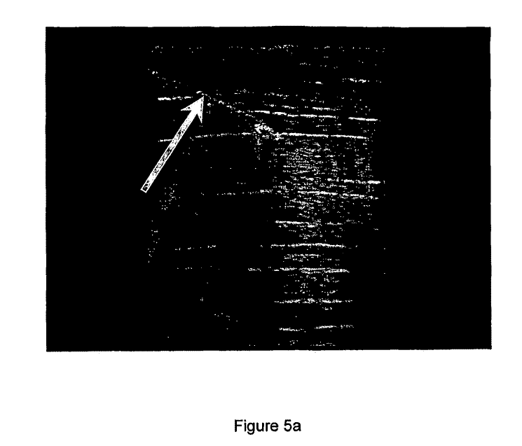

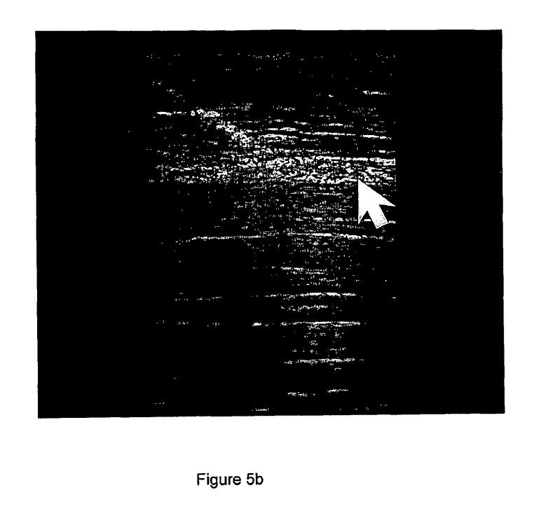

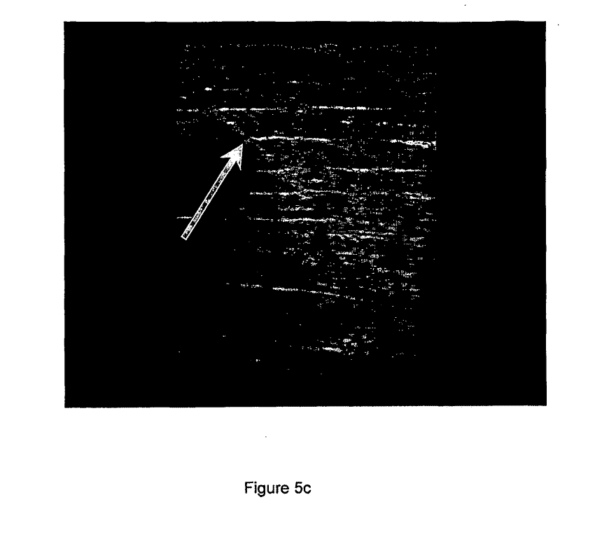

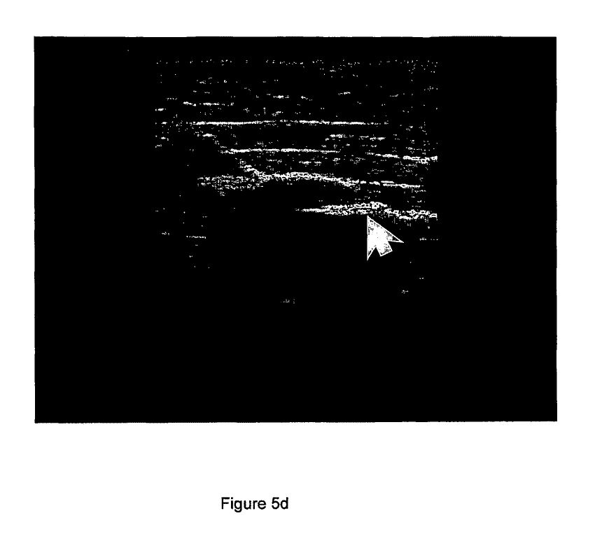

The images generated are shown in FIGS. 5a-d. In all images, the needle is seen as a linear echogenic shadow to the left (arrow) and the area of enhancement to the right (arrowhead). Low concentrations of pSi formulated in 0.5% NaCMC demonstrate strong echogenic enhancement which is equal to or exceeds that of a presently available contrast agent. FIGS. 5a and b illustrate the porous silicon sample pre and post-injection respectively and FIGS. 5c and 5d illustrate the commercial sample pre and post injection.

### Example 12

This example describes the labelling of porous silicon particles with antibody proteins and the subsequent imaging of these labelled particles in association with particular cells. The results obtained indicated that porous silicon particles can be labelled with antibody and used to recognize and bind to a target cell and assist in selectively imaging that cell, thus providing the basis for marking and imaging target tissues at the cellular level.

Anodised pSi powder, jet-milled and classified to d₅₀ 8.1 μm (d₁₀ 1.6 μm, d₉₀ 20.2 μm) with average porosity 70 vol % was used. The hydrophillicity of the particle surfaces had been increased by hydrosilylation. Porous silicon microparticles were dried by high vacuum, purged and stored under high quality argon. 1-butenoic acid and mesitylene were redistilled onto molecular sieves under argon and a 30-50% (by volume) solution made up. Oxygen was removed by freeze/pump/thaw (repeated four times) and the solution stored under argon until use. To 1 g of pSi in a schlenck flask, 5 ml of 30-50% 1-butenoic acid solution was added and the mixture brought to 100° C. The reaction was stirred (slowly) under argon for 96 hours with periodic aliquots removed for Fourier transform infrared spectroscopy and dispersability assessment. At 96 hours, the reaction was brought to room temperature and the derivatized particles allowed to settle. The solution was removed by pipette and the pSi washed twice with dichloromethane (suspension/centrifugation/decant) and twice with ethyl acetate followed by drying at room temperature under a stream of argon. After final FTIR analysis the derivatized material was stored in a dessicator until use.

Three muscle-specific surface markers were selected due to their upregulated expression in differentiated muscle cells (C2C12), and their lack of expression in a non-muscle cell-line (3T3 fibroblasts). The latter acted as a negative control. The muscle-specific surface markers used were surface marker primary antibodies, i.e. Integrin α-7, M-Cadherin or Pan Laminin. Integrin α-7 and M-Cadherin were obtained from Santa Cruz and Pan Laminin was obtained from Sigma.

The secondary antibodies used in connection with Integrin alpha-7 and M-cadherin were Donkey anti-Goat ALEXA 488 and the secondary antibodies used in connection with Laminin were Goat anti-Rabbit ALEXA 488.

The binding of the primary antibodies to the pSi particles was achieved by incubation in an aqueous environment for 12-36 hours and was confirmed using fluorescently-linked antibodies. Both of the secondary antibodies were obtained from Invtrogen Molecular Probes, Mount Waverley, Victoria, Australia.

The C2C12 mouse myoblast cell line was obtained from ATCC, see www.atcc.org. Cells were grown on collagen-coated glass chamber slides in growth media consisting of Dulbecco's modified Eagle's medium (DMEM), 10% fetal bovine serum (FBS), 4 mM L-glutamine, 100 IU/ml penicillin, and 100 μg/ml streptomycin. The fusion of myoblasts into multi-nucleated myotubes was induced by changing the media composition to DMEM, 2% horse serum, 5 μg/ml linoleic acid, 50 ng/ml IGF-1, 4 mM L-glutamine, 100 IU/ml penicillin, and 100 μg/ml streptomycin.

A 3T3 mouse fibroblast cell line was used as a negative control for the muscle markers used in this trial. Cells were cultured under the same growth conditions as for the C2C12 cells discussed above. The cells were fixed with 3% formaldehyde in phosphate buffered saline (PBS) for 15 mins at room temperature. Chamber slides were then sealed in parafilm to prevent dehydration and stored at 4° C. until required for particle labeling experiments.

The binding of marked particles to cells was visualized using light (phase contrast) and fluorescent microscopy (Nikon Diaphot inverted fluorescent microscope with associated software).

Porous Si particles were suspended in PBS at 5% w/v and labelled with either Integrin α-7 (A), M-cadherin (B) or Laminin (C) primary antibodies at a pSi: antibody concentration of 1:10 for 12-36 hours at 4° C. The incubated particles were then washed twice with PBS by centrifugation at 13000 rpm for 2 mins to pellet the particles and then resuspended in PBS. The labelled particles were then washed, diluted to 0.5% w/v and incubated with fused C2C12 cells at a 0.1% w/v concentration for 2 hours. Following a 1-hour incubation with secondary antibody, bound particles were visualised using the inverted fluorescent microscope.

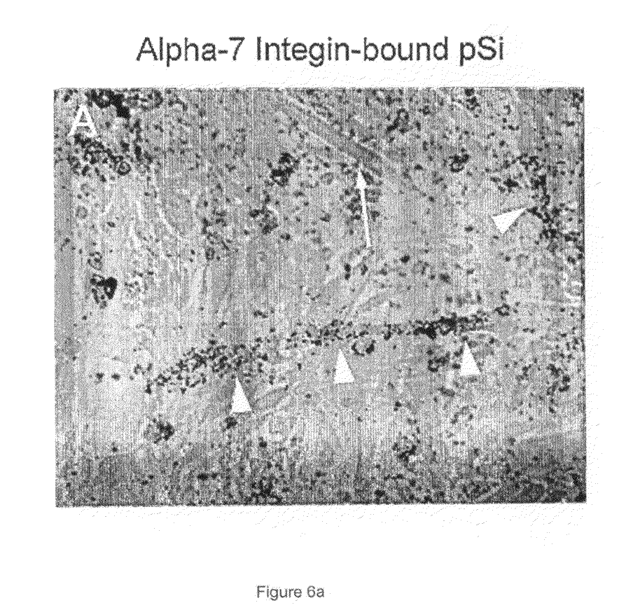

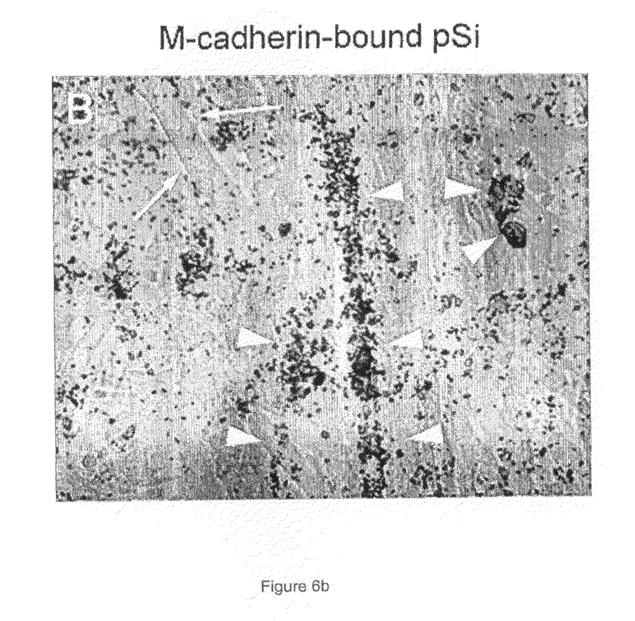

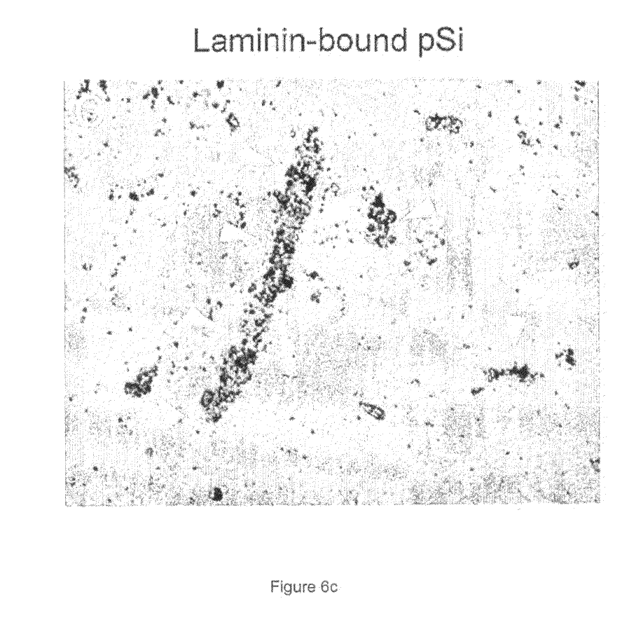

The images generated are shown in FIGS. 6a-c. Myotubes coated with labelled particles were observed for all antibodies (indicated by arrowheads), although the binding and number of labelled myotubes was higher for M-cadherin (B) and Laminin (C) than Integrin α-7 (A). The full arrows indicate unlabelled myotubes. Distinct areas of binding to small myotubes were still observed.

Labelled particles were then washed, diluted to 0.5% w/v and incubated with 3T3 fibroblasts in parallel to fused C2C12 cells for 2 hours. Following a 1-hour incubation with secondary antibody, bound particles were visualised using the inverted fluorescent microscope. No cell-particle binding was observed for any of the antibodies.

### Example 13

Example 13 illustrates the imageability of silicon using nuclear medicine imaging techniques. Technetium-99 m and iodine-131 were incorporated into porous silicon, and imaged using conventional gamma cameras. In order to incorporate I-131, I-131 radiolabelled antibodies were used.

All powders were sourced from pSiMedica in Malvern, UK. More specifically, the silicon sample used in this example was classified anodized silicon possessing a d₅₀ of 58.2 μm, 70 vol % porosity (batch number CT8168R5C). Other materials used in this example were: 200 MBq Na pertechnetate (Tc-99 m) solution; 200 MBq NaI (I-131) solution; 20 μl Pan-laminin antibody (Sigma, catalogue no. L9393).

The gamma camera used was a Philips ADAC SOLUS and the SPECT/CT scanner was a GE Hawkeye Infinia. Image orientation was confirmed using two marker sources: Co-57 marker (Tc-99 m samples) and Ba133 marker (I-131 samples).

Other materials used in this example were two bovine muscle tissue samples (beef topside roasts) which were approximately 2 kg weight and of approximate dimensions 20 cm length, 10 cm width and 5 cm thickness. The water used for making up formulations suitable for injection was BP (100 ml, pH 5-7). The antibody used was a Pan-laminin antibody which was commercially available from Sigma (cat L9393).

The formulation comprising Technetium-99 m was made up as follows. All powders (and pellets) were opened and left to stand in the fume-hood for 5-10 minutes. Eppendorf tubes were labelled with unique identifiers and 10 mg of pSi was added to two of the tubes followed by 200 MBq of Na techperchnetate solution (150 μl). The tubes were incubated at room temperature and the solution transferred into a 1 ml syringe. The solution was filtered and flushed with air. The activity was measured on the filtrate (sample a), the filter and the syringe. The filter was rinsed with 200 μL of water and flushed with air. The activity was measured on the filtrate (sample b) and the filter. If activity was found on the filter, the filter was reverse flushed with 1 ml of water (filtrate sample c). The activity collected on the filtrate (sample c) and the filter was measured.

The formulation comprising Iodine 131 was made up as follows. 10 mg of the anodised pSi was placed in an Eppendorf tube. 200 MBq of I-131 solution (150 μl) was added at the required pH and the vial was incubated at room temperature. The solution was transferred into a 1 ml syringe and the solution filtered and flushed with air. The activity was measured on the filtrate (sample b) and the filter. If activity was found on the filter, the filter was reverse flushed with 1 ml of water (filtrate sample c). The activity collected on the filtrate (sample c) and the filter was measured.

Labelling of the anti-laminin Ab with iodine-131 was performed using the Chloramine-T method. For details of this method, see Hunter W. M. and Greenwwod “F. C. Preparation of iodine-131 labelled growth hormone of high specific activity” in Nature 194: 495-6, 1962. 5 μL of the Ab solution (0.5 mg/mL) was diluted in 45 μL of phosphate buffer 0.1M pH7 in an Eppendorf tube to which was added 10-15 μL of the Na-1131 solution and 12.5 μL of Chloramine-T solution (1 mg/mL in phosphate buffer 0.1M pH7). The solution was stirred at room temperature for 3 min. 25 μl of a sodium metabisulfite solution (15 mg/ml in water) was added to stop the reaction. For the purposes of quality control, after 5 min an instant thin-layer chromatography-silica gel was performed (in 85% MeOH/25% H₂O). After dilution in 1.5 ml of phosphate buffered saline (PBS) 0.15M pH 7.2, the reaction mixture was applied on a gel filtration column (Sephadex G-25 PD-10). The column was eluted in aliquots of 1 ml. The purified I-131 was eluted in fractions 2, 3 and 4, fraction 3 being the most concentrated fraction used for the labelling of the pSi.

The activity and stability trials indicated the silicon samples retained significant levels of activity, up to about 80%.

Labelled silicon samples comprising 43.3 MBq Tc-99 m and 28.9 MBq I-131 were injected in two bovine muscle tissue samples (topside roast samples). Reference samples (un-bound 48.9 MBq Tc-99 m pertechnetate and 27.2 MBq I-131 sodium iodide solutions) were also injected to provide comparative imaging of the labelled porous silicon samples.

Imaging was performed using the SPECT/CT and gamma cameras. SPECT CT imaging took approximately 42 minutes, and 5 minute static acquisitions were performed in the gamma camera. CT imaging was performed at 140 kVp, 2.5 mA, helical acquisition with 10 mm reformatting. Tc-99 m imaging was performed using the low energy high resolution gamma camera collimators, while the I-131 used high energy general purpose collimators. Clear images were obtained using both SPECT/CT and planar techniques illustrating that porous silicon provides imageability equivalent to that of existing radiotracers. More specifically, anodized porous silicon in particulate form can be associated or bound to gamma emitting isotopes and can be successfully imaged using conventional imaging techniques.

### Example 14

Example 14 illustrates the imageability of silicon using nuclear medicine imaging techniques. Fluorine-18 was incorporated into porous silicon, and imaged using a PET camera.

All powders were sourced from pSiMedica in Malvern, UK. The materials used in this trial were classified anodized silicon which possessed a d₅₀ of 58.2 μm and 70 vol % porosity (batch number CT8168R5C), a 95.3 MBq F-18 solution and a PET scanner (Philips Allegro). Other materials included a bovine muscle tissue sample (beef topside roast) of approximately 2 kg and of approximately 20 cm length, 10 cm width and 5 cm thickness. Water suitable for making formulations suitable for injection was obtained from Pharmacia & Upjohn, BP 100 ml, pH 5-7. All operations using radioactive material were to cGLP standards.

All powders (and pellets) were opened and left to stand in the fume-hood for 5-10 minutes. A number of Eppendorf tubes were uniquely labelled. 10 mg of pSi was added to a number of the tubes. 95.3 MBq of fluorine-18 solution (150 μL) was added to each tube and the vials were incubated at room temperature. The solution was transferred to a 1 ml syringe and the solution filtered and flushed with air. The activity was measured on the filtrate sample the filter and the syringe. The filter was rinsed with 200 μl of water and flushed with air. The activity was measured on filtrate samples and the filter. If activity was found on the filter, the filter was reverse flushed with 1 ml of water. The activity was measured collected on the filtrate sample and the filter. The measurements indicated that the majority of the F-18 is retained on the pSi.

The porous silicon labelled with F-18 and a reference sample of unbound NaF-18 solution were injected into bovine muscle tissue samples (beef topside roast samples). Imaging was performed using the PET scanner. An emission/transmission acquisition lasting 15 minutes was subsequently reformatted using normal clinical protocols. The porous silicon labelled with F-18 produced clear PET images which were equivalent to that of existing PET radiotracers.

1. A method of imaging a human or animal subject, wherein the contrast of the image is enhanced by administering an imaging agent comprising silicon to the human or animal subject.

2. A method according to claim 1, wherein the imaging agent consists of or consists essentially of silicon.

3. A method according to claim 1, wherein the silicon is about 98 to 99.999999% pure.

4. A method according to claim 1, wherein the silicon comprises one or more of amorphous silicon, single crystal silicon, polycrystalline silicon and bulk crystalline silicon.

5. A method according to claim 1, wherein the silicon is porous silicon.

6. A method according to claim 5, wherein the porous silicon is selected from one or more of stain etched porous silicon, gas etched porous silicon or anodised porous silicon.

7. A method according to claim 6, wherein the porous silicon is stain etched porous silicon.

8. A method according to claim 5, wherein the porous silicon is selected from one or more of microporous silicon, mesoporous silicon or macroporous silicon.

9. A method according to claim 8, wherein the silicon is biodegradable or resorbable.

10. A method according to claim 1, wherein the silicon is photoluminescent in the visible and/or near infrared.

11. A method according to claim 5, wherein the porous silicon comprises or consists essentially of surface modified silicon.

12. A method according to claim 11, wherein the surface modified porous silicon comprises or consists essentially of one or more of: derivatised porous silicon, partially oxidised porous silicon, porous silicon with silicon hydride surfaces.

13. A method according to claim 5, wherein the porous silicon comprises or consists essentially of unmodified silicon.

14. A method according to claim 1, wherein the silicon comprises, micro or nano-particulate silicon.

15. A method according to claim 14, wherein the silicon comprises nanoparticles which are agglomerated or consolidated.

16. A method according to claim 1, wherein the imaging agent comprises one or more further components.

17. A method according to claim 16, wherein the one or more further components include one or more metals and/or optionally, isotopes thereof.

18. A method according to claim 17, wherein the one or more metals and/or optionally isotopes thereof, are selected from cadmium, cesium, cobalt, copper, gallium, lead, manganese, molybdenum, niobium, indium, zirconium, yttrium, lutetium, rubidium, ruthenium, scandium, technetium, titanium, gold, tantalum, iridium, platinum, tungsten, rhodium, palladium, silver, iron, gadolinium, chromium, zinc, barium, magnesium, calcium, strontium, samarium, thallium, holmium, scandium.

19. A method according to claim 16, wherein the one or more further components include stainless steel.

20. A method according to claim 16, wherein the one or more further components include one or more non-metals, and/or optionally, isotopes thereof, selected from bromine, carbon, fluorine, hydrogen, iodine, nitrogen, oxygen, selenium, phosphorus, xenon, chlorine.

21. A method according to claim 20, wherein the one or more further components is phosphorous.

22. A method according to claim 21, wherein the phosphorous is ³¹P.

23. A method according to claim 16, wherein the one or more further components include one or more gases and/or optionally, isotopes thereof.

24. A method according to claim 23, wherein the one or more gases, and/or optionally, isotopes thereof are selected from: nitrogen; oxygen; carbon dioxide; hydrogen; nitrous oxide; a noble or inert gas, for example, helium, neon, argon, radon, xenon or krypton; a radioactive gas; a hyperpolarized noble gas, for example, hyperpolarized argon; a low molecular weight hydrocarbon; a cycloalkane; an alkene; an alkyne; an ether; a ketone; an ester; sulfur-based gases; halogenated gases, for example, partially fluorinated gases or completely fluorinated gases; air and air/perfluorocarbon mixtures.

25. A method according to claim 16, wherein the one or more further components include one or more radionuclides.

26. A method according to claim 1, wherein the imaging agent is combined with a pharmaceutically acceptable carrier, excipient or diluent.

27. A method according to claim 26 wherein the imaging agent is combined with one or more of a solubilizing agent, wetting agent, solvent, surfactant, detergent, phospholipid, dissolution enhancing excipient, emulsifying agent, emulsion stabilizer, stabilizing agent, suspending agent, humectant, gelling agent, stiffening agent, thickening agent, viscosity increasing agent, binder, lubricant, alkalizing agent, glidant, adhesive, coating, film forming agent, encapsulant, plasticizer, flavouring agent, flavour enhancer, taste masking agent, sweetening agent, acidulant, colour, opacifying agent, preservative, acidifying agent, adsorbent, alcohol denaturant, antiadherent agent, anticaking agent, antifoaming agent, antioxidant, buffering agent, chelating agent, dispersing agent, emollient, esterifying agent, penetration enhancer, sequestering agent, water absorbing agent, water repelling agent.

28. A method according to claim 26, wherein the silicon is present in an amount, or equivalent amount, of 0.001 g per ml of total formulation, up to about 2.2 g/ml.

29. A method according to claim 28, wherein the silicon is present in an amount, or equivalent amount, of 0.005 g per ml of total formulation, up to about 1.5 g/ml.

30. A method according to claim 29, wherein the silicon is present in an amount, or equivalent amount, of 0.05 g per ml of total formulation, up to about 0.5 g/ml.

31. A method according to claim 30, wherein the porosity of the porous silicon is about 70 vol %.

32. A method according to claim 1, wherein the imaging agent is in a form suitable for use in one or more of the human or animal vasculature system, the respiratory system, the lymphatic system, the alimentary system, the nervous system, the reproductive system, the renal/urinary system and wherein one or more of said systems is imaged.

33. A method according to claim 32, wherein the imaging agent comprises silicon particles which are all or substantially all about 0.1 nm to about 1000 μm in diameter.

34. A method according to claim 33, wherein the diameter is about 0.5 nm to 300 μm.

35. A method according to claim 32, wherein the imaging agent is in a form suitable for use in the vasculature and the imaging agent comprises silicon particles which are all or substantially all no larger than about 10 μm in diameter.

36. A method according to claim 35, wherein the silicon particles are all or substantially all about 5 μm or less in diameter.

37. A method according to claim 36, wherein the diameter is 500 nm or less.

38. A method according to claim 1, wherein the imaging agent comprises or consists essentially of Brachysil™.

39. A method according to claim 1, wherein the imaging agent is in a form suitable for use as a tissue marker and said tissue marker is delivered to an anatomical site.

40. A method according to claim 39, wherein the tissue marker is in particulate or pellet form.

41. A method according to claim 40, wherein the tissue marker is in particulate form and the silicon particles possess an average size in the range from about 10 nm to 200 μm.

42. A method according to claim 41, wherein the average size is in the range from about 5 μm to about 100 μm.

43. A method according to claim 40, wherein the tissue marker is in the form of a pellet and has a major dimension of about 0.1 mm to 5 cm.

44. A method according to claim 40, wherein all of the dimensions of the pellet are less than about 0.5 cm.

45. A method according to claim 40, wherein the tissue marker is in pellet form and the shape of the pellet is selected from spheres, irregular shapes, discs, cylinders, rods, strips, barbs, lozenges, ovals.

46. A method according to claim 39, wherein the tissue marker is used to mark one or more of the following tissues: colon, rectum, prostate, breast, brain, kidneys, liver, lungs, bone, oropharynx, skin, lymph nodes, adrenals, testis, ovaries, ureter, nerve, bladder, heart, spleen and soft tissues in general including muscles.

47. A method according to claim 39, wherein the tissue marker is implanted under the guidance of one or more of ultrasonic imaging, fluoroscopy, optical imaging, fluorescence imaging, thermal imaging, CT, MRI, x-ray.

48. A method according to claim 47, wherein the tissue marker is implanted under the guidance of ultrasonic imaging.

49. A method according to claim 39, wherein the tissue marker is used to mark the site of a biopsy.

50. A method according to claim 39, wherein the tissue marker is used to monitor physiological changes within a tissue or the site of a biopsy.

51. A method according to claim 39, wherein the density of the tissue marker is greater than about 0.5 g/cm³

52. A method according to claim 51, wherein the density of the tissue marker is greater than about 0.8 g/cm³.

53. A method according to claim 51, wherein the porosity of the porous silicon is about 50 vol %.

54. A method according to claim 1, wherein the imaging agent is used as a positioning aid.

55. A method according to claim 46, wherein the tissue marker is used to mark the skin and is in the form of a tattoo.

56. A method according to claim 55, wherein the tattoo is loaded with antibiotic.

57. A method according to claim 1, wherein the imaging agent is in a form suitable for use in molecular imaging.

58. A method according to claim 57, wherein the imaging agent is combined with an imaging probe.

59. A method according to claim 1, wherein the method comprises the use of one or more of a range of modalities selected from one or more of x-ray, CT, gamma scintigraphy, PET scintigraphy, optical imaging, fluorescence imaging, thermal imaging, infrared, ultrasound, MRI.

60. A method according to claim 59, wherein the method comprises the use of one of the following combinations of modalities: CT and ultrasound; x-ray and ultrasound; CT and MRI; MRI and ultrasound; PET scintigraphy and CT; gamma scintigraphy and CT; PET scintigraphy and MRI; gamma scintigraphy and MRI.

61. A method according to claim 1, wherein the method comprises the use of ultrasound.

62. A method according to claim 1, wherein the method comprises the use of CT.

63. A method according to claim 1, wherein the method comprises the use of x-ray.

64. A method according to claim 1, wherein the method comprises the use of MRI.

65. A method according to claim 1, wherein the method comprises the use of thermal imaging.

66. A method according to claim 1, wherein the method comprises the use of gamma scintigraphy.

67. A method according to claim 1, wherein the method comprises the use of PET scintigraphy.

68. A method according to claim 1, wherein the method comprises the use of optical imaging.

69. A method according to claim 1, wherein the method comprises the use of fluorescence imaging.

70. A method according to claim 1, wherein the method comprises the use of infrared.

71. A method according to claim 61, wherein the imaging agent comprises microbubbles or microspheres of silicon of less than or equal to about 20 μm in diameter.

72. A method according to claim 1 which further comprises the diagnosis and/or monitoring, and/or treatment of a disease, condition or injury.

73. A method according to claim 72, wherein the treatment is monitored.

74. A method according to claim 40, wherein the imaging agent comprises or consists essentially of Brachysil™.

75. A method according to claim 1, wherein the contrast of the image is enhanced through the use of positive contrast.

76. A method according to claim 1, wherein the contrast is enhanced through the use of negative contrast.

77. A method of imaging a human or animal subject, wherein the contrast of the image is enhanced by administering a biodegradable imaging agent comprising, or consisting essentially of, or consisting of porous silicon, and which is imageable or imaged with more than one modality, to the human or animal subject.

78. A method according to claim 77, wherein the biodegradable imaging agent is a tissue marker.

79. A method according to claim 77, wherein the biodegradable imaging agent is a molecular imaging agent.

80. A method according to claim 77, wherein the biodegradable imaging agent is a contrast agent suitable for use in one or more of the human or animal vasculature system, the respiratory system, the lymphatic system, the alimentary system, the nervous system, the reproductive system, the renal/urinary system.

81. A method according to claim 77, wherein complete biodegradation of the porous silicon imaging agent occurs within 29 days following administration.

82. A method according to claim 77, wherein the imaging agent is imageable or imaged with more than two modalities.

83. A method according to claim 82, wherein the imaging agent is imageable or imaged with more than three modalities.

84. A method according to claim 77, wherein the modalities are selected from x-ray, CT, gamma scintigraphy, PET scintigraphy, optical imaging, fluorescence imaging, thermal imaging, infrared, ultrasound, MRI.

85. A method according to claim 84, wherein the modalities are selected from x-ray, CT, ultrasound and MRI.
